(19) United States
(12) Patent Application Publication
(10) Pub. No.: US 2007/0077583 A1
(43) Pub. Date: Apr. 5, 2007

(54) Alpha enolase-directed diagnostics and therapeutics for cancer and chemotherapeutic drug resistance

(75) Inventors: Elias Georges, Laval (CA); Panagiotis Prinos, Montreal (CA)

Correspondence Address: WILMER CUTLER PICKERING HALE AND DORR LLP, 60 STATE STREET, BOSTON, MA, 02109, US

(73) Assignee: Aurelium BioPharma, Inc., Montreal (CA)

(21) Appl. No.: 11/524,780

(22) Filed: Sep. 21, 2006

Related U.S. Application Data
(60) Provisional application No. 60/719,060, filed on Sep. 21, 2005.

(51) Int. Cl. C12Q 1/68; G01N 33/574; A61K 38/16; A61K 38/14; A61K 31/7048; A61K 31/704; A61K 31/7076; A61K 31/7072

(52) U.S. Cl. 435/6; 435/7.23; 424/155.1; 424/450; 514/12; 514/8; 514/34; 514/45; 514/49; 514/492; 514/449; 514/27; 514/575; 424/94.64; 514/263.31; 514/251

(57) ABSTRACT

Disclosed are methods for detecting a neoplasm and/or chemotherapeutic drug resistance or angiogenic potential in neoplastic cells by detecting an increase in the expression of α-enolase in such cells, or in the case of metastatic potential on the surface of such cells, as compared to the level of expression of α-enolase protein in a normal or non-MDR neoplastic cell or on the surface of a non-metastatic neoplastic cell. In addition, methods and a composition are disclosed for increasing the sensitivity of a neoplasm to a chemotherapeutic drug treatment regime, for inhibiting angiogenesis and metastatic potential in chemotherapeutic drug resistant or neoplastic cells, and for inducing apoptosis in chemotherapeutic drug resistant or neoplastic cells.

### FIELD OF THE INVENTION

This invention relates to the field of cancer. In particular, this invention relates to the detection, diagnosis, and treatment of neoplastic cells, and to the detection and treatment of chemotherapeutic drug-resistant neoplastic cells and neoplastic cells that show metastatic potential. Furthermore, this invention relates to increasing apoptosis, inhibiting angiogenesis and metastatic potential in neoplastic and chemotherapeutic drug resistant cells.

### BACKGROUND OF THE INVENTION

Cancer is one of the deadliest illnesses in the United States, accounting for nearly 600,000 deaths annually. This “disease” is in fact a diverse group of diseases, which can originate in almost any tissue of the body. In addition, cancers may be generated by multiple mechanisms including pathogenic infections, mutations, and environmental insults (see, e.g., Pratt et al. (2005) Hum. Pathol. 36(8): 861-70). The variety of cancer types and mechanisms of tumorigenesis add to the difficulty associated with treating a tumor, increasing the risk posed by the cancer to the patient's life and wellbeing.

Diseases such as cancer are often treated with drugs (e.g., chemotherapeutics and antibiotics). In order to kill the cancer or diseased cells, the drug(s) must enter the cells and reach an effective dose so as to interfere with essential biochemical pathways. However, some cells evade being killed by the drug by developing resistance to it (termed “drug resistance”). Moreover, in some cases, cancer cells (also called tumor cells or neoplastic cells) develop resistance to a broad spectrum of drugs, including drugs that were not originally used for treatment. This phenomenon is termed “chemotherapeutic drug resistance.” There are different types of chemotherapeutic drug resistance, each associated with a different biological mechanism, and there are specific biological “markers” for different types of chemotherapeutic drug resistance that are clinically useful for detecting and diagnosing each type of resistance.

The emergence of the chemotherapeutic drug resistance, and also the multi-drug resistance (“MDR”), phenotype is the major cause of failure in the treatment of cancer (see, e.g., Davies (1994) Science 264: 375-382; Poole (2001) Cur. Opin. Microbiol. 4: 500-5008). The chemotherapeutic drug resistance phenotype can arise to a broad spectrum of functionally distinct drugs, whereby treatment options are significantly limited by chemotherapeutic drug resistance development. For that reason, the development of chemotherapeutic drug-resistant cancer cells is the principal reason for treatment failure in cancer patients (see Gottesman (2000) Ann. Rev. Med. 53: 615-627).

Chemotherapeutic drug resistance is multi-factorial. However, some mechanisms of resistance development are well known. For example, the classic MDR mechanism involves alterations in the gene for the highly evolutionarily conserved plasma membrane protein (P-glycoprotein or MDR 1) that actively transports or pumps drugs out of the cell or microorganism (Volm et al., (1993) Cancer 71: 3981-3987); Bradley and Ling, Cancer Metastasis Rev. (1994) 13: 223-233). Both human cancer cells and infectious bacterial pathogens may develop classic MDR via mechanisms involving the overexpression of P-glycoprotein due to amplification of the gene encoding P-glycoprotein. The overexpression of P-glycoprotein mRNA or protein in MDR cancer cells is a biological marker for MDR. Diagnostic tests and therapeutic methods have been developed that make use of the overexpression of P-glycoprotein marker to diagnose and to treat MDR cancer and pathogen infections (Szakacs et al., (1998) Pathol. Oncol. Res. 4: 251-257). However, because various normal tissues express different amounts of P-glycoprotein, there are significant problems with side effects as any therapy that targets P-glycoprotein on the cell surface of MDR cancer cells would also affect those normal tissues that also have a relatively high level of P-glycoprotein expression, such as liver, kidney, stem cells, and blood-brain barrier epithelium, the latter being a major contributor to the clinical side effects.

In addition to tumor growth and the development of chemotherapeutic drug resistance, metastasis of neoplastic cells to other sites in the body represents a serious impediment to successful treatment of cancer (see, e.g., Nomura and Katunuma (2005) J. Med. Invest. 52(1-2): 1-9). Metastasis is facilitated by proteinases that breakdown connective tissue, allowing neoplastic cells to slip into the blood stream and invade previously unaffected tissues. In particular, metalloproteinases and serine proteases (e.g., MMP-1 and MMP-9) are secreted into the extracellular environment for basal membrane breakdown (see Nomura and Katunuma (2005) J. Med. Invest. 52(1-2):1-9). Other proteases such as cathepsins B, L, and D are also utilized for connective tissue digestion, even though these enzymes are normally isolated intracellularly and are normally involved in lysosomal functions (see, e.g., Foekens et al. (1999) Br. J. Cancer 79: 300-307).

An important protease involved in tissue remodeling and neoplastic cell invasion is plasmin (see, e.g., Skrzydlewska et al. (2005) World J. Gastroenterol. 11(9): 1251-66). Cell surface receptors recruit plasminogen to a cell, and the enzyme is activated to yield the plasmin protease (see Skrzydlewska et al. (2005) World J. Gastroenterol. 11 (9): 1251-66). Increased plasmin located on the surface of tumor cells has been implicated in increased metastatic activity (see Rofstad et al. (2004) Cancer Res. 64(1): 13-8). Several potential plasminogen receptors have been identified, including the structural enzyme α-enolase (see Pancholi (2001) Cell Mol. Life Sci. 58(7): 902-20).

Enolase is an abundantly expressed glycolytic enzyme that catalyzes the dehydration of 2-phospho-D-glycerate into phosphoenolpyruvate, the second ATP production step in the glycolytic pathway (Wold et al. (1971) Toxicol. Appl. Pharmacol. 19(2):188-201). Three different enolase isoenzymes are found in vertebrates: α-enolase expressed in most tissues, β is muscle-specific, and γ is found only in nervous tissue. Distinct genes encode the three enolase isoforms, but their amino acid sequence shows remarkable phylogenetic conservation across species (Pancholi (2001) Cell Mol. Life Sci. 58(7): 902-20). All enolases form dimers composed of three distinct subunits encoded by the separate genes. The αα isoenzyme dimer is widely expressed in all fetal and adult mammalian tissues. The ββ enolase is found predominantly in muscle, whereas the γγ isoform is present in neurons and neuroendocrine tissues and has been frequently designated as neuron-specific enolase (NSE). The appearance of NSE is a late event in neural maturation, thus making it a useful marker of neuronal maturation. Developmental profile expression analysis of enolase isoforms during mammalian neuronal development has revealed the existence of a switch from the α to the γ isoform during neuronal differentiation (Marangos et al., (1980) Brain Res. 190(1):185-93).

α-enolase has also been shown to be an important structural enzyme in the lens of the human eye (see, e.g., Wistow et al. (1994) Biotechnol. Genet. Eng. Rev. 12:1-38). α-enolase has been found to localize to centromeres and microtubules in Hela cells (Johnstone et al. (1992) Exp. Cell. Res. 202(2):458-63). In addition, a shorter nuclear form of α-enolase also known as MBP-1 or Myc-promoter Binding Protein-1, is produced by an alternative translational initiation site located 400 bp downstream of the ATG (Feo et al. (2000) FEBS Lett. 473(1):47-52; Subramanian and Miller (2000) J. Biol. Chem. 275(8):5958-65). Furthermore, as a plasminogen receptor, α-enolase is involved in the regulation of tissue remodeling (see, e.g., Pancholi (2001) Cell Mol. Life Sci. 58(7): 902-20). These reports indicate a distinct role for the α-enolase isoform as a nuclear transcription factor implicated in oncogene regulation, cell growth control, tissue remodeling, and metastatic potential. It has also been shown that α-enolase expression appears to be upregulated in neoplastic cells relative to normal cells of the same tissue type. α-enolase is therefore a potential target for therapeutics directed at treating or preventing neoplastic development as well as preventing the development of chemotherapeutic drug resistance and metastatic potential.

There remains a need in both humans and animals for detecting, treating, preventing, and reversing the development of neoplastic cells. Furthermore, the need remains to detect, treat, prevent, and reverse the development of both classical and atypical MDR phenotypes in cancer cells and non-cancerous damaged cells. In addition, the ability to identify and to make use of reagents that identify multiple drug resistant cells has clinical potential for improvements in the treatment, monitoring, diagnosis, and medical imaging of chemotherapeutic drug-resistant cancer. There remains a need in both humans and animals for detecting, treating, preventing, and reversing the development of metastatic neoplastic cells in an organism. By facilitating clinical identification of neoplastic cells with metastatic potential, there is a potential for significant improvements in treatment of neoplastic cells.

### SUMMARY OF THE INVENTION

The present invention is based, in part, upon the discovery that α-enolase, a normal protein involved in carbohydrate metabolism, is expressed at high levels in neoplastic cells as compared to normal cells of the same tissue type, and at yet much higher levels in neoplastic cells that have developed chemotherapeutic drug resistance or metastatic potential. α-enolase expression levels are therefore diagnostic of neoplastic and chemotherapeutic drug resistant cancer cells. α-enolase expression is also diagnostic of metastatic potential. The invention provides a method that uses targeting agents specific for α-enolase to detect and diagnose neoplastic cells and cells with chemotherapeutic drug resistance and metastatic potential in neoplastic cells in a subject. Moreover, the invention provides therapeutic methods for treating neoplastic cells by increasing the sensitivity of the neoplastic cells to chemotherapeutic drugs. The invention also provides therapeutic methods for treating cells that have developed chemotherapeutic drug resistance or developed metastatic potential through the use of targeting agents specific for α-enolase.

In one aspect, the invention provides a method for diagnosing chemotherapeutic drug resistance in a neoplastic cell sample. The method comprises the detection of a level of α-enolase expressed in a neoplastic cell sample, and also the detection of a level of α-enolase in a non-resistant neoplastic cell. The method entails comparing the level of α-enolase expressed in the neoplastic cell sample to the level of α-enolase expressed in the non-resistant neoplastic cell of the same tissue type. Chemotherapeutic drug resistance is indicated if the level of α-enolase expressed in the neoplastic cell sample is greater than the level of α-enolase expressed in the non-resistant neoplastic cell of the same tissue type.

In another aspect, the invention provides a method of diagnosing a neoplastic cell. The method comprises detecting a level of expressed α-enolase in a test cell sample in which the test cell sample potentially contains a neoplastic cell from the group consisting of breast adenocarcinoma, small cell lung carcinoma, large cell lung carcinoma, lymphoblastic leukemia cells, chronic myelogeneous leukemia cells, acute promyelocytic leukemia cells, ovarian carcinoma, ovarian adenocarcinoma, and prostate adenocarcinoma. A level of α-enolase expressed in a normal cell of the same tissue type as the test cell sample is detected. The level of expressed α-enolase in the test cell sample is compared to the level of expressed α-enolase in the normal cell. The test cell sample is neoplastic if the level of α-enolase expressed in the test cell sample is greater than the level of α-enolase expressed in the normal cell sample.

In certain embodiments, the method entails detecting the levels of expressed α-enolase in the test cell sample, which comprises isolating a cytoplasmic sample from the test cell sample. In other embodiments, the enolase-targeting agent comprises an anti-α-enolase antibody or α-enolase-binding fragment thereof.

In certain embodiments, the level of expressed α-enolase in the test cell sample is detected by the method that comprises contacting the test cell sample with an α-enolase-targeting agent from the group including ligands, small molecules, nucleic acids, peptidomimetic compounds, inhibitors, peptides, proteins, and antibodies. In other embodiments, the level of antibody bound to α-enolase is detected by immunofluorescence, radiolabel, or chemiluminescence.

In particular embodiments, the method of detecting the level of expressed α-enolase in the cell comprises hybridizing a nucleic acid probe to a complementary α-enolase mRNA expressed in the test cell sample. In more particular embodiments, the method uses a nucleic acid probe from the group consisting of RNA, DNA, RNA-DNA hybrids, and siRNA. In still more embodiments, the level of α-enolase targeting agent is detected by labeling the targeting agent with a label including fluorophores, chemical dyes, radiolabels, chemiluminescent compounds, colorimetric enzymatic reactions, chemiluminescent enzymatic reactions, magnetic compounds, and paramagnetic compounds.

In certain embodiments, the test cell sample to be tested is isolated from a mammal. In other embodiments, the test cell sample to be tested is isolated from a human. In still other embodiments, the neoplastic cell is a breast adenocarcinoma. In yet other embodiments, the neoplastic cell is a lung carcinoma. In still more embodiments, the neoplastic cell is a lymphoblastic leukemia cell. In certain other embodiments, the test cell sample is isolated from a tissue from the group consisting of breast, skin, lymphatic, prostate, bone, blood, brain, liver, thymus, kidney, lung, and ovary.

In certain embodiments, the detection steps comprise detecting the level of a cell surface-expressed α-enolase in the test cell sample and in the normal cell sample. In other embodiments, the cell surface-expressed α-enolase is detected with an α-enolase targeting agent. In more embodiments, the cell surface-expressed α-enolase is detected with an α-enolase antibody or α-enolase-binding fragment thereof. In additional embodiments, the α-enolase targeting agent comprises plasminogen. In further embodiments, the α-enolase targeting agent comprises an inhibitor of α-enolase from the group consisting of phosphonoacetohydroxamate, (3-hydroxy-2-nitropropyl)phosphonate, (nitroethyl)phosphonate, and (phosphonoethyl)nitrolate. In particular embodiments, the α-enolase targeting agent is detected using a label including fluorophores, chemical dyes, radiolabels, chemiluminescent compounds, colorimetric enzymatic reactions, chemiluminescent enzymatic reactions, magnetic compounds, and paramagnetic compounds.

In another aspect, the invention provides a method of diagnosing chemotherapeutic drug resistance in a neoplastic cell. The method comprises detecting a level of expressed α-enolase in a potentially chemotherapeutic drug-resistant neoplastic cell sample from the group consisting of breast adenocarcinoma, small cell lung carcinoma, large cell lung carcinoma, lymphoblastic leukemia cells, chronic myelogeneous leukemia cells, acute promyelocytic leukemia cells, ovarian carcinoma, ovarian adenocarcinoma, and prostate adenocarcinoma. A level of α-enolase expressed in a non-chemotherapeutic drug-resistant neoplastic cell of the same tissue type as the potentially drug-resistant neoplastic cell sample is detected. The level of expressed α-enolase in the potentially drug-resistant neoplastic cell sample is compared to the level of expressed α-enolase in the non-drug-resistant neoplastic cell of the same tissue type. The potentially drug-resistant neoplastic cell sample is chemotherapeutic drug-resistant if the level of α-enolase expressed therein is greater than the level of α-enolase expressed in the non-chemotherapeutic drug-resistant neoplastic cell.

In certain embodiments, the method entails detecting the level of expressed α-enolase in the cell samples, which comprises isolating a cytoplasmic sample from the cell samples and measuring the level of α-enolase therein. In other embodiments, the method of detecting the level of expressed α-enolase in the neoplastic cell sample comprises contacting the cell samples with an anti-α-enolase antibody, or an α-enolase-binding fragment thereof, and detecting the level of antibody bound to α-enolase therein.

In some embodiments, the α-enolase targeting agent is from the group consisting of ligands, nucleic acids, synthetic small molecules, peptidomimetic compounds, inhibitors, peptides, and proteins. In other embodiments, the α-enolase targeting agent comprises plasminogen. In yet other embodiments, the α-enolase targeting agent comprises an inhibitor from the group consisting of phosphonoacetohydroxamate, (3-hydroxy-2-nitropropyl)phosphonate, (nitroethyl)phosphonate, and (phosphonoethyl)nitrolate. In still other embodiments, the method of detecting the levels of bound α-enolase targeting agent is performed using immunofluorescence, radiolabel, or chemiluminescence.

In particular embodiments, the method of detecting the level of expressed α-enolase in the cell samples comprises hybridizing a nucleic acid probe to a complementary α-enolase mRNA expressed in the cell samples. In more particular embodiments, the method uses a nucleic acid probe from the group consisting of RNA, DNA, RNA-DNA hybrids, and siRNA. In still more embodiments, the levels of nucleic acid probe hybridized to α-enolase mRNA is detected by a label from the group consisting of fluorophores, chemical dyes, radiolabels, chemiluminescent compounds, colorimetric enzymatic reactions, chemiluminescent enzymatic reactions, magnetic compounds, and paramagnetic compounds.

In certain embodiments, the potentially drug-resistant neoplastic cell sample is isolated from a mammal. In other embodiments, the potentially drug-resistant neoplastic cell sample is isolated from a human. In yet other embodiments, the potentially drug-resistant neoplastic cell sample is isolated from the group including breast, skin, lymphatic, prostate, bone, blood, brain, liver, kidney, lung, and ovary. In still other embodiments, the drug-resistant neoplastic cell sample comprises a breast adenocarcinoma that is resistant to a chemotherapeutic drug from the group consisting of taxol and adriamycin. In yet other embodiments, the drug-resistant neoplastic cell sample comprises an adriamycin-resistant lung carcinoma. In still more embodiments, the neoplastic cell sample comprises an adriamycin-resistant lymphoblastic leukemia cell. In particular embodiments, the non-resistant neoplastic cell sample comprises, or is derived from, a drug-sensitive cell line from the group consisting of MCF7, MDA, CEM, MOLT4, SKOV3, OVCAR3, 2008, PC3, CaOV3, HeLa, T84, HCT-116, and H69.

In some embodiments, the detection step comprises detecting the level of a cell surface-expressed α-enolase in the cell samples. In more embodiments, the surface-expressed α-enolase is detected with an α-enolase targeting agent. In other embodiments, the level of expressed α-enolase comprises isolating a membrane fraction from the neoplastic cell and detecting the level of cell surface expressed α-enolase in the membrane fraction. In still other embodiments, the α-enolase targeting agent is from the group consisting of antibodies, peptides, proteins, ligands, peptidomimetic compounds, and inhibitors. In further embodiments, the α-enolase targeting agent comprises an inhibitor from the group consisting of phosphonoacetohydroxamate, (3-hydroxy-2-nitropropyl)phosphonate, (nitroethyl)phosphonate, and (phosphonoethyl)nitrolate. In particular embodiments, the α-enolase targeting agent is detected using a label selected from the group consisting of fluorophores, chemical dyes, radiolabels, chemiluminescent compounds, colorimetric enzymatic reactions, chemiluminescent enzymatic reactions, magnetic compounds, and paramagnetic compounds.

In yet another aspect, the invention provides a method of diagnosing or detecting metastatic potential and/or angiogenic phenotype of a neoplastic cell sample. To determine the metastatic potential of a neoplastic cell sample, the method comprises detecting a level of expressed α-enolase in the potentially metastatic and/or angiogenic neoplastic cell sample. Also, the level of expressed α-enolase is detected in a nonmetastatic, nonangiogenic neoplastic cell sample of the same tissue type. The level of α-enolase expressed in the potentially metastatic and/or angiogenic neoplastic cell sample is then compared to the level of expressed α-enolase in a nonmetastatic, nonangiogenic neoplastic cell sample. Metastatic potential and/or angiogenic phenotype is indicated if the level of expressed α-enolase in the potentially metastatic and/or angiogenic neoplastic cell sample is greater than the level of expressed α-enolase in the nonmetastatic, nonangiogenic neoplastic cell sample.

In certain embodiments, the detection of the level of expressed α-enolase comprises detecting a level of cell surface-expressed α-enolase. In additional embodiments, detecting the level of expressed α-enolase comprises isolating a membrane fraction from the neoplastic cell and detecting the level of expressed α-enolase in the membrane fraction. In still other embodiments, detecting the level of cell surface expressed α-enolase comprises contacting the cell surface of the cell samples with an α-enolase targeting agent and detecting the level of α-enolase targeting agent bound to the cell surface of the cell samples.

In some embodiments, the α-enolase targeting agent is from the group consisting of ligands, synthetic small molecules, peptidomimetic compounds, inhibitors, peptides, proteins, and antibodies. In still other embodiments, the α-enolase targeting agent is an anti-α-enolase antibody or an α-enolase binding fragment thereof. In certain embodiments, the α-enolase targeting agent comprises plasminogen. In other embodiments, α-enolase targeting agent comprises an inhibitor of α-enolase from the group consisting of phosphonoacetohydroxamate, (3-hydroxy-2-nitropropyl)phosphonate, (nitroethyl)phosphonate, and (phosphonoethyl)nitrolate. In still further embodiments, the level of α-enolase targeting agent is detected by immunofluorescence, radiolabel, or chemiluminescence.

In certain embodiments, the potentially metastatic and/or angiogenic neoplastic cell sample is isolated from a mammal. In particular embodiments, the potentially metastatic and/or angiogenic neoplastic cell sample is isolated from a human. In more particular embodiments, the potentially metastatic and/or angiogenic neoplastic cell sample comprises a cell from the group consisting of a melanoma cell, a lymphoma cell, a sarcoma cell, a leukemia cell, a retinoblastoma cell, a hepatoma cell, a myeloma cell, a glioma cell, a mesothelioma cell, a adenocarcinoma cell, and a carcinoma cell. In still more particular embodiments, the potentially metastatic and/or angiogenic neoplastic cell sample is isolated from a tissue selected from the group consisting of breast, skin, lymphatic, prostate, bone, blood, brain, liver, thymus, kidney, lung, and ovary. In other embodiments, the nonmetastatic, nonangiogenic neoplastic cell sample comprises, or is derived from, a cell line including MCF7, CEM, MOLT4, OVCAR3, 2008, PC3, CaOV3, HeLa, T84, HCT-116, H69, HL60, H460, A549, K-562, SKOV3 and MDA-MB-231.

In still another aspect, the invention provides a method of treatment for a neoplasm in a patient. The method comprises administering an effective amount of an α-enolase targeting agent to the patient in which the targeting agent binds to α-enolase expressed in the neoplasm. The patient is administered an effective amount of a chemotherapeutic drug, whereby the α-enolase targeting agent, when bound to the neoplasm, increases the sensitivity of the neoplasm to the chemotherapeutic drug.

In some embodiments, the α-enolase targeting agent bound to the neoplasm is internalized into the neoplastic cell. In other embodiments, the α-enolase targeting agent comprises a liposome. In yet other embodiments, the liposome comprises a neoplastic cell-targeting agent on its surface. In more embodiments, the α-enolase targeting agent is from the group consisting of ligands, nucleic acids, synthetic small molecules, peptidomimetic compounds, inhibitors, peptides, proteins, and antibodies.

In more particular embodiments, the α-enolase targeting agent comprises an antibody or α-enolase binding fragment thereof. In certain embodiments, the neoplastic cell-targeting agent comprises an antibody, or α-enolase binding fragment thereof, specific for at least one cell marker from the group consisting of multidrug resistance protein 1, BRCP, p53, vimentin, α-enolase, nucleophosmin, and HSC70. In more embodiments, the α-enolase targeting agent comprises an inhibitor of α-enolase. In other embodiments, the inhibitor is from the group consisting of phosphonoacetohydroxamate, (3-hydroxy-2-nitropropyl)phosphonate, (nitroethyl)phosphonate, and (phosphonoethyl)nitrolate.

In some embodiments, the α-enolase targeting agent is a nucleic acid. In other embodiments, the nucleic acid is from the group consisting of RNA, DNA, RNA-DNA hybrids, and siRNA. In particular embodiments, the siRNA comprises 18 contiguous nucleotides of SEQ ID No: 2. In more particular embodiments, the siRNA comprises 25 contiguous nucleotides selected from the group consisting of SEQ ID No: 4 and SEQ ID No: 6.

In particular embodiments, the α-enolase targeting agent is administered to the patient by injection at the site of the neoplasm. In more particular embodiments, the α-enolase targeting agent is administered by surgical introduction at the site of the neoplasm. In still more particular embodiments, the α-enolase targeting agent is administered to the patient by inhalation of an aerosol or vapor.

In another aspect, the invention provides a kit for detecting a level of expression of α-enolase in a neoplastic cell sample. The kit comprises a first probe specific for α-enolase and a second probe for the detection of chemotherapeutic drug resistance. The second probe is specific for a marker from the group consisting of vimentin, multidrug resistance protein 1, BRCP, p53, HSC70, and nucleophosmin. A detection means for identifying a probe binding to a target is provided.

In certain embodiments, the first probe is from the group consisting of ligands, nucleic acids, synthetic small molecules, peptidomimetic compounds, inhibitors, peptides, proteins, and antibodies. In other embodiments, the first probe is a nucleic acid that is complementary to mRNA encoding α-enolase. In particular embodiments, the nucleic acid is from the group consisting of RNA, DNA, DNA-RNA hybrids, and siRNA.

In some embodiments, the first probe is an α-enolase-specific antibody or binding fragment thereof. In more particular embodiments, the first probe is plasminogen. In further embodiments, the first probe is from the group consisting of phosphonoacetohydroxamate, (3-hydroxy-2-nitropropyl)phosphonate, (nitroethyl)phosphonate, and (phosphonoethyl)nitrolate. In yet further embodiments, the second probe comprises a nucleic acid complementary to an mRNA encoding multidrug resistance protein 1, BRCP, p53, vimentin, HSC70, or nucleophosmin. In particular embodiments, the nucleic acid is from the group consisting of RNA, DNA, RNA-DNA hybrids, and siRNA. In certain embodiments, the second probe is an antibody or α-enolase binding fragment thereof. In still further embodiments, the kit utilizes a detection means from the group consisting of fluorophores, chemical dyes, radiolabels, chemiluminescent compounds, colorimetric enzymatic reactions, chemiluminescent enzymatic reactions, magnetic compounds, and paramagnetic compounds.

In still another aspect, the invention provides a pharmaceutical formulation for treating a neoplasm. The pharmaceutical formulation comprises an α-enolase-specific targeting component, a chemotherapeutic drug, and a pharmaceutically acceptable carrier. In certain embodiments, the pharmaceutical formulation comprises an α-enolase-specific targeting component, which includes ligands, nucleic acids, synthetic small molecules, peptidomimetic compounds, inhibitors, peptides, proteins, and antibodies.

In certain embodiments, the α-enolase-specific targeting component is a nucleic acid. In other embodiments, the nucleic acid is from the group consisting of RNA, DNA, RNA-DNA hybrids, and siRNA. In still other embodiments, the nucleic acid is a siRNA. In yet other embodiments, the siRNA comprises 18 contiguous nucleotides of SEQ ID NO: 2. In still other embodiments, the siRNA comprises 25 contiguous nucleotides selected from the group consisting of SEQ ID NO: 4 and SEQ ID NO: 6. In particular embodiments, the α-enolase-specific targeting component comprises an antibody or α-enolase binding fragment thereof.

In some embodiments, the α-enolase-specific targeting component comprises an inhibitor of α-enolase. In certain embodiments, the inhibitor is from the group consisting of phosphonoacetohydroxamate, (3-hydroxy-2-nitropropyl)phosphonate, (nitroethyl)phosphonate, and (phosphonoethyl)nitrolate.

In certain embodiments, the α-enolase-specific targeting component comprises a liposome. In particular embodiments, the liposome comprises a neoplastic cell-targeting component on its surface. In more particular embodiments, the pharmaceutical formulation includes a neoplastic cell-targeting component that comprises an antibody that binds to neoplastic cell-surface proteins from the group consisting of mutlidrug resistance protein 1, BRCP, p53, vimentin, α-enolase, nucleophosmin, and HSC70. In other embodiments, the neoplastic cell-targeting component comprises plasminogen.

In certain embodiments, the pharmaceutical formulation comprises a chemotherapeutic drug selected from the group consisting of Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, and Vinorelbine.

In another aspect, the invention provides a method of diagnosing a neoplastic cell. The method comprises detecting a level of α-enolase expressed in an ovarian cell sample. The ovarian cell sample potentially contains an ovarian cancer cell. The method further entails the detection of a level of α-enolase expressed in a normal ovarian cell sample. The level of expressed α-enolase in the ovarian cell sample is compared to the level of expressed α-enolase in the normal ovarian cell sample. A determination that the ovarian cell sample is neoplastic is made when the level of α-enolase expressed in the ovarian cell sample is greater than the level of α-enolase expressed in the normal ovarian cell sample.

### BRIEF DESCRIPTION OF THE FIGURES

The foregoing and other objects of the present invention, the various features thereof, as well as the invention itself may be more fully understood from the following description, when read together with the accompanying drawings in which:

FIG. 1A is a photographic representation of a 2D gel of silver stained MCF-7 extracts that shows the level of expression α-enolase protein.

FIG. 1B is a photographic representation of a 2D gel of silver stained MCF-7/AR extracts that shows the level of expression of α-enolase protein.

FIG. 2 is a chart showing the identity of peptides generated by tryptic digestion of a 44 kD spot isolated from MCF-7/AR 2D gels.

FIG. 3A is a photographic representation of an immunoblot probed with an anti-α-enolase antibody that shows the level of expression of α-enolase in drug-resistant and drug-sensitive MCF-7 and MDA cell extracts.

FIG. 3B is a photographic representation of an immunoblot probed with an anti-α-enolase antibody that shows the level of expression of α-enolase in drug-resistant (e.g., SKOV3 or normally sensitive cell lines that have developed resistance) and drug-sensitive OVCAR and 2008 cell extracts.

FIG. 3C is a photographic representation of an immunoblot probed with an anti-α-enolase antibody that shows the level of expression of α-enolase in drug-resistant (i.e., K562 or normally sensitive cell lines that have developed resistance) and drug-sensitive HSB-2, RPMI-8226, CEM, HL60, and MOLT4 cell extracts.

FIG. 3D is a photographic representation of an immunoblot probed with an anti-α-enolase antibody that shows the level of expression of α-enolase in drug-resistant and drug-sensitive H69 and H460 cell extracts.

FIG. 4A is a photographic representation of an immunoblot probed with an anti-α-enolase antibody that shows the level of expression of α-enolase in normal breast tissue and breast tumor.

FIG. 4B is a photographic representation of an immunoblot with an anti-α-enolase antibody that shows the level of expression of α-enolase in normal ovarian tissue and ovarian tumor.

FIG. 5 is a photographic representation of an immunoblot with an anti-α-enolase antibody that shows the level of expression of α-enolase in MCF-7 cells 3 days and 6 days after being treated with siRNA targeted to α-enolase.

FIG. 6 is a graphic representation of the results of an MTT cytotoxicity assay that shows the viability of α-enolase-depleted MCF-7 cells compared to controls.

FIG. 7A is a photographic representation of the results of an apoptosis assay of cells treated with α-enolase siRNA that shows MCF-7 and control cells when viewed by phase contrast microscopy.

FIG. 7B is a photographic representation of the results of an apoptosis assay of cells treated with α-enolase siRNA that shows MCF-7 and control cells when viewed by phase contrast microscopy.

FIG. 7C is a photographic representation of the results of an apoptosis assay of cells treated with α-enolase siRNA that shows MCF-7 and control cells stained with Annexin V FITC.

FIG. 7D is a photographic representation of the results of an apoptosis assay of cells treated with α-enolase siRNA that shows MCF-7 and control cells stained with Annexin V FITC.

FIG. 8A is a graphic representation of the results of an MTT cytotoxicity assay that shows the viability of α-enolase siRNA transfected MCF-7 cells treated with adriamycin compared to mock transfected MCF-7 controls.

FIG. 8B is a graphic representation of the results of an MTT cytotoxicity assay that shows the viability of α-enolase siRNA transfected MCF-7 cells treated with cisplatinum compared to mock transfected MCF-7 controls.

FIG. 8C is a graphic representation of the results of an MTT cytotoxicity assay that shows the viability of α-enolase siRNA transfected MCF-7 cells treated with vincristine compared to mock transfected MCF-7 controls.

FIG. 8D is a graphic representation of the results of an MTT cytotoxicity assay that shows the viability of α-enolase siRNA transfected MCF-7 cells treated with taxol compared to mock transfected MCF-7 controls.

FIG. 9A is a graphic representation of the results of an MTT cytotoxicity assay that shows the viability of α-enolase siRNA transfected CaOV3 cells treated with taxol compared to mock transfected CaOV3 controls.

FIG. 9B is a graphic representation of the results of an MTT cytotoxicity assay that shows the viability of α-enolase siRNA transfected CaOV3 cells treated with vinblastin compared to mock transfected CaOV3 controls.

FIG. 9C is a graphic representation of the results of an MTT cytotoxicity assay that shows the viability of α-enolase siRNA transfected CaOV3 cells treated with vincristine compared to mock transfected CaOV3 controls.

FIG. 10A is a graphic representation of the results of a clonogenic assay showing the effect of taxol treatment on MCF-7 cells transfected with α-enolase stealth siRNAs as compared controls transfected with mock siRNAs.

FIG. 10B is a graphic representation of the results of a clonogenic assay showing the effect of α-enolase depletion without chemotherapeutic drug treatment on cells transfected with α-enolase stealth siRNAs as compared controls transfected with mock siRNAs.

FIG. 11A is a photographic representation of an immunoblot probed with an anti-α- or enolase or anti-GAPDH antibody that shows the level of expression of α-enolase protein or GAPDH protein in A549 cells three days after mock transfection, transfection with a control vector, or transfection with a vector containing siRNA coding sequences targeting α-enolase.

FIG. 11B is a photographic representation of an immunoblot probed with an anti-α- or enolase or anti-GAPDH antibody that shows the level of expression of α-enolase protein or GAPDH protein in A549 cells six days after mock transfection, transfection with a control vector, or transfection with a vector containing siRNA coding sequences targeting α-enolase.

FIG. 12 is a graphic representation of the results of a MTT assay showing the effect of α-enolase siRNA treatment on A549 cells transfected with α-enolase siRNAs as compared to controls transfected with mock or control siRNAs.

FIG. 13A is a graphic representation of the results of a MTT cytotoxicity assay showing the effect of α-enolase siRNA treatment on the survival of A549 cells when challenged with docetaxel.

FIG. 13B is a graphic representation of the results of a MTT cytotoxicity assay showing the effect of α-enolase siRNA treatment on the survival of A549 cells when challenged with taxol.

FIG. 13C is a graphic representation of the results of a MTT cytotoxicity assay showing the effect of α-enolase siRNA treatment on the survival of A549 cells when challenged with vincristin.

FIG. 13D is a graphic representation of the results of a MTT cytotoxicity assay showing the effect of α-enolase siRNA treatment on the survival of A549 cells when challenged with vinblastin.

FIG. 13E is a graphic representation of the results of a MTT cytotoxicity assay showing the effect of α-enolase siRNA treatment on the survival of A549 cells when challenged with cisplatinum.

FIG. 13F is a graphic representation of the results of a MTT cytotoxicity assay showing the effect of α-enolase siRNA treatment on the survival of A549 cells when challenged with etoposide.

FIG. 13G is a graphic representation of the results of a MTT cytotoxicity assay showing the effect of α-enolase siRNA treatment on the survival of A549 cells when challenged with mitoxantrone.

FIG. 13H is a graphic representation of the results of a MTT cytotoxicity assay showing the effect of α-enolase siRNA treatment on the survival of A549 cells when challenged with doxorubicin.

FIG. 14 is a graphic representation of the results of a clonogenic assay showing the effect of α-enolase silencing on the survival of A549 cells treated with taxol or vincristin.

FIG. 15 is a photographic representation of an immunoblot probed with an anti-α-enolase antibody that shows the level of expression of α-enolase protein in H460 cells three and six days after transfection with a control siRNA or siRNA targeting α-enolase.

FIG. 16 is a graphic representation of the results of a MTT assay showing the effects of α-enolase siRNA silencing on the survival of H460 cells.

FIG. 17A is a graphic representation of the results of a MTT cytotoxicity assay showing the effect of α-enolase siRNA treatment on the survival of H460 cells when challenged with doxorubicin.

FIG. 17B is a graphic representation of the results of a MTT cytotoxicity assay showing the effect of α-enolase siRNA treatment on the survival of H460 cells when challenged with taxol.

FIG. 17C is a graphic representation of the results of a MTT cytotoxicity assay showing the effect of α-enolase siRNA treatment on the survival of H460 cells when challenged with vincristin.

FIG. 17D is a graphic representation of the results of a MTT cytotoxicity assay showing the effect of α-enolase siRNA treatment on the survival of H460 cells when challenged with docetaxel.

FIG. 17E is a graphic representation of the results of a MTT cytotoxicity assay showing the effect of α-enolase siRNA treatment on the survival of H460 cells when challenged with cisplatinum.

FIG. 17F is a graphic representation of the results of a MTT cytotoxicity assay showing the effect of α-enolase siRNA treatment on the survival of H460 cells when challenged with etoposide.

FIG. 17G is a graphic representation of the results of a MTT cytotoxicity assay showing the effect of α-enolase siRNA treatment on the survival of H460 cells when challenged with mitoxantrone.

FIG. 17H is a graphic representation of the results of a MTT cytotoxicity assay showing the effect of α-enolase siRNA treatment on the survival of H460 cells when challenged with vinblastin.

FIG. 18 is a photographic representation of an immunoblot probed with an anti-α-enolase antibody and anti-GAPDH antibody that shows the level of expression of α-enolase protein and GAPDH protein in SW-480 cells three and six days after transfection with mock siRNA, control siRNA, and siRNA targeting α-enolase.

FIG. 19A is a graphic representation of the results of a MTT cytotoxicity assay showing the effect of α-enolase siRNA treatment on the survival of SW-480 cells when challenged with taxol.

FIG. 19B is a graphic representation of the results of a MTT cytotoxicity assay showing the effect of α-enolase siRNA treatment on the survival of SW-480 cells when challenged with vincristin.

FIG. 20 is a photographic representation of an immunoblot probed with an anti-α-enolase antibody that shows the level of expression of α-enolase protein in MCF-7 cells transfected with the pCMV-ENO1 vector containing the cDNA coding for the full length human α-enolase.

FIG. 21A is a photographic representation of a phase contrast image showing the results of a cell adhesion assay involving H460 cells transfected with a mock vector.

FIG. 21B is a photographic representation of a phase contrast image showing the results of a cell adhesion assay involving H460 cells transfected with the pCMV-ENO1 vector.

FIG. 22A is a photographic representation of a phase contrast image showing the results of a cell adhesion assay involving MCF-7 cells transfected with a mock vector.

FIG. 22B is a photographic representation of a phase contrast image showing the results of a cell adhesion assay involving MCF-7 cells transfected with the pCMV-ENO1 vector.

FIG. 23A is a photographic representation of a phase contrast image showing the results of a cell adhesion assay involving A549 cells transfected with the pCMV-ENO1 vector.

FIG. 23B is a photographic representation of a phase contrast image showing the results of a cell adhesion assay involving A549 cells transfected with the pCMV-ENO1 vector.

FIG. 24 is a graphic representation of the results of a cell adhesion assay showing the effect of α-enolase silencing on the adhesion of A549 and MCF-7 cells to a laminin-coated multi-well plate.

FIG. 25 is a graphic representation of the results of a cell adhesion assay showing the effect of α-enolase silencing on the adhesion of A549 cells to a collagen-coated multi-well plate.

FIG. 26A is a graphic representation of the results of a cell adhesion assay showing the effect of α-enolase silencing (cells transfected with Eno-1 siRNA) or overexpression (cells tranfected with pCMV-Eno-1) on the adhesion of A549 and MCF-7 cells as compared to mock transfectants or cells transfected with a control vector.

FIG. 26B is a graphic representation a comparison of the results of a cell adhesion assay showing the effect of α-enolase silencing (cells transfected with Eno-1 siRNA) or overexpression (cells tranfected with pCMV-Eno-1) on the adhesion of A549 and MCF-7 cells as compared to mock transfectants.

FIG. 27 is a graphic representation of the results of a cell invasion transwell filter assay showing the effect of α-enolase silencing (cells transfected with Eno-1 siRNA) or overexpression (cells tranfected with pCMV-Eno-1) on the adhesion of A549 cells as compared to control cells.

FIG. 28 is a photographic representation of an immunoblot probed with an anti-α-enolase antibody and anti-GAPDH antibody showing the level of expression of α-enolase protein and GAPDH protein in MDA-MB-435 breast cancer cells three and six days after a mock transfection, transfection with a control vector, or transfection with a vector expressing α-enolase

FIG. 29 is a graphic representation of the results of a cell invasion transwell filter assay showing the effect of α-enolase silencing on the metastatic potential of MDA-MB-435 breast cancer cells as compared to control cells.

FIG. 30 is a photographic representation of an immunoblot probed with an anti-α-enolase antibody and anti-GAPDH antibody that shows the level of expression of α-enolase protein and GAPDH protein in MCF-7 cells three days after transfection with control siRNA and siRNA to VEGF.

FIG. 31 is a photographic representation of an immunoblot probed with an anti-α-enolase antibody and anti-GAPDH antibody that shows the level of expression of α-enolase protein and GAPDH protein in HUVEC cells 2 days after transfection with a vector expressing α-enolase siRNA.

FIG. 32A is a photographic representation of a phase contrast image of a capillary tube formation assay showing the ability of HUVEC cells transfected with control siRNA to form capillary tubes on Matrigel.

FIG. 32B is a photographic representation of a phase contrast image of a capillary tube formation assay showing the effect of α-enolase silencing on the ability of HUVEC cells to form capillary tubes on Matrigel.

FIG. 33A is a graphic representation of the results of a mRNA expression analysis showing the difference in the levels of α-enolase mRNA expression in normal ovarian tissue and ovarian tumors.

FIG. 33B is a graphic representation of the results of a protein expression analysis that shows the difference in the levels of α-enolase protein expression in normal ovarian tissue and ovarian tumors.

FIG. 34 is a graphic representation demonstrating the difference in α-enolase mRNA expression in ovarian cancer tumor cells from cancer patients compared to normal ovarian tissue.

FIG. 35 is a graphic representation demonstrating the difference in α-enolase mRNA expression in lung cancer tumor cells from cancer patients compared to normal lung tissue.

FIG. 36 is a graphic representation demonstrating the difference in α-enolase mRNA expression in breast cancer tumor cells from cancer patients compared to normal breast tissue.

FIG. 37 is a schematic representation of the nucleotide sequence of human α-enolase [SEQ ID NO: 10].

FIG. 38A is a photographic representation of the effects of α-enolase expression on the growth and morphology of MCF-7 breast tumor cells by transfection with Control.

FIG. 38B is a photographic representation of the effects of α-enolase expression on the growth and morphology of MCF-7 breast tumor cells by transfection with Eno-1 S siRNA.

FIG. 38C is a photographic representation of the effects of α-enolase expression on the growth and morphology of MCF-7 breast tumor cells by transfection with Eno-2 S siRNA.

FIG. 39 is a graphic representation of the effect of α-enolase expression on cell proliferation of MCF-7 and MDA-435 breast cancer cells respectively following MOCK, Control S, or Eno-1S siRNA transfection.

FIG. 40 is a graphic representation of the effect of α-enolase down-regulation on the viability of MDA-435 breast cancer cells following MOCK, siGLO, Enolase-1, Control S and Enolase-1 S siRNA transfection.

FIG. 41 is a graphic representation of the effect of α-enolase silencing in MDA-435 cells following MOCK transfection or transfection with Control St, Eno-1 St, Rhodamine or Eno-1α-enolase siRNA for 3 days (left side of panel) or 6 days (right side of panel).

FIG. 42A is a photographic representation of the viability of MDA-435 breast cancer cells as determined by annexin V FITC staining following MOCK transfection.

FIG. 42B is a photographic representation of the viability of MDA-435 breast cancer cells as determined by annexin V FITC staining following transfection with Control St siRNA.

FIG. 42C is a photographic representation of the viability of MDA-435 breast cancer cells as determined by Annexin V FITC staining following transfection with Eno-1 S siRNA.

FIG. 43 is a graphic representation of the effect of α-enolase down-regulation on DNA synthesis in MDA-435 breast cancer cells as determined by BrdU incorporation following MOCK transfection or transfection with siControl or Eno-1 siRNA.

FIG. 44A is a photographic representation of capillary tube formation (angiogenesis) in HUVEC treated with taxol.

FIG. 44B is a photographic representation of capillary tube formation (angiogenesis) in HUVEC treated with taxol and down-regulated of α-enolase expression.

FIG. 44C is a photographic representation of capillary tube formation (angiogenesis) in mock-transfected HUVEC cells.

FIG. 44D is a photographic representation of capillary tube formation (angiogenesis) in HUVEC transfected with α-enolase siRNA.

FIG. 45 is a graphic representation of the downregulation of α-enolase protein in PC-3 prostate cancer cells following MOCK transfection or transfection with Eno-1 St or Eno-1 siRNA for three (3) or six (6) days as determined by α-enolase specific monoclonal antibody

FIG. 46A is a graphic representation of the results of an MTT cytotoxicity assay showing the viability of α-enolase siRNA-transfected PC-3 prostate cancer cells treated with taxol compared to control St transfected PC-3 cells treated with the same.

FIG. 46B is a graphic representation of the results of an MTT cytotoxicity assay showing the viability of α-enolase siRNA-transfected PC-3 prostate cancer cells treated with docetaxel compared to control transfected PC-3 cells treated with the same.

FIG. 46C is a graphic representation of the results of an MTT cytotoxicity assay showing the viability of α-enolase siRNA-transfected PC-3 prostate cancer cells treated with vincristin compared to control PC-3 cells treated with the same.

FIG. 46D is a graphic representation of the results of an MTT cytotoxicity assay that shows the viability of α-enolase siRNA-transfected PC-3 prostate cancer cells treated with vinblastin compared to control PC-3 cells treated with the same.

FIG. 47 is a photographic representation of an immunoblot showing the levels of expression of α-enolase protein at three days and six days post-transfection in Detroit 551 normal skin fibroblast cells transfected with mock control, control siRNA, Eno-1 siRNA targeting α-enolase, and Eno-1 stealth siRNA targeting α-enolase.

FIG. 48 is a graphic representation showing the viability of Detroit 551 normal skin fibroblast cells three days post-transfection with mock control, control siRNA, Eno-1 siRNA targeting α-enolase, and Eno-1 siRNA targeting α-enolase.

FIG. 49 is a photographic representation showing the viability of HFL-1 normal lung fibroblast cells three days post-transfection with mock control, control siRNA, Eno-1 siRNA targeting α-enolase, and Eno-1 siRNA targeting α-enolase.

FIG. 50 is a photographic representation showing the viability of MRC-5 fetal lung fibroblast cells three days post-transfection with mock control, control siRNA, Eno-1 siRNA targeting α-enolase, and Eno-1 siRNA targeting α-enolase.

### DETAILED DESCRIPTION OF THE INVENTION

The patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

1.1 General

Aspects of the present invention provide methods and reagents for detecting, diagnosing, preventing, and treating the development of cancer in a patient. In some aspects, the neoplasm is made more sensitive to the chemotherapeutic treatment by decreasing the level of expression of α-enolase in the neoplastic cells. Furthermore, aspects of the present invention provide methods and reagents for detecting and diagnosing chemotherapeutic drug-resistant cancer, and for detecting and diagnosing metastatic cancers. Other aspects of the invention provide methods and reagents to treat and/or prevent the development of metastatic and/or chemotherapeutic drug-resistant cancer in a patient. Additionally, the invention allows for the improved clinical identification and treatment of patients having chemotherapeutic drug-resistant and/or metastatic tumors.

Accordingly, the invention provides, in part, methods for diagnosing a neoplastic cell in a patient. One method of the present invention includes measuring a level of expression of α-enolase in a cell sample and comparing the level of expression of α-enolase in the cell sample to the level of expression of α-enolase in a normal cell of the same tissue type. If the level of expression of α-enolase is greater in the cell sample than in the normal cell, a neoplastic cell is indicated. In some embodiments, the neoplastic cell sample and the normal cell are separated into fractions, and the cytoplasmic fractions are tested for α-enolase expression.

The invention further provides methods for diagnosing the development of chemotherapeutic drug resistance in a neoplastic cell. One method of the present invention includes measuring a level of expression of α-enolase in a neoplastic cell sample and comparing the level of expression of α-enolase in the neoplastic cell sample to the level of expression of α-enolase in a non-resistant neoplastic cell of the same tissue type. If the level of expression of α-enolase is greater in the neoplastic cell sample than in the non-resistant neoplastic cell, chemotherapeutic drug-resistance is indicated. In some embodiments, the neoplastic cell sample and the non-resistant neoplastic cell are separated into fractions, and the cytoplasmic fractions are tested for α-enolase expression.

As used herein, the term “derived from” means to obtain from a source. In the case of cells, cells may be obtained from any source whether it be an organisms such as a mammal or cells maintained outside of the organism. For example, a neoplastic cell can be derived from cell lines including, but not limited to, MCF-7, MDA, SKOV3, Molt-4, CEM, MOLT4, OVCAR3, 2008, PC3, CaOV3, HeLa, T84, HCT-116, H69, HL60, H460, A549 and K-562.

As used herein, a “neoplastic cell” is a cell that shows aberrant cell growth, such as increased, uncontrolled cell growth. A neoplastic cell can be a hyperplastic cell, a cell from a cell line that shows a lack of contact inhibition when grown in vitro, a tumor cell when grown in vivo, or a cancer cell that is capable of metastasis in vivo. Alternatively, a neoplastic cell can be termed a “cancer cell.” Non-limiting examples of cancer cells include melanoma, breast cancer, ovarian cancer, lung cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma, and lymphoma cells, melanoma cells, sarcoma cells, leukemia cells, retinoblastoma cells, hepatoma cells, myeloma cells, glioma cells, mesothelioma cells, and carcinoma cells.

Cancer cells can be obtained from non-limiting tissues such as breast, lung, bone, blood, skin, brain, gastrointestinal, lymphatic, hepatic, muscle, ovary, uterine, and kidney. Cancer cells can be obtained from tissues other than the tissue from which the cancer cell originally developed, as in the case of metastasized cancer cells. Moreover, cancer cells can be obtained from mammals including, but not limited to, human, non-human primates such as chimpanzee, mouse, rat, guinea pig, chinchilla, rabbit, pig, and sheep.

Alternatively, cancer cells can be obtained in the form of a cell line. The term “cell line”, as used herein, refers to any cell that has been isolated from the tissue of a host organism and propagated by artificial means outside of the host organism. Such cell lines can be chemotherapeutic drug-resistant or chemotherapeutic drug-sensitive. A cell line is isolated and derived from tissues such as prostatic tissue, bone tissue, blood, brain tissue, lung tissue, ovarian tissue, epithelial tissue, breast tissue, and muscle tissue. A cell line can be derived, produced, or isolated from a cancer cell type, e.g., melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, or thymoma. Cell lines can also be generated by techniques well known in the art (see, e.g., Griffin et. al., (1984) Nature 309(5963): 78-82). Useful, exemplary, and non-limiting cell lines include MCF7, MDA, SKOV3, OVCAR3, 2008, PC3, T84, HCT-116, H69, H460, HeLa, MOLT4, CEM, CaOV3, HL60, A549 and K-562.

As used herein, the term “normal cell” means a cell that exhibits the characteristics expected for a cell of its particular tissue type, age, developmental stage, and organism. A normal cell generally exhibits growth characteristics that are not aberrant when compared to the cells of its particular tissue type, age, developmental stage, and organism. Normal cells do not tend to harm the functionality of the tissue from which they are isolated. In addition, normal cells do not show uncontrolled growth within the organism. “Uncontrolled growth” can generally be defined as growth that is outside the normal range exhibited by cells of a particular tissue type, age, developmental stage, and organism.

Normal cells are used to establish the baseline levels of expression of α-enolase for a particular tissue or cell type. Normal cells are used as a comparison tissue so that a physician can determine whether a particular cell sample is neoplastic. Normal cells can be obtained from healthy sources such as subjects not presently suffering from a neoplasm. It should be noted, however, that the subjects should not have been previous cancer patients. Normal tissues can be obtained from tissue banks such as the CORE Tissue Bank at the University of California at Los Angeles (Los Angeles, Calif.) and the University of British Columbia (Vancouver, BC, CA). Alternatively, normal tissues can be obtained using methods known in the art (see, e.g., Villalba et al. (2001) Cell Tissue Bank. 2(1): 45-49; Jewell et al. (2002) Am. J. Clin. Pathol. 18(5): 733-41).

As used herein, the term “nonmetastatic” refers to the inability of a neoplastic cell to spread from an organ or tissue of origin to another part of the body. Nonmetastatic neoplastic cells do not exhibit tissue invasiveness or a decrease in cell adhesion with basement membranes.

As used herein, “nonangiogenic” relates to the inability of a neoplastic cell to regulate the proliferation of new blood vessels under physiological conditions. Nonangiogenic neoplastic cells are incapable of inducing the growth of new blood vessels into tumor tissues or generating aberrant vascularization of tissues.

As used herein, the term “metastatic potential” means the ability of a neoplastic cell to spread from tissue or organ of origin to another tissue or organ in the body of the subject. Metastatic potential is a stage of tumor progression. As used herein, “tumor progression” refers to the process by which cells evolve from a benign state to a malignant state. A “malignant state” occurs when a tumor or neoplastic cell exhibits one or more of the following characteristics: 1) self-sufficient growth, 2) insensitivity to growth inhibition, 3) evasion of apoptotic signals, 4) immortalization, 5) angiogenic potential, and 6) metastatic potential. A malignant tumor or neoplastic cell typically shows aggressive behavior, and has the tendency to invade local tissues or metastasize to more distant tissues.

As used herein, “chemotherapeutic drug” means a pharmaceutical compound that kills a damaged cell such as a cancer cell. Cell death can be induced by the chemotherapeutic drug through a variety of means including, but not limited to, apoptosis, osmolysis, electrolyte efflux, electrolyte influx, cell membrane permeablization, and DNA fragmentation. Exemplary non-limiting chemotherapeutic drugs are adriamycin, cisplatinum, taxol, melphalan, daunorubicin, dactinomycin, bleomycin, fluorouracil, teniposide, vinblastin, vincristine, methotrexate, mitomycin, docetaxel, chlorambucil, carmustine, mitoxantrone, and paclitaxel.

As used herein, the term “chemotherapeutic drug-resistance” encompasses the development of resistance to a particular chemotherapeutic drug, class of chemotherapeutic drugs or multiple chemotherapeutic drugs by a cancer cell. Resistance can occur before or after treatment with a chemotherapy regime. Without being limited to any one theory, the mechanism of development of chemotherapeutic drug resistance can occur by any means, such as by pathogenic means such as through infections, particularly viral infection. Alternatively, chemotherapeutic drug resistance can be conferred by a mutation or mutations in one or several genes located either chromosomally or extrachromosomally. In addition, chemotherapeutic drug resistance can be conferred by selection of a certain phenotype by exposure to the chemotherapeutic drug or class of chemotherapeutic drugs, and then subsequent survival of the cell to the particular treatment. The above-mentioned mechanisms of chemotherapeutic drug resistance are known in the art. The terms, “chemotherapeutic drug-resistant” and “chemotherapeutic drug resistance,” are used to describe a neoplastic cell or a damaged cell that is chemotherapeutic drug-resistant due to either the classical mechanism (i.e., involving P-glycoprotein or another MDR protein) or an atypical mechanism (non-classical mechanism) that does not involve P-glycoprotein (e.g., an atypical mechanism that involves the MRP1 chemotherapeutic drug resistance marker).

As used herein, the term “MDR protein” includes any of several integral transmembrane glycoproteins of the ABC type that are involved in (multiple) drug resistance. These include MDR 1 (P-glycoprotein or P-glycoprotein 1), an energy-dependent efflux pump responsible for decreased drug accumulation in chemotherapeutic drug-resistant cells. Examples of MDR 1 include human MDR 1 (see, e.g., database code MDR1_HUMAN, GenBank Accession No. P08183, 1280 amino acids (141.34 kD)). Other MDR proteins include MDR 3 (or P-glycoprotein 3), which is an energy-dependent efflux pump that causes decreased drug accumulation but is not capable of conferring drug resistance by itself. Examples of MDR 3 include human MDR 3 (see, e.g., database code MDR3_HUMAN, GenBank Accession No. P21439, 1279 amino acids (140.52 kD). Other MDR-associated proteins participate in the active transport of drugs into subcellular organelles. Examples from human include MRP 1, Chemotherapeutic drug Resistance-associated Protein 1, database code MRP_HUMAN, GenBank Accession No. P33527, 1531 amino acids (171.47 kD).

In some embodiments of the invention, targeting agents are used to detect the level of expression of α-enolase in a cell sample. As used herein, the term “targeting agent” means a compound that can bind, associate, or hybridize with a target molecule in a specific manner. The mechanisms of binding to a target molecule include, e.g., hydrogen bonding, Van der Waals attractions, covalent bonding, ionic bonding, or hydrophobic interactions. In certain embodiments, a targeting agent is used to detect the level of expression of α-enolase in a neoplastic cell sample. Non-limiting examples of targeting agents include antibodies, antibody fragments, inhibitors of α-enolase, nucleic acids, proteins, peptides, and peptidomimetic compounds.

As used herein, the term “α-enolase targeting agent” refers to compounds that can specifically bind to α-enolase expressed in the cell. α-enolase can be expressed as a nucleic acid such as messenger RNA (“mRNA”) that encodes for α-enolase polypeptide or a fragment of the polypeptide. Also, α-enolase can be expressed as a polypeptide or as fragments of the completed polypeptide. Targeting agents include, but are not limited to, compounds such as antibodies or fragments thereof, peptides, peptidomimetic compounds, nucleic acids, and small molecules.

As used herein, the term “inhibitor” means a compound that prevents a biomolecule, e.g., a protein, nucleic acid, or ribozyme, from completing a reaction. An inhibitor can inhibit a reaction by competitive, uncompetitive, or non-competitive means. Exemplary inhibitors include, but are not limited to, nucleic acids, proteins, small molecules, chemicals, peptides, peptidomimetic compounds, and analogs that mimic the binding site of an enzyme. In some embodiments, the inhibitor can be nucleic acid molecules including, but not limited to, siRNA that reduce the amount of functional protein in a cell.

Inhibitors of α-enolase include non-limiting competitive inhibitors such as phosphonoacetohydroxamate, (3-hydroxy-2-nitropropyl)phosphonate, (nitroethyl)phosphonate, and (phosphonoethyl)nitrolate. Useful inhibitors are compounds that bind to α-enolase and reduce the “effective activity” of α-enolase in a cell or cell sample. Compounds that reduce the effective activity of α-enolase through binding to sites other than enzymatic region of α-enolase include, but are not limited to, antibodies, antibody fragments such as “Fv,” “F(ab′)2,” “F(ab),” “Dab” and single chains representing the reactive portion of an antibody (“SC-Mab”), peptides, peptidomimetic compounds, and small molecules (see, e.g., Lopez-Alemany et al (2003) Am. J. Hematol. 72(4): 234-42; Miles et al. (1991) Biochem. 30(6): 1682-91). The term “effective activity” as used herein refers to a protein's ability to perform a specific function at a level to produce a phenotype such as chemotherapeutic drug resistance. In addition, peptides that interact with the C-terminal lysine residues of α-enolase can be inhibitors of plasminogen binding activity. Exemplary inhibitors include, but are not limited to, peptides containing carboxy-terminal lysyl residues such as the carboxy-terminal 19 amino acids of α-2-antiplastin and lysine derivatives with free α-carboxyl groups (see, e.g., Miles et al. (1991) Biochem. 30(6): 1682-91).

Aspects of the present invention provide methods of diagnosing a neoplasm in a patient. The methods include administering to a cancer patient an α-enolase targeting agent and detecting the α-enolase targeting agent that is bound to expressed α-enolase using a detectable label operably linked to the α-enolase targeting agent. In addition, specific aspects of the present invention provide methods of detecting chemotherapeutic drug resistance in a patient. The methods include administering to a cancer patient an α-enolase targeting agent and detecting the α-enolase targeting agent that is bound to expressed α-enolase using a detectable label operably linked to the α-enolase targeting agent.

Aspects of the present invention also allow the identification of those patients whose neoplastic cells have acquired chemotherapeutic drug resistance. In some situations, the patient is identified when he/she no longer responds to the drug being used in his/her treatment. For example, a breast cancer patient in remission being treated with a chemotherapeutic agent (e.g., vincristine) may suddenly come out of remission, despite being constantly treated with the chemotherapeutic agent. Unfortunately, such a patient is often found also to be unresponsive to other chemotherapeutic agents, including some to which the patient has never been exposed. Of course, after these patients become chemotherapeutic drug-resistant, treating these patients to control their now-resurgent cancer or disease caused by a damaged cell is difficult and may require more drastic therapies, such as radiotherapy or surgery (e.g., bone marrow transplantation or amputation of necrotic tissue).

Some aspects of the present invention also allow an early diagnosis of neoplasms and/or neoplasms that have developed chemotherapeutic drug resistance by detecting increased amounts of α-enolase in neoplastic cells of the patient. In certain instances, early diagnosis of a neoplasm will improve the odds of survival for a patient by diagnosing and treating the neoplasm before it metastasizes or develops chemotherapeutic drug resistance. For neoplasms that have developed chemotherapeutic drug resistance, an early diagnosis allows patients who are initially drug responders and sensitive to drug treatment to be distinguished from those who are initially drug non-responders. Further, diagnostic procedures using α-enolase expression may also be used to follow the development and emergence of MDR neoplastic cells that are resistant to the treatment drug and that arise during the course of drug treatment, permitting health professionals to tailor their treatments accordingly.

Furthermore, the invention provides a method of screening for a neoplasm in a subject potentially harboring the neoplasm. Patients can be administered a detectably labeled α-enolase targeting agent operably linked to a detectable label. Once the α-enolase targeting agent is administered, the physician can detect the presence of the α-enolase targeting agent by means s including, but not limited to, MRI and CAT scan. The binding of the α-enolase targeting agent to a particular group of cells or tissue will indicate the potential presence of a neoplasm, which may be chemotherapeutic resistant and/or angiogenic. The physician can determine that a particular tissue is expressing above normal levels of α-enolase by comparing the image to other images from previously examined patients that had no neoplasm present in a particular tissue. The physician's experience can be used to determine whether a particular tissue is expressing above normal levels of α-enolase, thereby increasing the likelihood that the tissue is neoplastic.

The α-enolase targeting agent can be specifically targeted to a neoplasm. To target the α-enolase targeting agent, the agent can be incorporated into a liposome formulation, which can be an immunoliposome.

Typically, the α-enolase targeting agent is targeted to the neoplasm orally, subcutaneously, transdermally, surgically, or intravenously. The α-enolase targeting agent includes, but is not limited to, compounds such as ligands, synthetic small molecules, nucleic acids, peptidomimetic compounds, inhibitors, peptides, proteins, and antibodies. The α-enolase targeting agent can be an antibody or binding fragment thereof. It should be noted that the nucleic acids can include, but are not limited to, DNA, RNA, RNA-DNA hybrids, siRNA, and aptamers. Moreover, the detectable label can be any label so long as the label does not affect the targeting function of the α-enolase targeting agent. Labels include, but are not limited to, fluorophores, chemical dyes, radiolabels, chemiluminescent compounds, colorimetric enzymatic reactions, chemiluminescent enzymatic reactions, magnetic compounds, and paramagnetic compounds.

In addition, diagnostic assays for α-enolase cell surface expression are useful for selecting patients in clinical studies having metastatic neoplastic cells. Hence, the presence of α-enolase on the cell surface of neoplastic cells of a patient identifies the patient as having the potential for metastatic development, thereby allowing for alternative or additional prophylactic treatment and also for inclusion or exclusion from clinical studies. Diagnostic assays can also identify patients with tumors or neoplastic cells capable of angiogenesis. Angiogenesis has been linked previously with increased tumor aggressiveness and metastatic potential. This appears to be due to the present finding that α-enolase is secreted from certain tumor cells that have increased expression of this protein. For that reason, increased expression of α-enolase indicates that the cell may be predisposed to inducing vascular development, which increases the likelihood that more aggressive treatments should be undertaken. In certain aspects of the invention, α-enolase activity can be blocked by antibodies targeted to the cell surface of certain tumor cells, blocking the angiogenic and metastatic effects of increased α-enolase expression. Furthermore, patients with the potential of chemotherapeutic drug resistance can be identified by detecting the overexpression of α-enolase in a neoplasm. This information can also be used to determine a patient's suitability for a particular treatment or for inclusion in certain clinical studies.

The invention further provides methods for diagnosing or detecting tumor progression in a cancer cell sample. A method includes measuring a level of expressed α-enolase in a neoplastic cell sample and comparing the level of expressed α-enolase in the neoplastic cell sample to the level of expressed α-enolase in a non-resistant neoplastic cell of the same tissue type. If the level of expression of α-enolase is greater in the neoplastic cell sample than in the non-resistant neoplastic cell, tumor progression is indicated. In some embodiments, the neoplastic cell sample and the non-resistant neoplastic cell can be separated into fractions, and the cell membrane fractions can be tested for the levels of cell surface expressed α-enolase.

As used herein, the term “metastatic” relates to the spreading of neoplastic cells from a primary site of growth to other sites in an organism. Cells grown ex vivo show metastatic potential through their ability to grow without contacting a support or matrix.

Metastatic cells or cells with metabolic potential can be isolated from non-limiting tissues such as breast, ovarian, bone, muscle, gastrointestinal such as stomach and intestine, hepatic, kidney, heart, lung, brain, skin, blood, lymphatic, and mucosal. Metastatic cells can also be derived from cancer cell types including, but not limited to, adenocarcinoma, melanoma, breast cancer, ovarian cancer, lung cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma, and lymphoma cells, melanoma cells, sarcoma cells, leukemia cells, retinoblastoma cells, hepatoma cells, myeloma cells, glioma cells, mesothelioma cells, and carcinoma cells.

In some methods of the invention, a membrane fraction is isolated from a neoplastic cell sample prior to being contacted with an α-enolase targeting agent. The membrane fraction can be isolated using techniques known in the art. For instance, cell lysis can be accomplished by non-limiting techniques such as osmolysis, sonication, lysis by pressure means, or grinding of the cells by dounce. Cell lysis is typically followed by differential separation of cellular components using procedures known in the art (see, e.g., Neville (2005) J. Biophys. Biochem. Cytol. 8: 413-422). Purified membranes can be contacted with various α-enolase targeting agents for the purpose of detecting α-enolase expression. These targeting agents include, but are not limited to, anti-α-enolase antibodies or binding fragments thereof, inhibitors of α-enolase, and plasminogen. Small molecules, peptides, and peptidomimetic compounds can also be used so long as these compounds show specific binding or association with α-enolase. In addition, these compounds can be labeled for the purposes of detection as described below.

The invention also provides methods of treating or preventing the growth of a chemotherapeutic drug-resistant neoplasm in a patient. The methods include administering an effective amount of α-enolase targeting agent to a patient, the targeting agent being targeted to the neoplasm or to a site in close proximity to the neoplasm. Treatment of the patient includes administering a chemotherapeutic drug to kill the neoplastic cells after the cells have been targeted by the α-enolase targeting agent to reduce or prevent the chemotherapeutic drug resistance of the neoplastic cells. Alternatively, the targeting agent and the chemotherapeutic drug can be administered simultaneously, e.g., as a single, linked therapeutic.

The α-enolase targeting agent can be composed of multiple parts, herein termed “components.” For example, the α-enolase targeting agent can have a cell-associating component. A useful cell-associating component is an antibody or binding fragment of an antibody such as Fv, F(ab′)₂, F(ab), Dab, and SC-Mab that binds to cell surface expressed cancer cell markers such as Pgp-1, multidrug resistance protein 1 (“MRP1”), BIP, BRCP, HSC70, nucleophosmin, vimentin, and HSP90. The cell-associating component can also be a compound that binds to a cell marker such as, but not limited to, an inhibitor of a cancer cell marker, a peptide, a peptidomimetic, a ligand, or a small molecule. As long as the interaction of the cell-associating component allows for cancer cell-specific targeting of the α-enolase targeting agent, a compound is useful as a cell-associating component. The α-enolase targeting agent also can include a cell-internalization component that allows the α-enolase targeting agent to enter into the cell. For example, a cell-internalization component can be an agent that allows for cell membrane fusion between the α-enolase targeting agent and the cancer cell, such as a liposome or immunoliposome (see, e.g., Drummond, et al, (2005) Ann. Rev. Pharmacol. Toxicol. 45: 495-528).

The cell-internalization component can be a dendrimer conjugate, which is a spherical polymer (see, e.g., Tomalia, D. A., et al., (1990) Angew. Chem. Int. Ed. Engl. 29: 5305). Synthesis and utilization of dendrimers has been postulated in the art, and dendrimers have been utilized for chemotherapeutic drug targeting in vitro (see, e.g., P. Singh, et al., (1994) Clin. Chem. 40: 1845). The α-enolase-specific targeting component should bind to α-enolase or a portion of α-enolase so as to decrease the effective activity of the enzyme in the targeted cancer cell. The α-enolase-specific targeting component can be a nucleic acid that hybridizes specifically to sequences encoding α-enolase or a portion of the α-enolase polypeptide. In other embodiments, the α-enolase-specific targeting component is selected from the group consisting of peptides, peptidomimetic compounds, small molecules specifically designed to bind α-enolase, and inhibitors of α-enolase. The aforementioned compounds are not intended to limit the range of compounds that can serve as the α-enolase-specific targeting component, but are merely illustrative examples.

The α-enolase binding component can also be plasminogen or an α-enolase specific binding fragment thereof. Moreover, α-enolase binding components can be composed of inhibitors of α-enolase. Alternatively, the α-enolase targeting agent is an interfering RNA (RNAi) that specifically hybridizes to a segment or region of the α-enolase nucleic acids expressed in the cancer cells. Ribonucleic acids used in RNAi to hybridize to target sequences can be of lengths between 10 to 20 bases, between 9 to 21 bases, between 7 to 23 bases, between 5 to 25 bases, between 25 to 35 bases, between 27 to 33 bases, and between 35 to 40 bases.

Following or at the time of treatment of a patient with α-enolase targeted therapy, chemotherapeutic treatment is administered. Non-limiting examples of useful chemotherapeutic drugs for treating a patient include Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, and Vinorelbine. These drugs are commercially obtainable, e.g., from ScienceLab.com, Inc. (Kingwood, Tex.). Physician administered treatment with these chemotherapeutic drugs is well known in the art (see, e.g., Capers et al., (1993) Hosp. Pharm. 28(3):206-10).

Aspects of the invention additionally provide kits for detecting neoplasms in a patient or cell sample and/or detecting chemotherapeutic drug resistance in neoplastic cells. The kits include probes for the detection of α-enolase and probes for the detection of vimentin, HSC70, and nucleophosmin. A patient that potentially has a tumor or the potential to develop a tumor can be tested for the presence of a tumor or tumor potential by determining the level of expression of α-enolase in a cell sample derived from the patient. In some embodiments, the kit provides probes that can be introduced into the patient by any acceptable means including, but not limited to, injection and surgical implantation.

The kits are also used to detect the presence or development of chemotherapeutic drug resistance in a neoplasm. During the course of patient chemotherapeutic treatment, monitoring of α-enolase, and other MDR-associated markers described herein, provides valuable information regarding the efficacy of the treatment and for avoiding the development of chemotherapeutic drug resistance. As shown above for neoplasm detection, the probes are provided that can be introduced into a patient by any acceptable means.

For both cancer detection and diagnosis of chemotherapeutic drug resistance, the kit can comprise a labeled compound or agent capable of detecting α-enolase protein in a biological sample; as well as means for determining the amount of α-enolase in the sample; and means for comparing the amount of α-enolase in the sample with a standard (e.g., normal cells of the same tissue type, normal non-neoplastic cells or non-MDR neoplastic cells). The compound or agent can be packaged in a suitable container. The kit can further comprise instructions for using the kit to detect α-enolase protein, as well as other MDR-associated markers. Such a kit can comprise, e.g., one or more antibodies that bind specifically to at least a portion of an α-enolase protein on a neoplastic cell.

The kit can also contain nucleic acids that are capable of detecting α-enolase expression in a cell sample. Non-limiting examples of nucleic acids include single-stranded RNA, double-stranded RNA, double-stranded DNA, single-stranded DNA, and RNA-DNA hybrids. Furthermore, nucleic acids can be labeled as described herein.

The kit contains a second probe for detection of MDR protein expression, which indicate the presence of chemotherapeutic drug resistance. These probes advantageously allow health professionals to obtain an additional data point to determine whether chemotherapeutic drug resistance exists. The probes can be labeled antibodies or fragments thereof capable of binding at least a portion of the chemotherapeutic drug resistance markers. Additionally, the probes can be nucleic acids capable of hybridizing to a region of a chemotherapeutic drug resistance marker. Vimentin, nucleophosmin, and HSC70 can be used as MDR proteins. However, other MDR proteins are known in the art and can be used in the present aspect of the invention (see, e.g., Ojima et al. (2005) J. Med. Chem. 48(6):2218-28; Matsumoto et al. (2005) J. Med. Invest. 52(1-2):41-8).

In another aspect of the invention, kits are provided that allow for the detection of metastatic potential in neoplastic cell samples. The kits provide a probe for the detection of cell surface α-enolase protein expression, which can be advantageous for the treatment of a neoplasm in a patient. During the course of patient chemotherapeutic treatment, monitoring of cell surface α-enolase, and other MDR proteins described herein, provides valuable information regarding the efficacy of the treatment and for avoiding the development of chemotherapeutic drug resistance. For example, the kit can comprise a labeled compound or agent capable of detecting cell surface α-enolase protein in a biological sample; as well as means for determining the amount of cell surface α-enolase in the sample; and means for comparing the amount of α-enolase in the sample with a standard (e.g., normal non-neoplastic cells or non-MDR neoplastic cells). The compound or agent can be packaged in a suitable container. The kit can further comprise instructions for using the kit to detect cell surface α-enolase protein, as well as other MDR-associated markers. Such a kit can comprise, e.g., one or more antibodies capable of binding specifically to at least a portion of a cell surface α-enolase protein.

Other aspects of the invention provide a vaccine for the treatment and prevention of metastatic disease in a patient. As used herein, the term “vaccine” means any formulation introduced into the body for the specific purpose of generating a specific immune response in a patient. Vaccines have been used to treat disease conditions, typically occurring due to infection of particular viruses (see, e.g., Desombere et al. (2005) Clin. Exp. Immunol. 140(1):126-37). Recently, vaccines have been utilized to treat various forms of cancer (see, e.g., Nestle et al. (2005) Curr. Opin. Immunol. 17(2):163-9). Accordingly, vaccine formulations against α-enolase can be used to treat metastatic disease arising from cancers such as a melanoma cell, a breast cancer cell, an ovarian cancer cell, a lung cancer cell, a lymphoma cell, a sarcoma cell, a leukemia cell, a retinoblastoma cell, a hepatoma cell, a myeloma cell, a glioma cell, a mesothelioma cell, a adenocarcinoma cell, and a carcinoma cell.

1.2 Targeting Agents

The present invention utilizes an α-enolase targeting agent for use in preventing or treating neoplasms and/or chemotherapeutic drug-resistant neoplasms. In some embodiments, targeting agents are used to increase the rate of apoptosis in a target cell population. In other embodiments, α-enolase targeting agents are used to inhibit angiogenic potential in neoplastic cells, which is necessary for the tumors to obtain nutrients. In some cases, α-enolase targeting agents prevent metastatic potential of neoplastic cells. It should be noted that α-enolase targeting agents treat chemotherapeutic drug resistant cells as well as neoplastic cells. In some instances, targeting agents can be in the form of proteins (hereinafter termed “protein-targeting agents”). As used herein, the term “protein-targeting agents” means a protein molecule or fragment thereof that can interact, bind, or associate with a molecule in a sample. A protein-targeting agent can be a protein or polypeptide capable of binding a biological macromolecule such as a protein, nucleic acid, simple carbohydrate, complex carbohydrate, fatty acid, lipoprotein, and/or triacylglyceride. Exemplary protein targeting agents include natural ligands of a receptor, hormones, antibodies, and portions thereof. The techniques associated with the binding of ligands and hormones to proteins as targeting agents have been demonstrated previously (see, e.g., Cutting et al., (2004) J. Biomol. NMR. 30(2):205-10).

In particular embodiments, the invention provides protein-targeting agents that are composed of antibodies or fragments of antibodies. These embodiments are described in detail below. The invention allows for antibodies to be immobilized on a solid support such as an antibody array where the support can be a bead or flat surface similar to a slide. An antibody microarray can determine the MDR protein expression of a chemotherapeutic drug-resistant cancer cell sample and the MDR protein expression of a multi-drug-sensitive control cell of the same tissue type. Alternatively, antibodies can be free in solution. Antibodies can also be conjugated to a non-limiting material such as magnetic compounds, paramagnetic compounds, proteins, nucleic acids, antibody fragments, or combinations thereof. In some embodiments, antibodies are used to inhibit α-enolase to decrease the “effective activity” of the enzyme in a targeted cell, thereby increasing the chemosensitivity of the cell to chemotherapeutic treatments (see Lopez-Alemany et al. (2003) Am. J. Hematol. 72(4): 234-42).

Protein targeting agents, including antibodies, can be detectably labeled. As used herein, “detectably labeled” means that a targeting agent is operably linked to a moiety that is detectable. By “operably linked” is meant that the moiety is attached to the targeting agent by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

Useful labels can be, without limitation, fluorophores (e.g., fluorescein (FITC), phycoerythrin, rhodamine), chemical dyes, or compounds that are radioactive, chemoluminescent, magnetic, paramagnetic, promagnetic, or enzymes that yield a product that may be colored, chemoluminescent, or magnetic. The signal is detectable by any suitable means, including spectroscopic, photochemical, biochemical, immunochemical, electrical, optical or chemical means. In certain cases, the signal is detectable by two or more means.

Labeled protein targeting agents allow detection of the level of expression of α-enolase in a cancer cell sample. For example, protein-targeting agents can be labeled for detection using chemiluminescent tags affixed to amino acid side chains. Useful tags include, but are not limited to, biotin, fluorescent dyes such as Cy5 and Cy3, and radiolabels (see, e.g., Barry and Soloviev (2000) Proteomics. 4(12): 3717-3726). Tags can be affixed to the amino terminal portion of a protein or the carboxyl terminal portion of a protein (see, e.g., Mattison and Kenney, (2002) J. Biol. Chem., 277(13): 11143-11148; Berne et al., (1990) J. Biol. Chem. 265(32): 19551-9). Indirect detection means can also be used to identify the cell markers. Exemplary but non-limiting means include detection of a primary antibody using a fluorescently labeled secondary antibody, or an antibody tagged with biotin such that it can be detected with fluorescently labeled streptavidin.

As used herein, a “nucleic acid targeting agent” is defined as a nucleic acid capable of binding to a target nucleic acid of complementary sequence through one or more types of chemical bonds, usually through complementary base pairing, usually through hydrogen bond formation. Nucleic acid targeting agents include, but are not limited to, single-stranded RNA, double-stranded RNA, single-stranded DNA, double-stranded DNA, cDNA, cRNA, DNA-RNA hybrids, and aptamers. Single-stranded RNAs also include siRNA and antisense RNA. A nucleic acid targeting agent may include natural (i.e. A, G, U, C, or T) or modified (7-deazaguanosine, inosine, etc.) bases. In addition, the bases in targeting agents may be joined by a linkage other than a phosphodiester bond, so long as it does not interfere with hybridization. Thus, nucleic acid targeting agents may be peptide nucleic acids in which the constituent bases are joined by peptide bonds rather than phosphodiester linkages. The nucleic acid targeting agents may be prepared by converting the RNA to cDNA using known methods (see, e.g., Ausubel et. al., Current Protocols in Molecular Biology Wiley 1999). The targeting agents can also be cRNA (see, e.g., Park et. al., (2004) Biochem. Biophys. Res. Commun. 325(4):1346-52).

Nucleic acid targeting agents can be produced from synthetic methods such as phosphoramidite methods, H-phosphonate methodology, and phosphite trimester methods. Nucleic acid targeting agents can also be produced by PCR methods. Such methods produce cDNA and cRNA sequences complementary to the mRNA. Such nucleic acid targeting agents can be detectably labeled, with, e.g., fluorophores (e.g., fluorescein (FITC), phycoerythrin, rhodamine), chemical dyes, or compounds that are radioactive, chemoluminescent, magnetic, paramagnetic, promagnetic, or enzymes that yield a product that may be colored, chemoluminescent, or magnetic. The signal is detectable by any suitable means, including spectroscopic, photochemical, biochemical, immunochemical, electrical, optical or chemical means. In certain cases, the signal is detectable by two or more means. In certain embodiments, nucleic acid labels include fluorescent dyes, radiolabels, and chemiluminescent labels, which are examples that are not intended to limit the scope of the invention (see, e.g., Yu, et al., (1994) Nucleic Acids Res. 22(16): 3226-3232; Zhu, et al., (1994) Nucleic Acids Res. 22(16): 3418-3422).

Nucleic acid targeting agents can be detectably labeled using fluorescent labels. Non-limiting examples of fluorescent labels include 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4α-diaza-s-indacene dyes and fluorescent proteins (e.g., green fluorescent protein, phycobiliprotein). These labels can be commercially obtained, e.g., from PerkinElmer Corp. (Boston, Mass.).

Other useful dyes are chemiluminescent dyes and can include, without limitation, biotin conjugated DNA nucleotides and biotin conjugated RNA nucleotides. Labeling of nucleic acid targeting agents can be accomplished by any means known in the art. (see, e.g., CyScribe™ First Strand cDNA Labeling Kit (#RPN6200, Amersham Biosciences, Piscataway, N.J.). The label can be added to the target nucleic acid(s) prior to, or after the hybridization. So called “direct labels” are detectable labels that are directly attached to, or incorporated into, the target nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore binds the biotin bearing hybrid duplexes providing a label that is easily detected. (see, e.g., Laboratory Techniques in Biochemistry and Molecular Biology, Vol. 24: Hybridization With Nucleic Acid Targeting Agents, P. Tijssen, ed. Elsevier, N.Y., (1993)).

The targeting agents of the present invention can also include inhibitors of α-enolase. Non-limiting examples of such inhibitors include phosphonoacetohydroxamate, (3-hydroxy-2-nitropropyl)phosphonate, (nitroethyl)phosphonate, and (phosphonoethyl)nitrolate. Labeled inhibitors have been shown to be acceptable targeting agents for binding and identifying compounds in complex solutions (see, e.g., Singh and Wyeth (1991) Int. J. Rad. Appl. Instrum. [A]. 42(3):251-9). Inhibitors can be labeled with detectable labels such as radiolabels, fluorescent labels, and chemiluminescent labels so long as the detectable label does not interfere with the binding or association of the inhibitor with its target compound (see, e.g., Singh and Wyeth (1991) Int. J. Rad. Appl. Instrum. [A]. 42(3):251-9).

Furthermore, plasminogen can be used as an α-enolase targeting agent for both membrane associated α-enolase and α-enolase isolated from the cytoplasmic fraction of the cell. Plasminogen has been identified previously as a ligand for membrane-associated α-enolase, which acts as a receptor when it is localized to the cell membrane (see, e.g., Pancholi (2001) Cell Mol. Life Sci. 58(7): 902-20). Fragments of plasminogen that show specific binding to α-enolase can also be used to bind to the protein. In addition, plasminogen or fragments thereof can be conjugated to relatively inert supports including, but not limited to, sepharose, cellulose, polystyrene, polyethylene glycol, and Sephadex®.

Plasminogen or fragments thereof can be attached to detectable labels. Non-limiting examples of detectable labels include fluorescent dyes such as Cy3/Cy5 protein dyes, radiolabels, biotin hydrazides, and biotin hydroxylamine (see, e.g., Jona et. al., (2003) Curr. Opin. Mol. Therap. 5(3): 271-277; Bacarese-Hamilton et. al., (2003) Curr. Opin. Mol. Therap. 5(3): 278-284). Alternatively, the detectable label can be attached to a compound that binds to the plasminogen or fragment thereof. In certain instances, the detectable label is attached to an antibody or antibody fragment specific for an epitope on plasminogen or a fragment thereof. Plasminogen antibodies can be obtained commercially (e.g., Research Diagnostics, Inc., Flanders, N.J.). It should be noted that “indirect labels” can be joined to a binding moiety other than an antibody that is specific for α-enolase. Examples of other binding moieties, which are not intended to be limiting, include nucleic acids, proteins, peptides, and peptidomimetic compounds.

In addition, aptamers can be α-enolase targeting agents. The term “aptamer,” used herein interchangeably with the term “nucleic acid ligand,” means a nucleic acid that, through its ability to adopt a specific three-dimensional conformation, binds to and has an antagonizing (i.e., inhibitory) effect on a target. The target of the present invention is α-enolase, and hence the term α-enolase aptamer or nucleic acid ligand is used. Inhibition of the target by the aptamer may occur by binding of the target, by catalytically altering the target, by reacting with the target in a way which modifies/alters the target or the functional activity of the target, by covalently attaching to the target as in a suicide inhibitor, by facilitating the reaction between the target and another molecule. Aptamers may be comprised of multiple ribonucleotide units, deoxyribonucleotide units, or a mixture of both types of nucleotide residues. Aptamers may further comprise one or more modified bases, sugars or phosphate backbone units as described above.

Aptamers can be made by any known method of producing oligomers or oligonucleotides. Many synthesis methods are known in the art. For example, 2′-O-allyl modified oligomers that contain residual purine ribonucleotides, and bearing a suitable 3′-terminus such as an inverted thymidine residue (Ortigao et al., (1992) Antisense Res. Devel. 2:129-146) or two phosphorothioate linkages at the 3′-terminus to prevent eventual degradation by 3′-exonucleases, can be synthesized by solid phase beta-cyanoethyl phosphoramidite chemistry (Sinha et al., Nucleic Acids Res., 12:4539-4557 (1984)) on any commercially available DNA/RNA synthesizer. One method is the 2′-O-tert-butyldimethylsilyl (TBDMS) protection strategy for the ribonucleotides (Usman et al., (1987) J. Am. Chem. Soc., 109: 7845-7854), and all the required 3′-O-phosphoramidites are commercially available. In addition, aminomethylpolystyrene may be used as the support material due to its advantageous properties (McCollum and Andrus (1991) Tetrahedron Lett., 32:4069-4072). Fluorescein can be added to the 5′-end of a substrate RNA during the synthesis by using commercially available fluorescein phosphoramidites. In general, an aptamer oligomer can be synthesized using a standard RNA cycle. Upon completion of the assembly, all base labile protecting groups are removed by an eight hour treatment at 55° C. with concentrated aqueous ammonia/ethanol (3:1 v/v) in a sealed vial. The ethanol suppresses premature removal of the 2′-O-TBDMS groups that would otherwise lead to appreciable strand cleavage at the resulting ribonucleotide positions under the basic conditions of the deprotection (Usman et al., (1987) J. Am. Chem. Soc., 109: 7845-7854). After lyophilization, the TBDMS protected oligomer is treated with a mixture of triethylamine trihydrofluoride/triethylamine/N-methylpyrrolidinone for 2 hours at 60° C. to afford fast and efficient removal of the silyl protecting groups under neutral conditions (see Wincott et al., (1995) Nucleic Acids Res., 23:2677-2684). The fully deprotected oligomer can then be precipitated with butanol according to the procedure of Cathala and Brunel ((1990) Nucleic Acids Res., 18:201). Purification can be performed either by denaturing polyacrylamide gel electrophoresis or by a combination of ion-exchange HPLC (Sproat et al., (1995) Nucleosides and Nucleotides, 14:255-273) and reversed phase HPLC. For use in cells, synthesized oligomers are converted to their sodium salts by precipitation with sodium perchlorate in acetone. Traces of residual salts may then be removed using small disposable gel filtration columns that are commercially available. As a final step the authenticity of the isolated oligomers may be checked by matrix assisted laser desorption mass spectrometry (Pieles et al., (1993) Nucleic Acids Res., 21:3191-3196) and by nucleoside base composition analysis.

The disclosed aptamers can also be produced through enzymatic methods, when the nucleotide subunits are available for enzymatic manipulation. For example, the RNA molecules can be made through in vitro RNA polymerase T7 reactions. They can also be made by strains of bacteria or cell lines expressing T7, and then subsequently isolated from these cells. As discussed below, the disclosed aptamers can also be expressed in cells directly using vectors and promoters.

The aptamers, like other nucleic acid molecules of the invention, may further contain chemically modified nucleotides. One issue to be addressed in the diagnostic or therapeutic use of nucleic acids is the potential rapid degradation of oligonucleotides in their phosphodiester form in body fluids by intracellular and extracellular enzymes such as endonucleases and exonucleases before the desired effect is manifest. Certain chemical modifications of the nucleic acid ligand can be made to increase the in vivo stability of the nucleic acid ligand or to enhance or to mediate the delivery of the nucleic acid ligand (see, e.g., U.S. Pat. No. 5,660,985).

The stability of the aptamer can be greatly increased by the introduction of such modifications and as well as by modifications and substitutions along the phosphate backbone of the RNA. In addition, a variety of modifications can be made on the nucleobases themselves, which both inhibit degradation and which can increase desired nucleotide interactions or decrease undesired nucleotide interactions. Accordingly, once the sequence of an aptamer is known, modifications or substitutions can be made by the synthetic procedures described below or by procedures known to those of skill in the art.

Other modifications include the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). The aptamers may also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer may further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer may still further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil.

Examples of other modified base variants known in the art include, without limitation, e.g., 4-acetylcytidine, 5-(carboxyhydroxylmethyl) uridine, 2′-methoxycytidine, 5-carboxymethylaminomethyl-2-thioridine, 5-carboxymethylaminomethyluridine, dihydrouridine, 2′-O-methylpseudouridine, b-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N6-isopentenyladenosine, N-((9-b-D-ribofuranosyl-2-methylthiopurine-6-yl)carbamoyl)threonine, N-((9-b-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-b-D-ribofuranosylpurine-6-yl)carbamoyl)threonine, 2′-O-methyl-5-methyluridine, 2′-O-methyluridine, and wybutosine, 3-(3-amino-3-carboxypropyl)uridine.

Also included are the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941. Examples of modified nucleoside and nucleotide sugar backbone variants known in the art include, without limitation, those having, e.g., 2′ ribosyl substituents such as F, SH, SCH3, OCN, Cl, Br, CN, CF3, OCF3, SOCH3, SO2, CH3, ONO2, NO2, N3, NH2, OCH2CH2OCH3, O(CH2)2ON(CH3)2, OCH2OCH2N(CH3)2, O(C1-10 alkyl), O(C2-10 alkenyl), O(C2-10 alkynyl), S(C1-10 alkyl), S(C2-10 alkenyl), S(C2-10 alkynyl), NH(C1-10 alkyl), NH(C2-10 alkenyl), NH(C2-10 alkynyl), and O-alkyl-O-alkyl. Desirable 2′ ribosyl substituents include 2′-methoxy (2′-OCH3), 2′-aminopropoxy (2′ OCH2CH2CH2NH2), 2′-O-allyl (2′-CH2—CH═CH2), 2′-O-allyl (2′-O—CH2—CH═CH2), 2′-amino (2′-NH2), and 2′-fluoro (2′-F). The 2′-substituent may be in the arabino (up) position or ribo (down) position.

Aptamers may be made up of nucleotides and/or nucleotide analogs such as described above, or a combination of both, or are oligonucleotide analogs. Aptamers may contain nucleotide analogs at positions, which do not affect the function of the oligomer to bind α-enolase.

There are several techniques that can be adapted for refinement or strengthening of the nucleic acid ligands binding to a particular target molecule or the selection of additional aptamers. One technique, generally referred to as “in vitro genetics” (see Szostak (1992) TIBS, 19:89), involves isolation of aptamer antagonists by selection from a pool of random sequences. The pool of nucleic acid molecules from which the disclosed aptamers may be isolated may include invariant sequences flanking a variable sequence of approximately twenty to forty nucleotides. This method has been termed Selective Evolution of Ligands by Exponential Enrichment (SELEX). Compositions and methods for generating aptamer antagonists of the invention by SELEX and related methods are known in the art and taught in, for example, U.S. Pat. Nos. 5,475,096 and 5,270,163. The SELEX process in general is further described in, e.g., U.S. Pat. Nos. 5,668,264, 5,696,249, 5,670,637, 5,674,685, 5,723,594, 5,756,291, 5,811,533, 5,817,785, 5,958,691, 6,011,020, 6,051,698, 6,147,204, 6,168,778, 6,207,816, 6,229,002, 6,426,335, and 6,582,918.

Other modifications useful for producing aptamers of the invention are known to one of ordinary skill in the art. Such modifications may be made post-SELEX process (modification of previously identified unmodified ligands) or by incorporation into the SELEX process. It has been observed that aptamers, or nucleic acid ligands, in general, are most stable, and therefore efficacious when 5′-capped and 3′-capped in a manner which decreases susceptibility to exonucleases and increases overall stability.

α-enolase targeting agents are specifically targeted to a neoplasm to prevent detection of α-enolase activity in normal cells of the patient. Targeting mechanisms include non-limiting techniques such as conjugating the α-enolase targeting agent to an agent that binds preferentially to a cancer cell marker (hereinafter termed “cancer cell targeting agent”). Cancer cell targeting agents include, but are not limited to, antibodies or binding fragments thereof, nucleic acids, peptides, small molecules, and peptidomimetic compounds. Cancer cell targeting agents can be conjugated directly to the α-enolase targeting agent, for example, through covalent bonding to, e.g., carboxyl, phosphoryl, sulfhydryl, carbonyl, and hydroxyl groups using chemical techniques known in the art. Alternatively, cancer cell targeting agents and α-enolase targeting agents can be conjugated to functionalized chemical groups on non-limiting examples of inert supports such as polyethylene glycol, glass, synthetic polymers such as polyacrylamide, polystyrene, polypropylene, polyethylene, or natural polymers such as cellulose, Sepharose, or agarose, or conjugates with enzymes. Chemical conjugation techniques are well known in the art. Non-limiting examples of cancer cell markers that can be used for targeting of α-enolase targeting agent include Pgp-1, MRP1, BIP, BRCP, HSC70, nucleophosmin, vimentin, and HSP90.

Alternatively, the α-enolase targeting agent can be targeted to a neoplasm through a variety of invasive procedures. In the context of the present embodiment, such procedures include catheterization through an artery of a patient and depositing the α-enolase targeting agent within the tumor site. A surgeon can also apply the α-enolase targeting agent to the neoplasm by making an incision into the patient at a site that allows access to the tumor for placement of the α-enolase targeting agent into, onto, or in close proximity to, the tumor. In some instances, a subject can also be intubated with subsequent introduction of the α-enolase targeting agent into the tumor site through the tube. In other embodiments, the α-enolase targeting agent can be administered to a patient orally, subcutaneously, intramuscularly, intravenously, or interperitoneally.

The α-enolase targeting agent can be incorporated into a liposome before it is used. The term “liposome”, as used herein, refers to an artificial phospholipid bilayer vesicle. The liposome formulation can be used to facilitate lipid bilayer fusion with a target cell, thereby allowing the contents of the liposome or proteins associated with its surface to be brought into contact with the neoplastic cell. Liposomes can have antibodies associated with their bilayers that allow binding to targets on the neoplastic cell surface (hereinafter termed “immunoliposome”). Antibodies for these cell markers can be obtained commercially (e.g., Research Diagnostics, Inc., Flanders, N.J.; and Abcam, Inc., Cambridge, Mass.). Non-limiting examples of neoplastic cell targets to which such antibodies are specifically directed include Pgp-1, MRP1, BIP, BRCP, HSC70, nucleophosmin, vimentin, and HSP90.

1.3 Antibodies for Detection of α-Enolase

Aspects of the present invention utilize antibodies directed against α-enolase for use in diagnosis, detection, and prevention of chemotherapeutic drug-resistant cancer cells. Moreover, aspects of the present invention utilize treating/preventing cancer in a patient. Furthermore, the present invention utilizes antibodies against α-enolase in methods for diagnosis, detection, and prevention of metastatic cancer cells. Anti-α-enolase antibodies, both monoclonal and polyclonal, for use in the invention are available from several commercial sources (e.g., Santa Cruz Biotechnology, Santa Cruz, Calif.; and Biogenesis, Inc., Kingston, N.H.). α-enolase antibodies can be administered to a patient orally, subcutaneously, intramuscularly, intravenously, or interperitoneally.

Aspects of the invention also utilize polyclonal antibodies for the detection α-enolase and/or the treatment/prevention of cancer in a patient. As used herein, the term “polyclonal antibodies” means a population of antibodies that can bind to multiple epitopes on an antigenic molecule. A polyclonal antibody is specific to a particular epitope on an antigen, while the entire pool of polyclonal antibodies can recognize different epitopes. In addition, polyclonal antibodies developed against the same antigen can recognize the same epitope on an antigen, but with varying degrees of specificity. Polyclonal antibodies can be isolated from multiple organisms including, but not limited to, rabbit, goat, horse, mouse, rat, and primates. Polyclonal antibodies can also be purified from crude serums using techniques known in the art (see, e.g., Ausubel, et al., Current Protocols in Molecular Biology Vol. 1, pp. 4.2.1-4.2.9, John Wiley & Sons, Inc., 1996).

The term “monoclonal antibody”, as used herein, refers to an antibody obtained from a population of substantially homogenous antibodies, i.e., the individual antibodies comprising the population are identical except for possible naturally occurring mutations that may be present in minor amounts. Monoclonal antibodies are highly specific, being directed against a single antigenic site. By their nature, monoclonal antibody preparations are directed to a single specific determinant on the target. Novel monoclonal antibodies or fragments thereof mean in principle all immunoglobulin classes such as IgM, IgG, IgD, IgE, IgA, or their subclasses or mixtures thereof. Non-limiting examples of subclasses include the IgG subclasses IgG1, IgG2, IgG3, IgG2a, IgG2b, IgG3, or IgGM. The IgG subtypes IgG1/κ and IgG2b/κ are also included within the scope of the present invention.

The monoclonal antibodies herein include hybrid and recombinant antibodies produced by splicing a variable (including hypervariable) domain of an anti-α-enolase antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567; Mage and Lamoyi, in Monoclonal Antibody Production Techniques and Applications, (Marcel Dekker, Inc., New York 1987, pp. 79-97). Thus, the modified “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method (see, e.g., Kohler and Milstein (1975) Nature 256:495) or can be made by recombinant DNA methods (U.S. Pat. No. 4,816,567). The monoclonal antibodies can also be isolated from phage libraries generated using the techniques described in the art (see, e.g., McCafferty et al. (1990) Nature 348:552-554).

Alternative methods for producing antibodies can be used to obtain high affinity antibodies. Antibodies for α-enolase can be obtained from human sources such as serum. Additionally, monoclonal antibodies can be obtained from mouse-human heteromyeloma cell lines by techniques known in the art (see, e.g., Kozbor (1984) J. Immunol. 133, 3001; Boerner et al., (1991) J. Immunol. 147:86-95). Methods for the generation of human monoclonal antibodies using phage display, transgenic mouse technologies, and in vitro display technologies are known in the art and have been described previously (see, e.g., Osbourn et al. (2003) Drug Discov. Today 8: 845-51; Maynard and Georgiou (2000) Ann. Rev. Biomed. Eng. 2: 339-76; U.S. Pat. Nos. 4,833,077; 5,811,524; 5,958,765; 6,413,771; and 6,537,809).

1.4 RNA Interference

Aspects of the present invention further allow for the treatment of a patient with a neoplasm or, in some embodiments, for the treatment of chemotherapeutic drug-resistant neoplasms using RNA interference (“RNAi”). As used herein, the term “RNA interference” refers to the blocking or preventing of cellular production of a particular protein by stopping the mechanisms of translation using small RNAs that hybridize to complementary sequences in a target mRNA. Anti-sense RNA strategies utilize the single-stranded nature of mRNA in a cell to block or interfere with translation of the mRNA into a protein. Antisense technology has been the most commonly described approach in protocols to achieve gene-specific interference. For antisense strategies, stoichiometric amounts of single-stranded nucleic acid complementary to the messenger RNA for the gene of interest are introduced into the cell.

The RNA may comprise one or more strands of polymerized ribonucleotide. It may include modifications to either the phosphate-sugar backbone or the nucleoside. For example, the phosphodiester linkages of natural RNA may be modified to include at least one of a nitrogen or sulfur heteroatom. For example, structural groups may be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid may be substituted or modified, for example with methyl phosphonates or O-methyl phosphates. Bases and sugars can also be modified, as is known in the art. RNA can also be modified to include “peptide nucleic acids” in which native or modified nucleic acid bases are attached to a polyamide backbone. Modifications in RNA structure may be tailored to allow specific genetic inhibition while avoiding a general panic response in some organisms, which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

Methods of using siRNA to inhibit gene expression are well known in the art (see e.g., U.S. Pat. No. 6,506,559). Typically, complementary RNA sequences that can hybridize to a specific region of the target RNA are introduced into the cell. RNA annealing to the target transcripts allows the internal machinery of the cell to cut the dsRNA sequences into short segments. Such mechanisms have been utilized in in vitro and in vivo studies of human genes (see, e.g., Mizutani et al. (2002) J. Biol. Chem. 277(18):15859-64; Wang et al. (2005) Breast Cancer Res. 7(2):R220-8). In particular, the c-myc gene was inhibited in MCF7 breast cancer cell lines using the RNA interference technique (see Wang et al. (2005) Breast Cancer Res. 7(2):R220-8).

Interfering RNAs can be obtained by any means known in the art. For example, interfering RNA can be synthetically produced using the Expedite™ Nucleic Acid Synthesizer (Applied Biosystems, Foster City, Calif.) or other similar devices (see, e.g., Applied Biosystems, Foster City, Calif.). Synthetic oligonucleotides also can be produced using methods well known in the art such as phosphoramidite methods (see, e.g., Pan et. al., (2004) Biol. Proc. Online. 6:257-262), H-phosphonate methodology (see, e.g., Agrawal et. al., (1987) Tetrahedron Lett. 28(31): 3539-3542) and phosphite trimester methods (Finnan et al. (1980) Nucleic Acids Symp. Ser. (7): 133-45).

1.5 Diagnostic Methods for Detection of α-Enolase

Aspects of the invention allow the identification of patients having such neoplastic cells, which may be chemotherapeutic drug resistant or express the characteristics of MDR neoplastic cells. Other aspects of the invention allow for the detection of neoplasms in a patient. Such patients are potentially harboring neoplastic cells, which may be chemotherapeutic drug resistant, and are therefore candidates for diagnostics directed to identifying the potential for a neoplasm. In some instances, the patient is a member of a high risk group for developing cancer. For example, where the patient identified as potentially having such cells is a patient in remission of cancer or is being treated for cancer (e.g., a patient suffering from breast cancer, ovarian cancer, lung cancer, prostate cancer, leukemia, etc.), the invention allows identification of these patients prior to resurgence and/or progression of their cancer, as well as allows the monitoring of these patients during treatment with a drug, such that the treatment regimen can be altered. In addition, some patients carry certain mutations in genes that predispose the patients to cancer development. For instance, female carriers of the BRCA 1 and BRCA 2 alleles are predisposed to the development of breast cancer. Therefore, the present invention allows for diagnostic assays that can be utilized to determine the presence of cancer in patients that are potential carriers of neoplastic cells.

The diagnostic applications of the invention include probes and other detectable agents that are joined to a α-enolase targeting agent, such as an anti-α-enolase antibody. Conjugation of such agents to the targeting agent can be accomplished by, e.g., covalent bonding to non-limiting active groups such as carbonyls, carboxyls, amines, amides, hydroxyls, and sulfhydryls. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y. 1999).

In accordance with the invention, a detectably labeled targeting agent of the invention includes a targeting agent that is conjugated to a detectable moiety. Another detectably labeled targeting agent of the invention is a fusion protein, where one component is the targeting agent and the other component is a detectable label. Yet another non-limiting example of a detectably labeled targeting agent is a first fusion protein comprising a targeting agent and a first moiety with high affinity to a second moiety, and a second fusion protein comprising a second moiety and a detectable label. For example, a targeting agent that specifically binds to an α-enolase protein may be operably linked to a streptavidin moiety. A second fusion protein comprising a biotin moiety operably linked to a fluorescein moiety may be added to the targeting agent-streptavidin fusion protein, where the combination of the second fusion protein to the targeting agent-streptavidin fusion protein results in a detectably labeled targeting agent (i.e., a targeting agent operably linked to a detectable label). Detectable labels have been described above.

Measuring the level of expression of a α-enolase protein on the surface of the neoplastic cell includes contacting the intact neoplastic cell with a detectable targeting agent that specifically binds to a α-enolase protein. For example, where the detectable targeting agent is detectably labeled by being operably linked to a fluorophore, cells staining with the fluorophore (i.e., those that are specifically bound by the targeting agent) can be identified by fluorescent activated cell sorter analysis, or by routine fluorescent microscopy of clinical specimens prepared on slides.

Useful detectable targeting agents are labeled antibodies, and derivatives and analogs thereof, which specifically bind to α-enolase polypeptide (see Section 1.3). These antibodies can be used for diagnostic purposes to detect, diagnose, or monitor diseases and/or disorders associated with the aberrant expression of α-enolase. The invention provides for the detection of aberrant expression of α-enolase (a) assaying the expression of the polypeptide of interest in cells or cell surface membrane fractions of an individual using one or more antibodies specific to α-enolase and (b) comparing the level of gene expression with a standard gene expression level, whereby an increase or decrease in the assayed α-enolase expression level compared to the standard expression level is indicative of aberrant expression. For example, where chemotherapeutic drug resistance in a neoplastic cell is to be detected, the standard expression level to which comparison should be made is a non-chemotherapeutic drug-resistant neoplastic cell of the same or similar origin or cell type. Similarly, where neoplasia in a test cell is to be detected, the standard expression level to which comparison should be made is a non-neoplastic cell of the same or similar origin or cell type.

The presence of increased α-enolase expression in biopsied tissue or test cell from an individual can indicate a predisposition for the development of chemotherapeutic drug resistance, or can provide a means for detecting chemotherapeutic drug resistance prior to the appearance of actual clinical symptoms. A more definitive diagnosis of this type allows health professionals to employ preventative measures or aggressive treatment earlier, thereby preventing the development or further progression of the cancer. In addition, the presence of increased α-enolase expression on the surface of biopsied cells from neoplastic tissues can indicate a predisposition for the development of metastatic disease in the neoplasm prior to actual manifestation of metastasis. Information of this type allows for clinicians to tailor their treatment choices accordingly, potentially preventing development of neoplastic disease in additional tissues within the patient.

Antibodies directed to α-enolase are also useful to assay protein levels in a biological sample using classical immunohistological methods known to those of skill in the art (see, e.g., Jalkanen et al., (1985) J. Cell. Biol. 101:976-985; Jalkanen et al. (1987) J. Cell. Biol. 105:3087-3096). Other antibody-based methods useful for detecting α-enolase protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin.

1.6 Liposome

Another strategy that may be employed for delivery of α-enolase targeting agent is the use of immunoliposomes. Immunoliposomes incorporate antibodies against tumor-associated antigens into liposomes, which carry the therapeutic agent, such as the α-enolase targeting agent, or an enzyme that activates an otherwise inactive prodrug (see, e.g., Lasic et al. (1995) Science 267: 1275-76). Immunoliposomal drugs can be used to successfully target and enhance anti-cancer efficacy (see, e.g., Maruyama et al. (1990) J. Pharm. Sci. 74: 978-84); Maruyama et al. (1995) Biochim. Biophys. Acta 1234: 74-80; Otsubo et al. (1998) Antimicrob. Agents Chemother. 42: 40-44; Lopes de Menezes et al. (1998) Cancer Res. 58: 3320-30).

α-enolase targeting agents can be incorporated into the membrane of the liposome through mechanisms known in the art (see, e.g., Pakunlu et al. (2004) Cancer Res. 64(17): 6214-24; Shimizu et al. (2002) Biol. Pharm. Bull. 25(6): 783-6; Zheng and Tan (2004) World J. Gastroenterol. 10(17): 2563-6). In addition, α-enolase targeting agents can be associated with the outside of a liposome through covalent linkages to PEG polymers (see, e.g., Medina et al. (2004) Curr. Pharm. Des. 10(24): 2981-9). Furthermore, targeting agents can be incorporated into the hydrated inner compartment of the liposome (see, e.g., Medina et al (2004) Curr. Pharm. Des. 10(24): 2981-9). A combination of the above mentioned liposome delivery methods can be used in a therapeutic composition.

Alternatively, modified LDL may be used as tumor-specific ligands in targeting liposomal formulations containing α-enolase targeting agents. For example, folate-coupled liposomes can be used to target therapeutics to tumors, which overexpress the folate receptor (Lee and Low (1994) J. Biol. Chem. 269: 3198-204; Lee and Low (1995) Biochim. Biophys. Acta 1233: 134-44; Rui et al. (1998) J. Am. Chem. Soc. 120: 11213-18; Gabizon et al. (1999) Bioconj. Chem. 10: 289-98). Transferrin has been employed as a targeting ligand to direct liposomal drugs to various types of cancer cell in vivo (Ishida and Maruyama (1998) Nippon Rinsho 56: 657-62; Kirpotin et al. (1997) Biochem. 36: 66-75). PEG-immunoliposomes with anti-transferring antibodies coupled to the distal ends of the PEG preferentially associate with C6 glioma cells in vitro and significantly increased gliomal doxorubicin uptake after treatment with the tumor-specific long-circulating liposomes containing doxorubicin (Eavarone et al. (2000) J. Biomed. Mater. Res. 51: 10-14).

Methods of delivering chemotherapeutic drugs and siRNA in vivo are known in the art (see, e.g., Mewani et al. (2004) Int. J. Oncol. 24(5): 1181-8; Chien et al. (2005) Cancer Gene Ther. 12(3): 3221-8). Liposomes have also been used for the targeted delivery of chemotherapeutic drugs, toxins, and labels (see, e.g., Pakunlu et al. (2004) Cancer Res. 64(17): 6214-24; Shimizu et al. (2002) Biol. Pharm. Bull. 25(6): 783-6; Zheng and Tan (2004) World J. Gastroenterol. 10(17): 2563-6). Liposome formulations for the delivery of chemotherapeutics and siRNA can be obtained from commercial suppliers, e.g., Eurogentec, Ltd. (Southampton, Hampshire, UK). In addition, methods for producing liposome micelle/chemotherapeutic formulations are well known in the art. For example, therapeutic drug micelles can be formed by combining a therapeutic drug and a phosphatidyl glycerol lipid derivative (PGL derivative). Briefly, the therapeutic drug and PGL derivative are mixed in a range of 1:1 to 1:2.1 to form a therapeutic drug mixture. Alternatively, the range of therapeutic drug to PGL derivative is 1:1.2; or 1:1.4; or 1:1.5; or 1:1.6; or 1:1.8 or 1:1.9 or 1:2.0 or 1:2.1. The mixture is then combined with an effective amount of at least a 20% organic solvent such as an ethanol solution to form micelles containing the therapeutic drug. Methods for inclusion of an antibody or tumor targeting ligand into the micelle formulation to produce immunoliposomes are known in the art and described further below. For example, methods for preparation and use of immunoliposomes are described in U.S. Pat. Nos. 4,957,735, 5,248,590, 5,464,630, 5,527,528, 5,620,689, 5,618,916, 5,977,861, 6,004,534, 6,027,726, 6,056,973, 6,060,082, 6,316,024, 6,379,699, 6,387,397, 6,511,676 and 6,593,308.

As used herein, the term “phosphatidyl glycerol lipid derivative (PGL derivative)” is any lipid derivative having the ability to form micelles and have a net negatively charged head group. This includes but is not limited to dipalmitoyl phosphatidyl glycerol (DPPG), dimyristoyl phosphatidyl glycerol, and dicapryl phosphatidyl glycerol. In one aspect, phosphatidyl derivatives with a carbon chain of 10 to 28 carbons and having unsaturated side aliphatic side chain are within the scope of this invention. The complexing of a therapeutic drug with negatively-charged phosphatidyl glycerol lipids having variations in the molar ratio giving the particles a net positive (1:1) neutral (1:2) or slightly negative (1:2.1) charge will allow targeting of different tissues in the body after administration. However, complexing of a therapeutic drug with negatively charged PGL has been shown to enhance the solubility of the therapeutic drug in many instances, thus reducing the volume of the drug required for effective antineoplastic therapy. In addition, the complexing of a therapeutic drug and negatively charged PGL proceeds to very high encapsulation efficiency, thereby minimizing drug loss during the manufacturing process. These complexes are stable, do not form precipitates and retain therapeutic efficacy after storage at 4° C. for at least four months. In order to achieve maximum therapeutic efficacy by avoiding rapid clearance from the blood circulation by the reticuloendothelial system (RES), immunoliposomal drug formulations may incorporate components such as polyethylene glycol (PEG) (see, e.g., Klibanov et al. (1990) FEBS Lett. 268: 235-7; Mayuryama et al. (1992) Biochim. Biophys. Acta 1128: 44-49; Allen et al. (1991) Biochim. Biophys. Acta 1066: 29-36). Long-circulating immunoliposomes can be classified into two types: those with antibodies coupled to a lipid head growth (Maruyama et al. (1990) J. Pharm. Sci. 74: 978-84); and those with antibodies coupled to the distal end of PEG (Maruyama et al. (1997) Adv. Drug Del. Rev. 24: 235-42). In certain instances, it may be advantageous to place the tumor-specific antibodies at the distal end of the PEG polymer to obtain efficient target binding by avoiding steric hindrance from the PEG chains.

1.7 α-Enolase Vaccines

The invention includes known methods of preparing and using tumor antigen vaccines for use in treating neoplasms, and treating chemotherapeutic drug-resistance in neoplasms. The invention also includes methods of preparing and using tumor antigen vaccines for use in preventing cancers or for use in preventing cancers from becoming chemotherapeutic drug-resistant, and for use in treating metastatic cancers or for use in preventing cancers from developing metastatic potential. The vaccine can be made using an α-enolase polypeptide, or α-enolase polypeptide fragment, and at least one pharmaceutically acceptable carrier.

A method of treating or preventing metastatic neoplasms in a subject comprises administering an effective amount of an α-enolase vaccine. Vaccines can be made to prevent the development of metastatic and/or angiogenic neoplasms from cells including, but not limited to, melanoma cells, breast cancer cells, ovarian cancer cells, lung cancer cells, lymphoma cells, sarcoma cells, leukemia cells, retinoblastoma cells, hepatoma cells, myeloma cells, glioma cells, mesothelioma cells, adenocarcinoma cells, and carcinoma cells. In addition, these cells can be obtained from various tissues such as breast, skin, lymphatic, prostate, bone, blood, brain, liver, thymus, kidney, lung, and ovary.

For example, U.S. Pat. No. 6,562,347 which teaches the use of a fusion polypeptide including a chemokine and a tumor antigen which is administered as either a protein or nucleic acid vaccine to elicit an immune response effective in treating or preventing cancer. Chemokines are a group of usually small secreted proteins (7-15 kD) induced by inflammatory stimuli and are involved in orchestrating the selective migration, diapedesis and activation of blood-born leukocytes that mediate the inflammatory response (see Wallack (1993) Ann. NY Acad. Sci. 178). Chemokines mediate their function through interaction with specific cell surface receptor proteins. At least four chemokine subfamilies have been identified as defined by a cysteine signature motif, termed CC, CXC, C and CX3C, where C is a cysteine and X is any amino acid residue. Structural studies have revealed that at least both CXC and CC chemokines share very similar tertiary structure (monomer), but different quaternary structure (dimer). For the most part, conformational differences are localized to sections of loop or the N-terminus. In the instant invention, for example, a human α-enolase polypeptide sequence (such as that shown in Table I), or polypeptide fragment thereof, and a chemokine sequence are fused together and used in an immunizing vaccine. The chemokine portion of the fusion can be a human monocyte chemotactic protein-3, a human macrophage-derived chemokine or a human SDF-1 chemokine. The α-enolase portion of the fusion is a portion shown in routine screening to have a strong antigenic potential. Immunological compositions, including vaccines, and other pharmaceutical compositions containing the α-enolase protein, or portions thereof, are used within the scope of the present invention. One or more of the α-enolase proteins, or active or antigenic fragments thereof, or fusion proteins thereof can be formulated and packaged, alone or in combination with other antigens, using methods and materials known to those skilled in the art for vaccines. The immunological response may be used therapeutically or prophylactically and may provide antibody immunity or cellular immunity, such as that produced by T lymphocytes.

To enhance immunogenicity, the proteins may be conjugated to a carrier molecule. Suitable immunogenic carriers include proteins, polypeptides or peptides such as albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular mass of at least 1 kD, greater than 10 kD. Carrier molecules often contain a reactive group to facilitate covalent conjugation to the hapten. The carboxylic acid group or amine group of amino acids or the sugar groups of glycoproteins are often used in this manner. Carriers lacking such groups can often be reacted with an appropriate chemical to produce them. An immune response is produced when the immunogen is injected into animals such as mice, rabbits, rats, goats, sheep, guinea pigs, chickens, and other animals such as mice and rabbits. Alternatively, a multiple antigenic peptide comprising multiple copies of the protein or polypeptide, or an antigenically or immunologically equivalent polypeptide may be sufficiently antigenic to improve immunogenicity without the use of a carrier.

The α-enolase protein or portions thereof, such as consensus or variable sequence amino acid motifs, or combination of proteins may be administered with an adjuvant in an amount effective to enhance the immunogenic response against the conjugate. One adjuvant widely used in humans is alum (aluminum phosphate or aluminum hydroxide). Saponin and its purified component Quil A, Freund's complete adjuvant and other adjuvants used in research and veterinary applications are also available. Chemically defined preparations such as muramyl dipeptide, monophosphoryl lipid A, phospholipid conjugates, encapsulation of the conjugate within a proteoliposome, and encapsulation of the protein in lipid vesicles such as Novasome™ lipid vesicles (Micro Vescular Systems, Inc., Nashua, N. H.) have been described previously (Goodman-Snitkoff et al. (1991) J. Immunol. 147:410415; Miller et al. (1992) J. Exp. Med. 176:1739-1744).

The invention utilizes α-enolase polypeptide fragments, or subsequences of the intact α-enolase polypeptide shown in Table 1 (SEQ ID NO: 1). Such α-enolase polypeptide subsequences, or a corresponding nucleic acid sequence that encodes them in the case of DNA vaccines, are selected so as to be highly immunogenic. The principles of antigenicity for the purpose of producing anti-α-enolase vaccines apply also to the use of α-enolase polypeptide sequences for use as immunogens for generating anti-α-enolase polyclonal and monoclonal antibodies for use in the α-enolase-based diagnostics and therapeutics described herein.

Furthermore, a suitable adjuvant is typically combined with the immunogenic compound of a vaccine. As used herein, “adjuvant” or “suitable adjuvant” describes a substance capable of being combined with the α-enolase protein or polypeptide to enhance an immune response in a subject without deleterious effect on the subject. A suitable adjuvant can be, but is not limited to, for example, an immunostimulatory cytokine, SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion. The adjuvant, such as an immunostimulatory cytokine can be administered before the administration of the α-enolase protein or α-enolase-encoding nucleic acid, concurrent with the administration of the α-enolase protein or α-enolase-encoding nucleic acid or up to five days after the administration of the α-enolase protein or α-enolase-encoding nucleic acid to a subject. QS-21, similarly to alum, complete Freund's adjuvant, SAF, etc., can be administered within hours of administration of the fusion protein.

1.8 Therapies

The invention provides for treatment or prevention of neoplasms, tumors, or metastases, and particularly chemotherapeutic drug-resistant forms thereof by the administration of therapeutically or prophylactically effective amounts of anti-α-enolase antibodies or nucleic acid molecules encoding said antibodies. Moreover, the present invention provides α-enolase therapies directed to the treatment or prevention of neoplasms and/or neoplasms that develop chemotherapeutic drug-resistant cancer using inhibitors of α-enolase. In addition, α-enolase therapies include nucleic acids complementary to a sequence encoding the α-enolase protein. α-enolase therapies are utilized to decrease the effective activity of α-enolase in a cancer cell, thereby increasing the sensitivity of the neoplasm to chemotherapeutic drugs. Also, the neoplastic cell's angiogenic phenotype or metastatic phenotype can be treated using nucleic acids complementary to an α-enolase coding sequence.

Examples of types of cancer and proliferative disorders to be treated with the α-enolase-targeted therapeutics of the invention include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compounds of the invention are administered to men with prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and atypical prostatic stromal lesions). The treatment and/or prevention of cancer, cancers that develop chemotherapeutic drug-resistance and/or metastatic cancer includes, but is not limited to, alleviating symptoms associated with cancer, the inhibition of the progression of cancer, the promotion of the regression of cancer, and the promotion of the immune response.

The α-enolase therapeutics can be administered in combination with other types of cancer treatments (e.g., radiation therapy, chemotherapy, hormonal therapy, immunotherapy and anti-tumor agents). Examples of anti-tumor agents include, but are not limited to, ifosfamide, paclitaxel, taxanes, topoisomerase 1 inhibitors (e.g., CPf-11, topotecan, 9-AC, and GG-211), gemcitabine, vinorelbine, oxaliplatin, 5-fluorouracil (5-FU), leucovorin, vinorelbine, Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine, and temodal. α-enolase targeting agents can be administered to a patient for the prevention or treatment of chemotherapeutic drug resistance prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of the anti-tumor agent to the subject.

α-enolase-targeted therapeutics described herein, may be administered to a subject, a mammal and a human, for the prevention or treatment of chemotherapeutic drug resistance prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of chemotherapeutic drugs described herein. Nucleic acids complementary to α-enolase messenger RNA are administered to an animal, a mammal such as a human, prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of chemotherapeutic drugs. The nucleic acids can be incorporated into a liposome for transport into a cell.

α-enolase-targeted therapeutics can be administered by a variety of mechanisms that are known in the art. Therapies can be administered during an open surgical procedure in which the physician places the therapy into direct contact with the tumor. Therapies can be administered in the form of an aerosol or vapor through an inhaler. Alternatively, a patient can be intubated, and the α-enolase-targeted therapeutics can be placed into the patient through the tube. The above-described means are not meant to be limiting. Any means can be utilized to treat a patient so long as the α-enolase-targeted therapeutics are introduced to the patient such that the therapies can contact the cancerous growth or tumor.

1.9 Pharmaceutical Formulations and Methods of Treatment

The present invention provides for both prophylactic and therapeutic methods of treating a subject having cancer and/or cancers that have developed chemotherapeutic drug-resistance. Furthermore, the present invention provides both prophylactic and therapeutic methods directed to treating a subject that has developed metastatic neoplastic disease. Administration of a prophylactic agent can occur prior to the manifestation of symptoms characteristic of the neoplasm, such that development of the neoplasm is prevented or, alternatively, delayed in its progression. In general, the prophylactic or therapeutic methods comprise administering to the subject an effective amount of a compound which comprises a α-enolase binding component that is capable of binding to α-enolase present in neoplastic, and particularly chemotherapeutic drug-resistant neoplastic, cells and which compound is linked to a therapeutic component. The α-enolase binding component or agent binds to the α-enolase expressed in the neoplastic cells and prevents α-enolase activity in the cells, thereby rendering the cells more susceptible to a chemotherapeutic treatment.

α-enolase can be targeted to neoplastic cells using a variety of targeting means. In some instances, the targeting component can be an antibody that binds to a neoplastic cell marker. The α-enolase binding component can be targeted to the neoplastic cells by vimentin, nucleophosmin or HSC70 antibodies, for example. Examples of α-enolase targeting components include monoclonal anti-vimentin antibodies and fragments thereof. Subsequent to α-enolase internalization into a neoplastic cell, therapeutic components can be administered to a patient to kill the neoplastic cell. Examples of suitable therapeutic components include traditional chemotherapeutic agents such as Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, and Vinorelbine.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is used, including intramuscular, intravenous, intraperitoneal, and subcutaneous. For injection, the compounds of the invention can be formulated in liquid solutions, in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as targeting agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in a conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology utilizes microspheres of precapillary size which can be injected via a coronary catheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a therapeutic and gene delivery system for the α-enolase-targeted therapeutic can be introduced into a patient by any of a number of methods, each of which is familiar in the art. For instance, a pharmaceutical preparation of the α-enolase-targeted therapeutic can be introduced systemically, e.g., by intravenous injection.

The pharmaceutical preparation of the α-enolase-targeted therapeutic compound of the invention can consist essentially of the compound in an acceptable diluent, or can comprise a slow release matrix in which the gene delivery vehicle or compound is imbedded.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

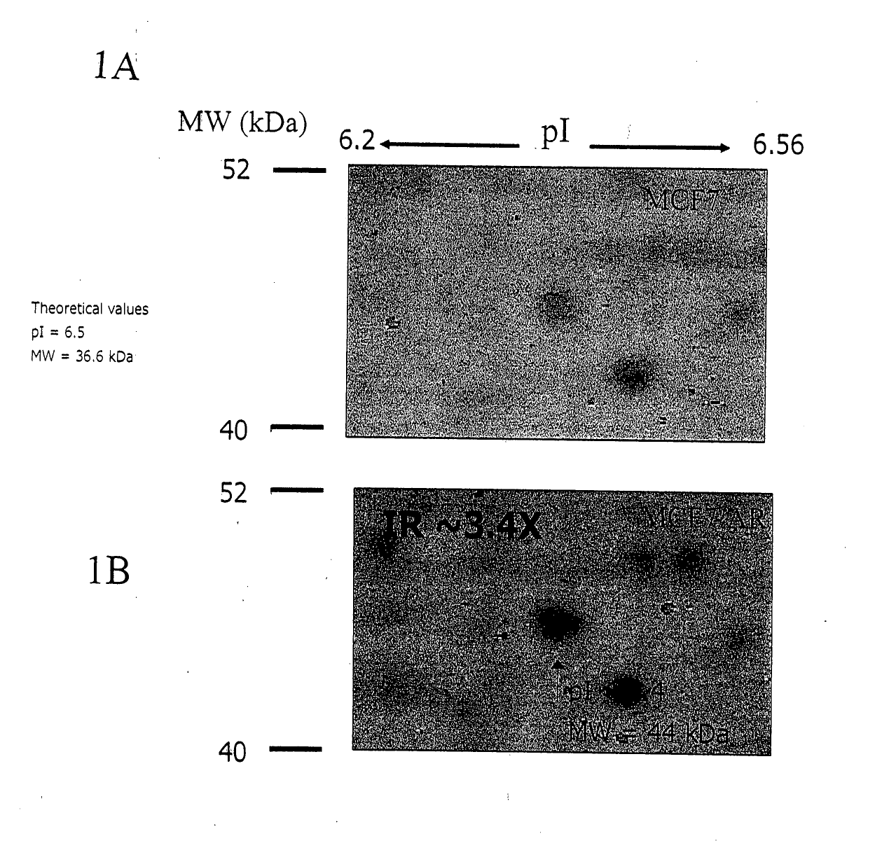

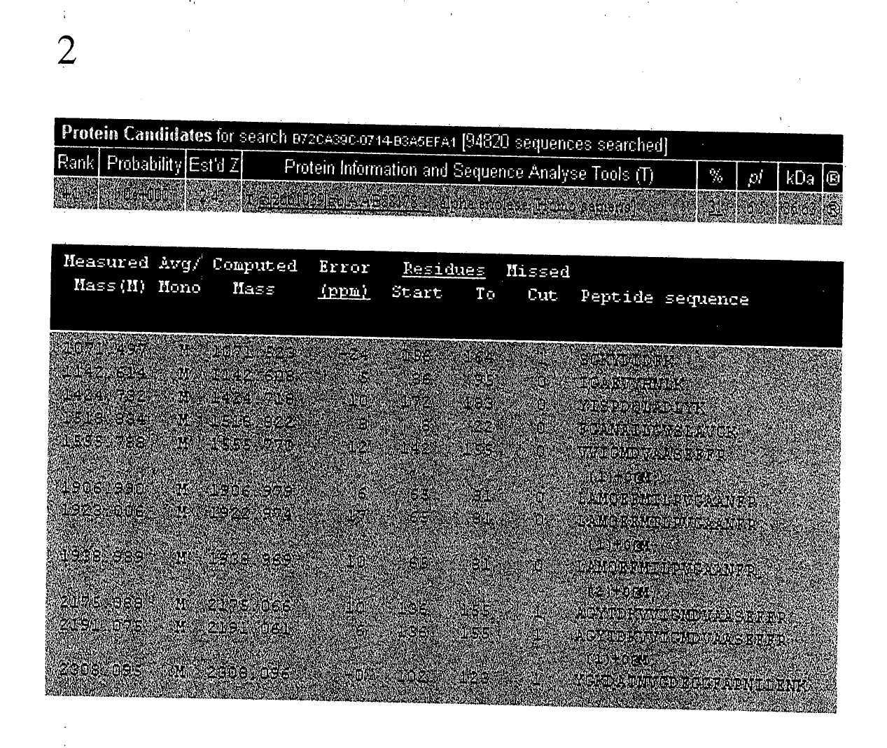

To demonstrate the methods according to the invention, an α-enolase targeting agent was prepared and tested for its ability to increase the sensitivity of various cancer cell samples to chemotherapeutic drugs. For example, the well-characterized MCF-7 breast cancer cell line and its doxorubicin resistant counterpart MCF-7/AR were used to determine the potential overexpression of MDR proteins in drug-resistant cancer cells. Two-dimensional gel electrophoresis was performed on cell samples. The results were shown in FIGS. 1A and 1B. Mass spectrometry of the resulting peptide fragments showed extensive homology to α-enolase (FIG.

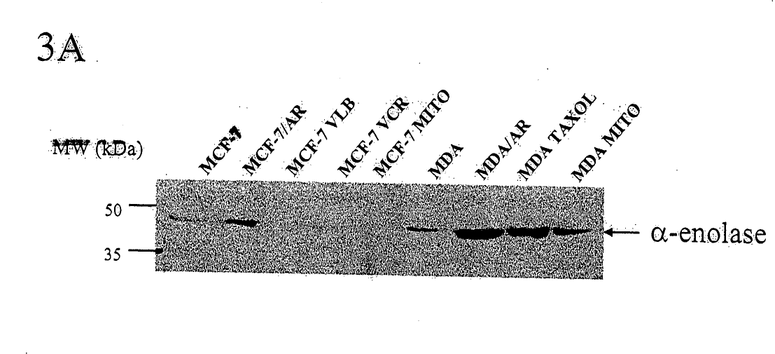

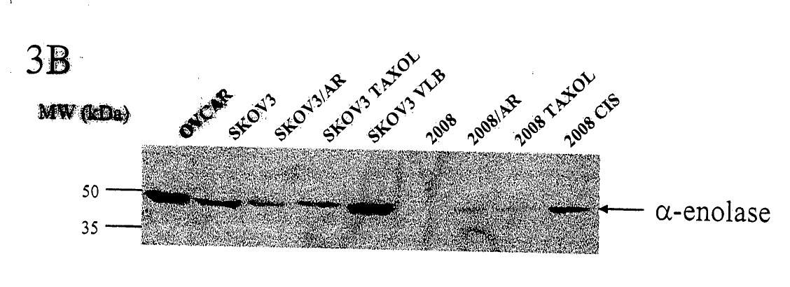

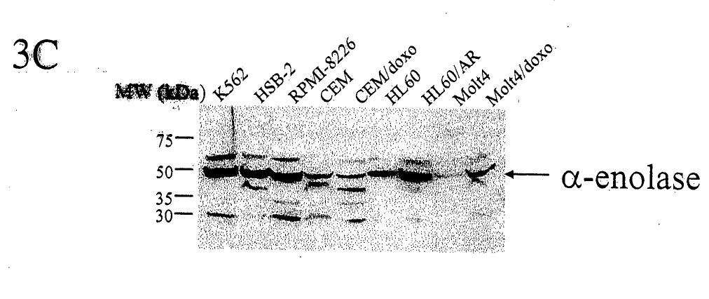

In addition to determining the expression of α-enolase protein in MCF-7 cell lines, α-enolase protein expression was measured in a panel of different drug-resistant breast cancer cell lines using Western blot analysis. Alpha-enolase was strongly induced in adriamycin-resistant breast cancer cell lines MCF-7 and MDA-MB231. Elevated levels of this protein were also found in the taxol-resistant MDA cells (FIG. 3A). Elevated levels of α-enolase were also found in a vinblastin-resistant SKOV3 cell line and cisplatinum-resistant cell lines (FIG. 3B). In addition, enolase expression was elevated in adriamycin-resistant lung cancer cell lines H69 and H460 (FIG. 3C) and in certain leukemic cell lines (K562, HSB-2 and RPMI-8226; FIG. 3D).

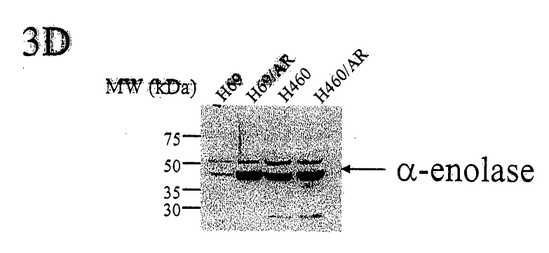

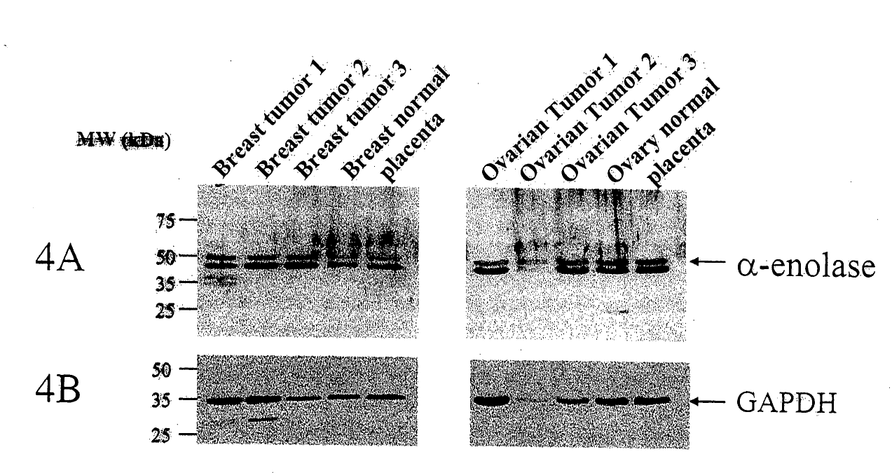

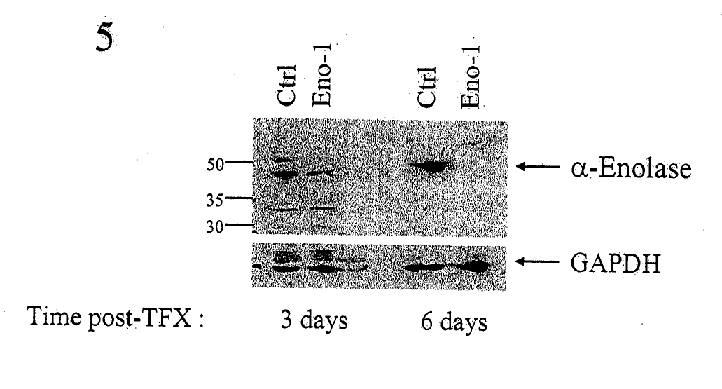

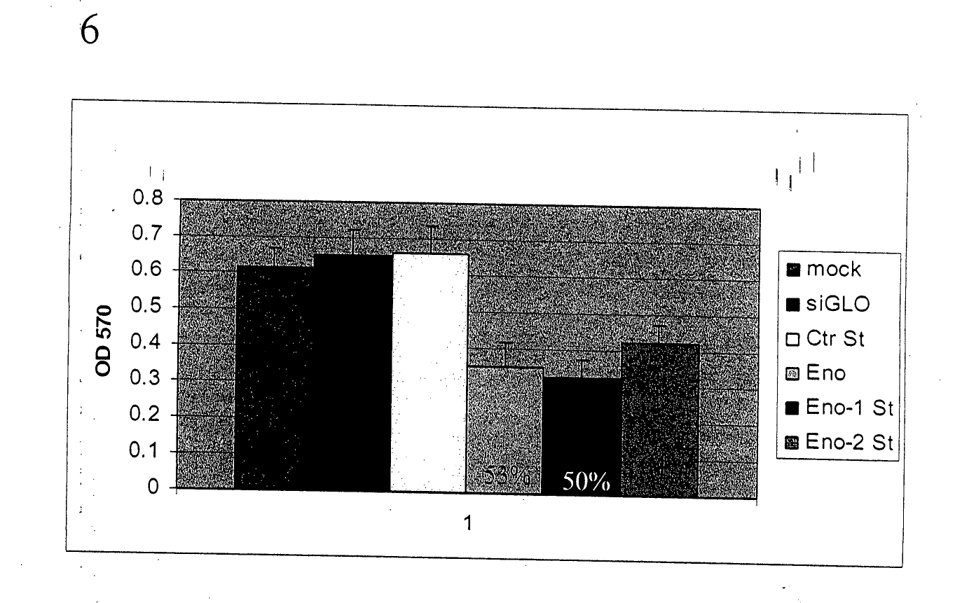

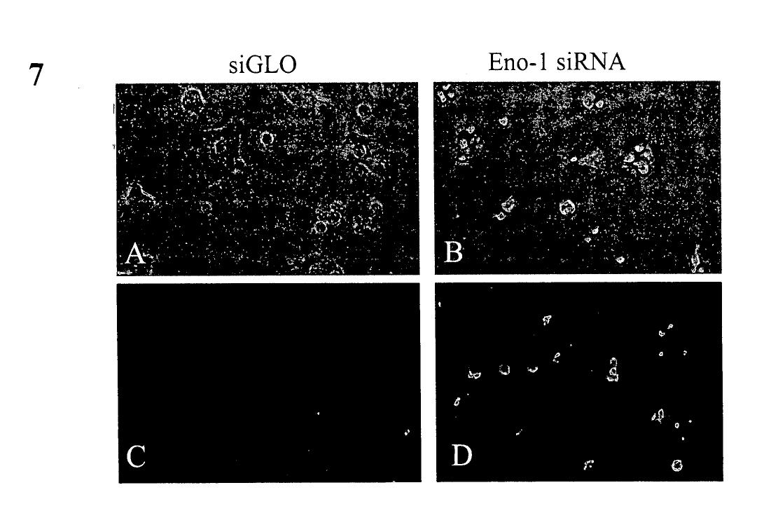

As shown in FIG. 4, elevated levels of α-enolase were observed in three different breast tumors as compared to normal breast tissue. In order to further investigate the potential involvement of an α-enolase in drug resistance, RNAi technology was employed to silence its expression in breast and ovarian cancer cells. The siRNA duplex was effective in decreasing α-enolase protein expression by 50-80% in a variety of cell lines (FIG. 5). Cell viability was also determined for breast tumor cells, MCF-7, by MTT assay, and a significant impact was detected in the α-enolase silenced cells. Similar effects on cell morphology and viability were observed with all three enolase-specific siRNA duplexes but not with control siRNAs (FIG. 6). Annexin-V staining was used to determine the effects of siRNA α-enolase on MCF-7 cells compared to cells treated with a control siRNA (FIGS. 7A and 7B). Annexin-V positive cells were detected in the α-enolase siRNA transfected MCF-7 cells compared to baseline levels detected in cells treated with a control siRNA alone (FIGS. 7C and 7D).

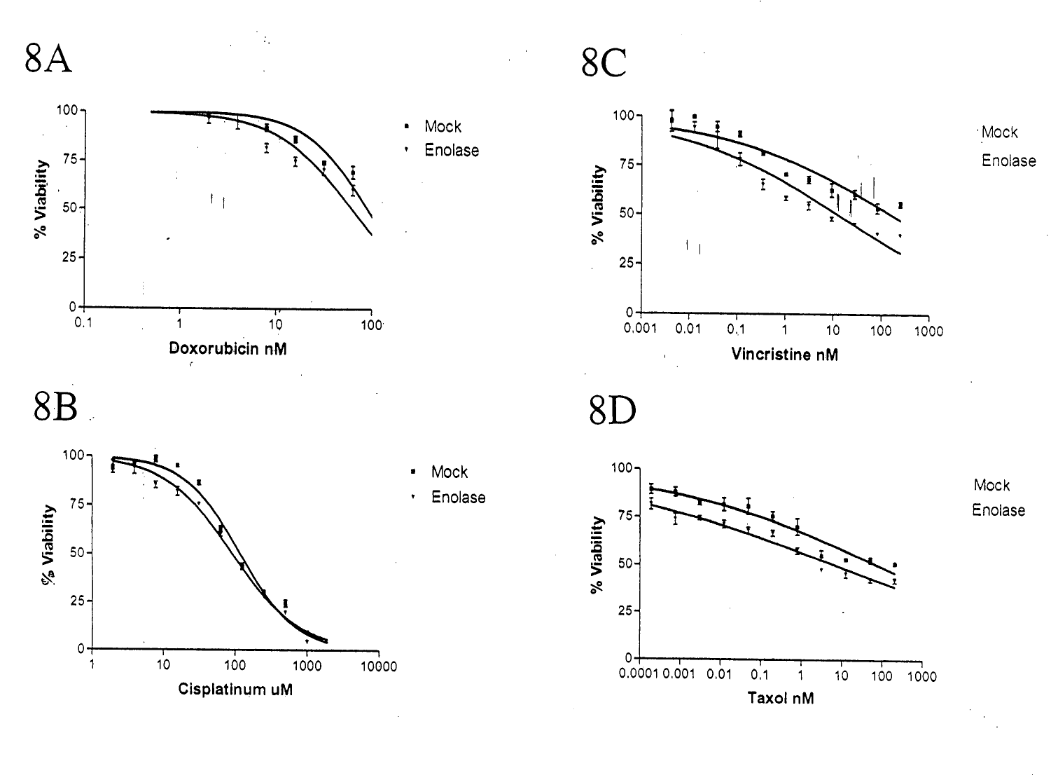

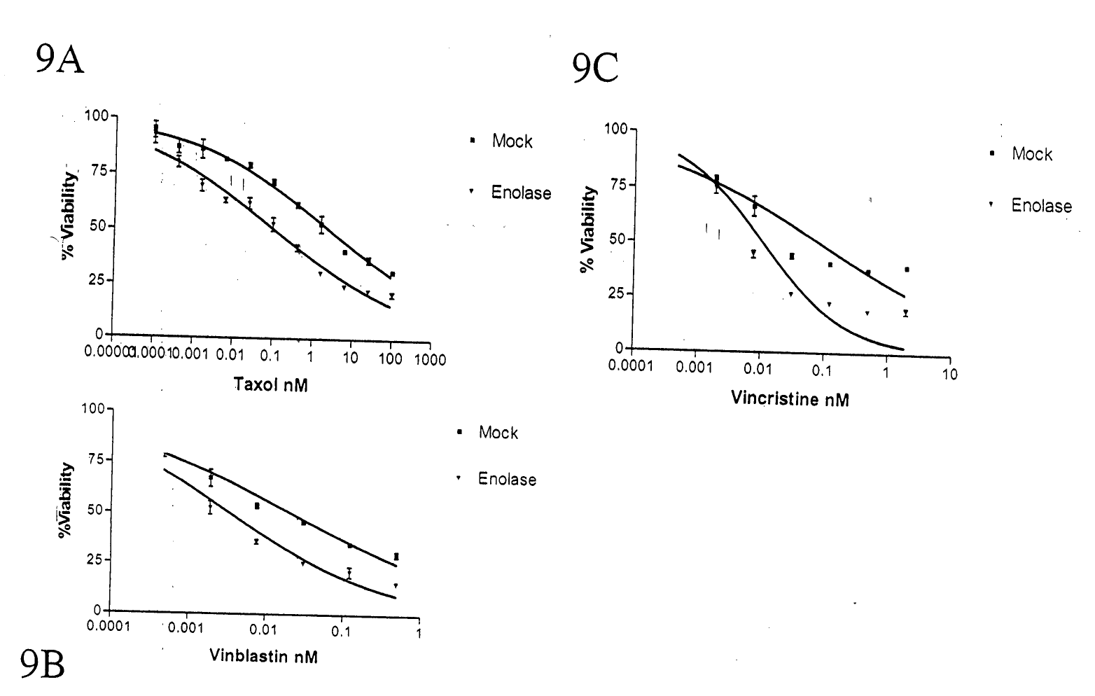

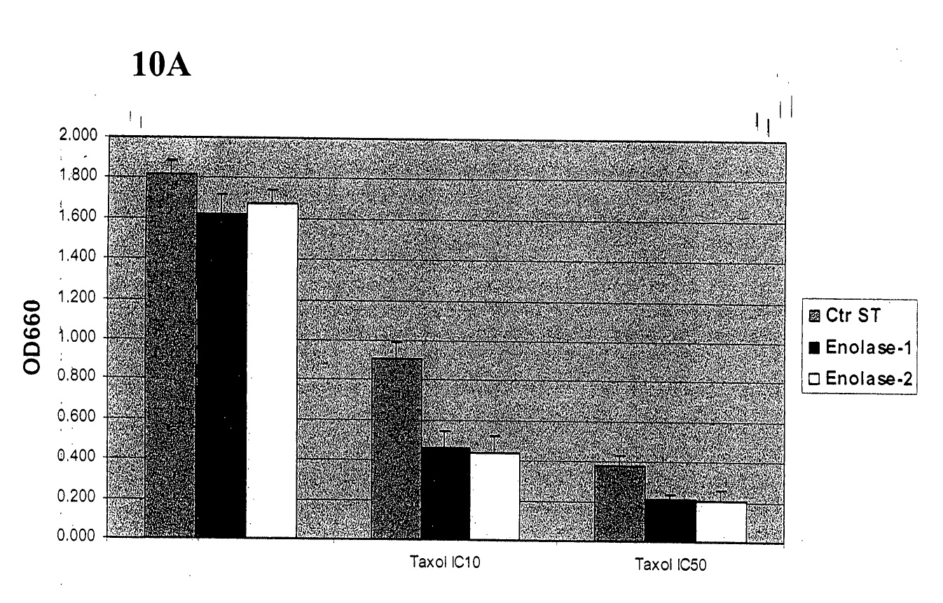

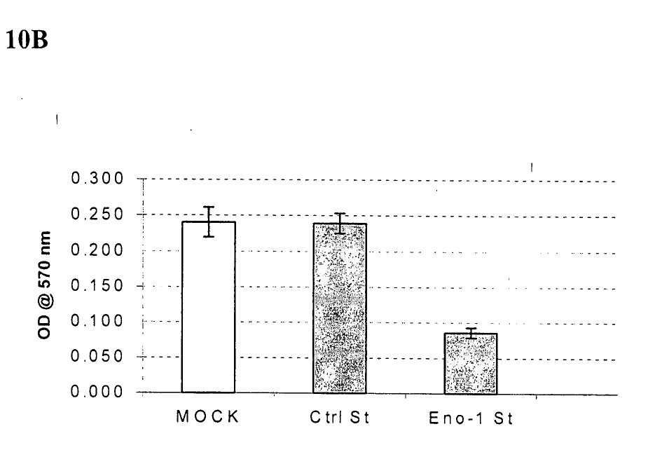

To evaluate the effect of α-enolase depletion on the chemosensitivity of cells to cytotoxic agents, MTT assays were performed on MCF-7 cells transfected with α-enolase-specific siRNAs. Furthermore, the α-enolase silenced MCF-7 cells displayed an increase in their sensitivity to taxol and vincristin as evidenced by the cytotoxicity graphs (FIGS. 8C and 8D). The predicted EC₅₀ values for the α-enolase-depleted cells were on average 10-fold lower for taxol and 12-fold lower for vincristin as compared to control siRNA treated MCF-7 cells. The same procedure was used for these drugs as detailed above. Similar results to those observed in MCF-7 were obtained in α-enolase-depleted CaOV3 ovarian carcinoma cell line, i.e., drug sensitivity to taxol and Vincristin (FIGS. 9A-9C). The enhanced cytotoxic effect with taxol and vincristin was also seen in SKOV3 cells (data not shown).

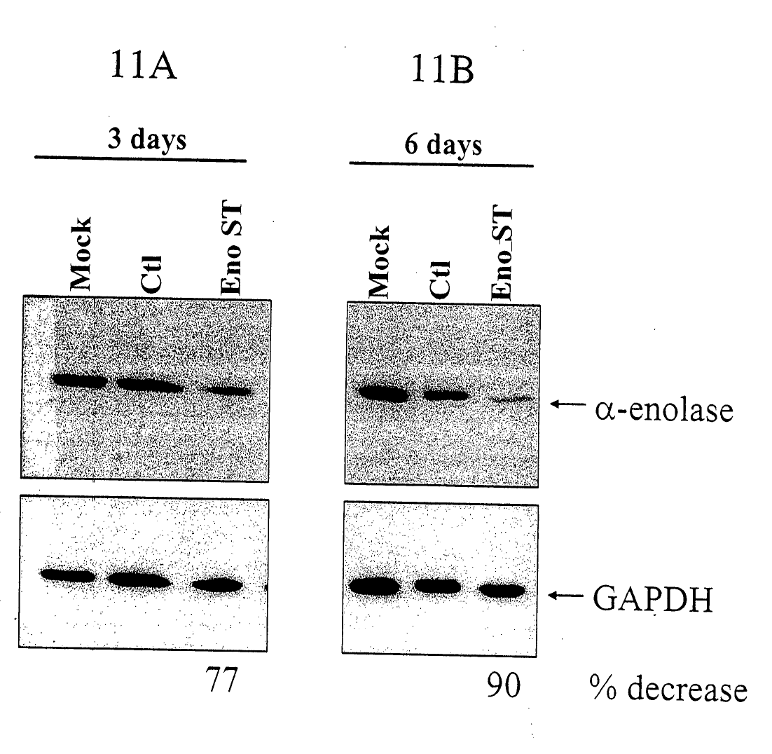

To determine the impact α-enolase siRNA silencing has on growth and differentiation of tumor cells, an assay monitoring clonogenic cell growth was employed. Alpha-enolase siRNA transfected cells were allowed to grow and form colonies for 7 days in the presence or absence of taxol. The results are shown in FIG. 10A. Furthermore, viability of adriamycin-resistant MCF-7 cells was determined to be 65% less than that of siRNA untreated cells (FIG. 10B).

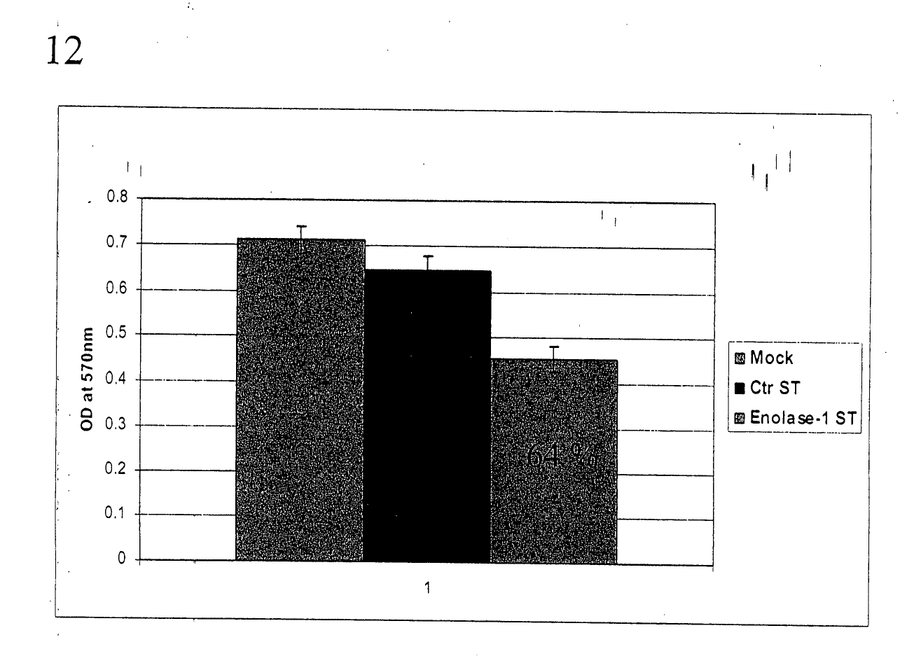

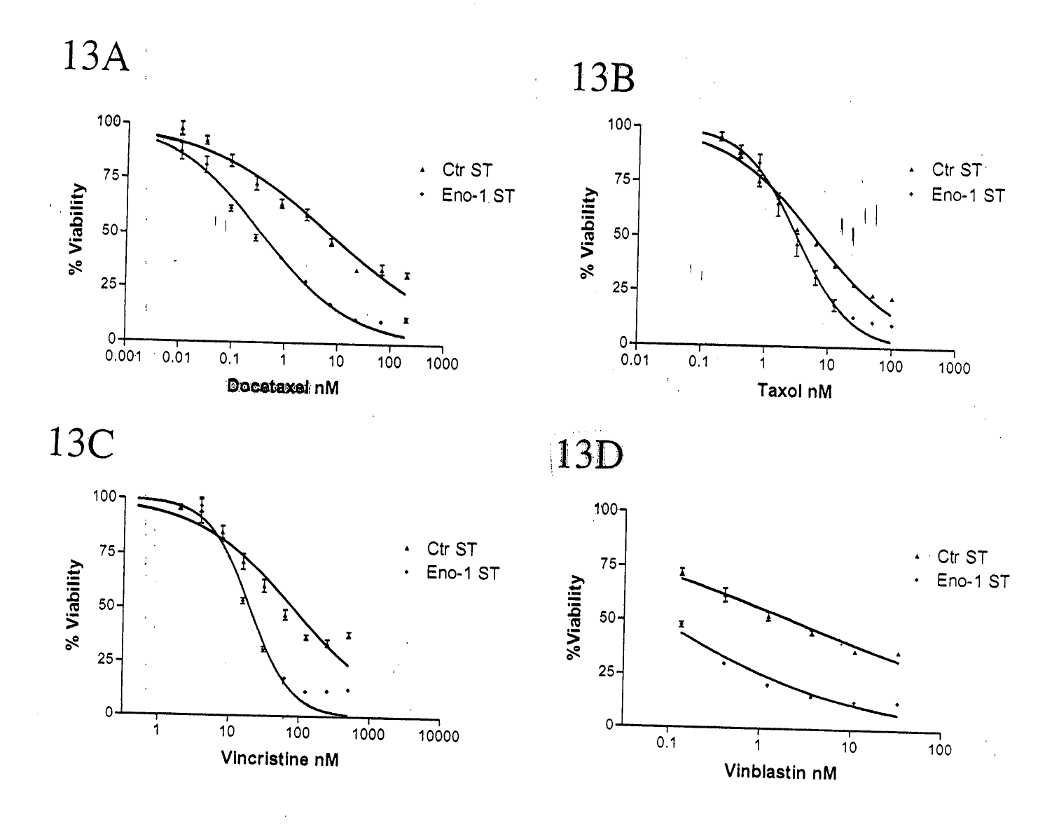

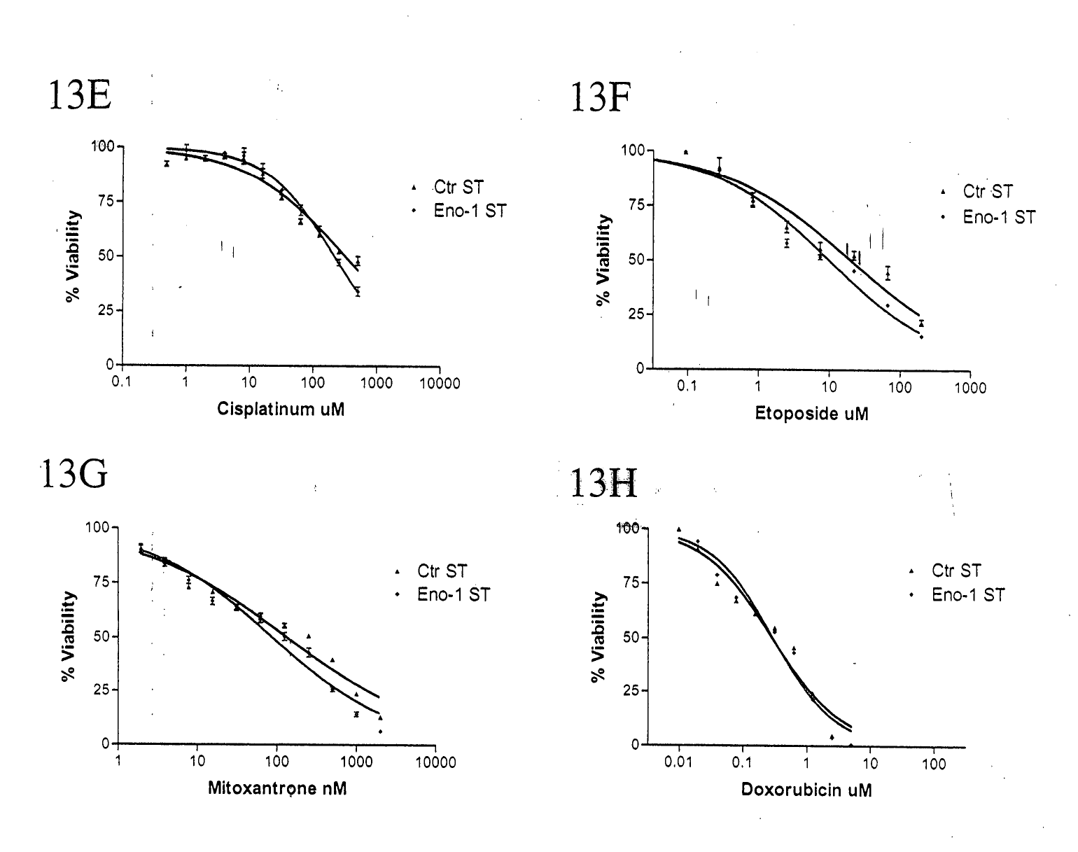

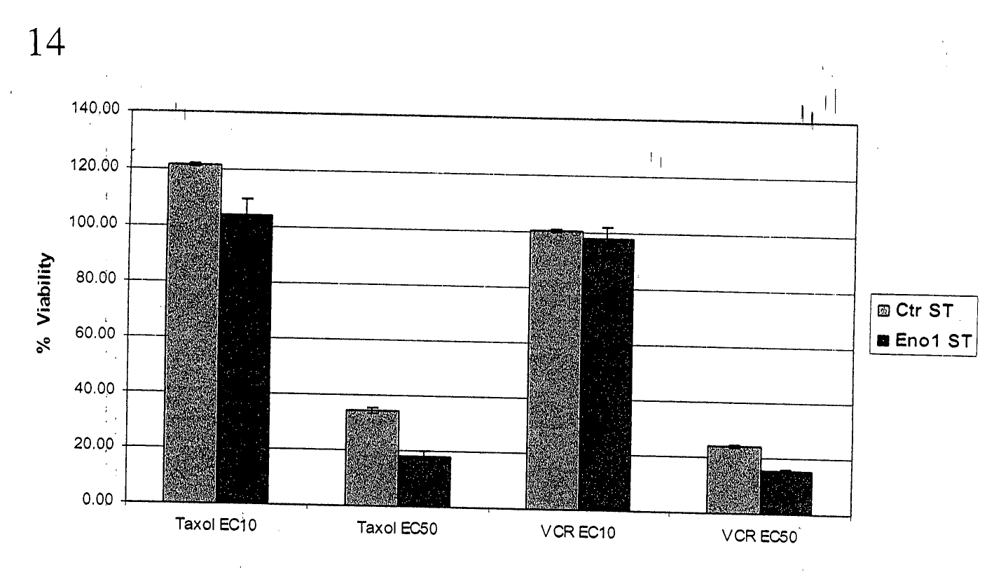

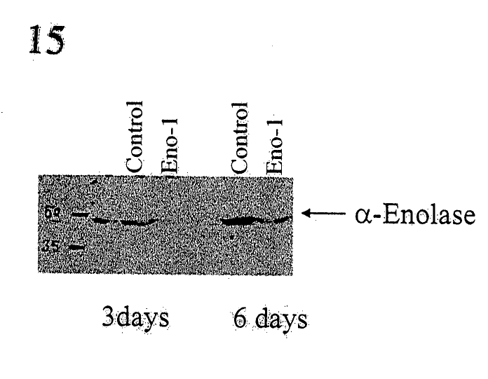

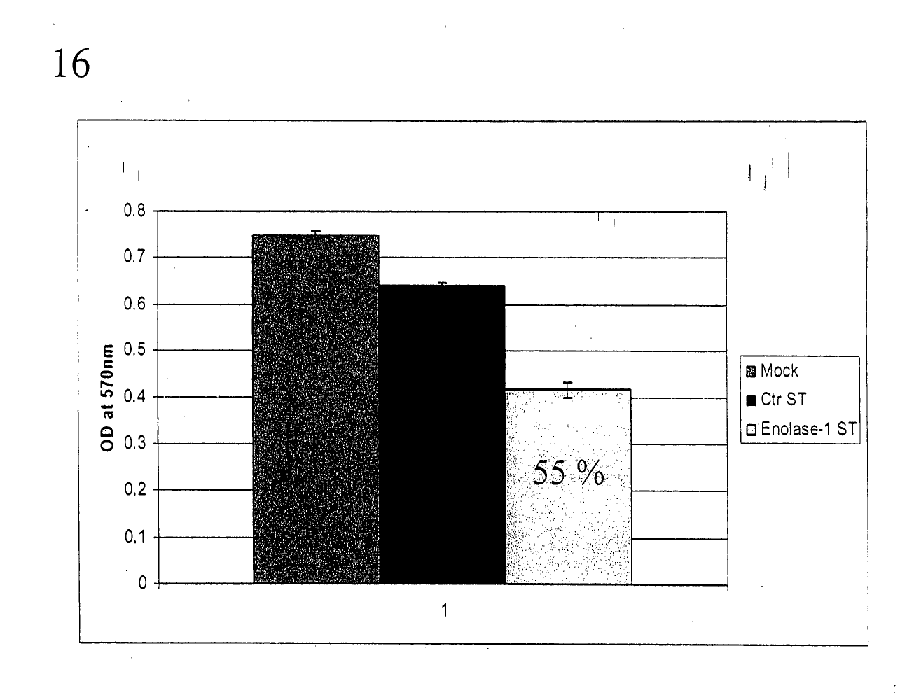

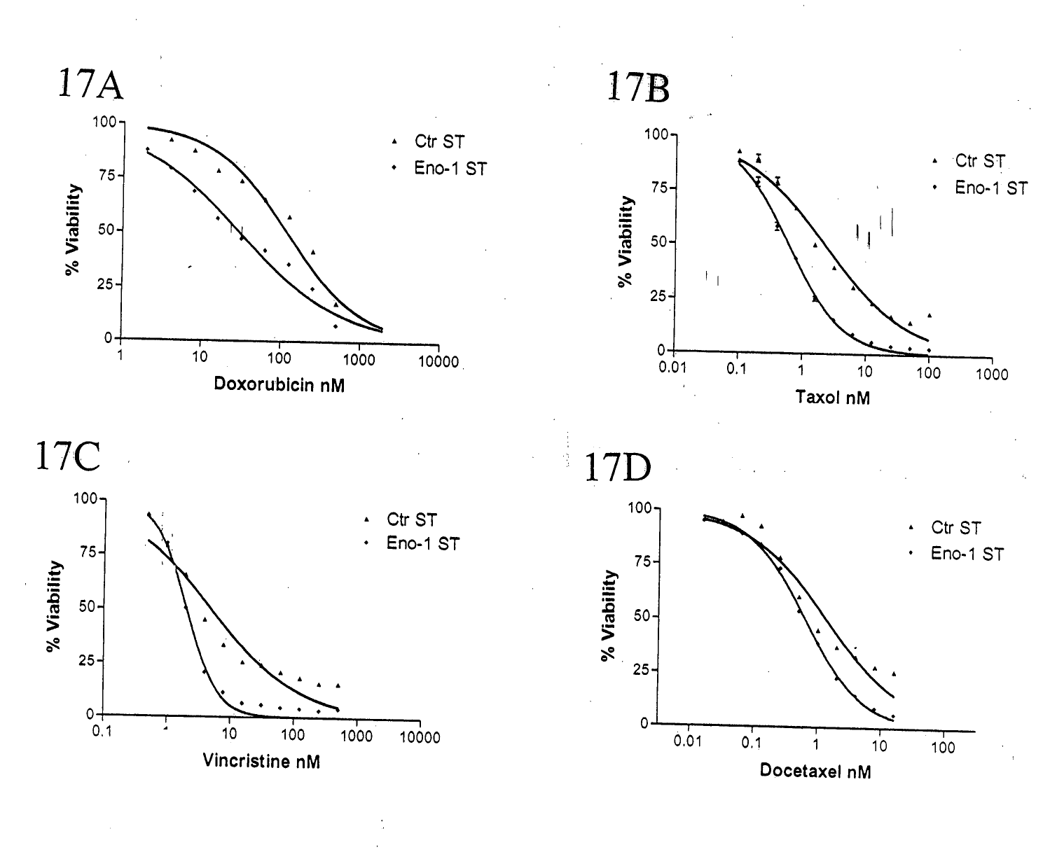

The A549 cell line, which was derived from a non-small cell carcinoma of the lung, was used for α-enolase silencing experiments. Cell transfectants were produced as described below and α-enolase protein expression was determined by Western blot analysis (FIGS. 11A and 11B). The effect of mock siRNA expression versus α-enolase siRNA expression on cell viability is demonstrated in FIG. 12. Alpha-enolase directed siRNA reduced α-enolase expression by 77% three days post-transfection, and by 90% six days post-transfection. Only α-enolase silencing decreased the viability of A549 cells, while mock transfections and expression of control siRNA showed little or no effects on viability (FIG. 12). Moreover, cell survival was reduced in α-enolase-silenced A549 cells treated with different chemotherapeutic drugs (FIGS. 13A-13H). In particular, enhanced chemosensitivity was demonstrated in A549 cells treated with α-enolase siRNA to drugs such as docetaxel, taxol, vincristine, and vinblastin (FIGS. 13A-13D). Cells treated with etoposide and mitoxantrone also showed increased sensitivity to these treatments, albeit more subtly than in A549 cells treated with docetaxel, taxol, vincristine, or vinblastin (FIGS. 13F and 13G).

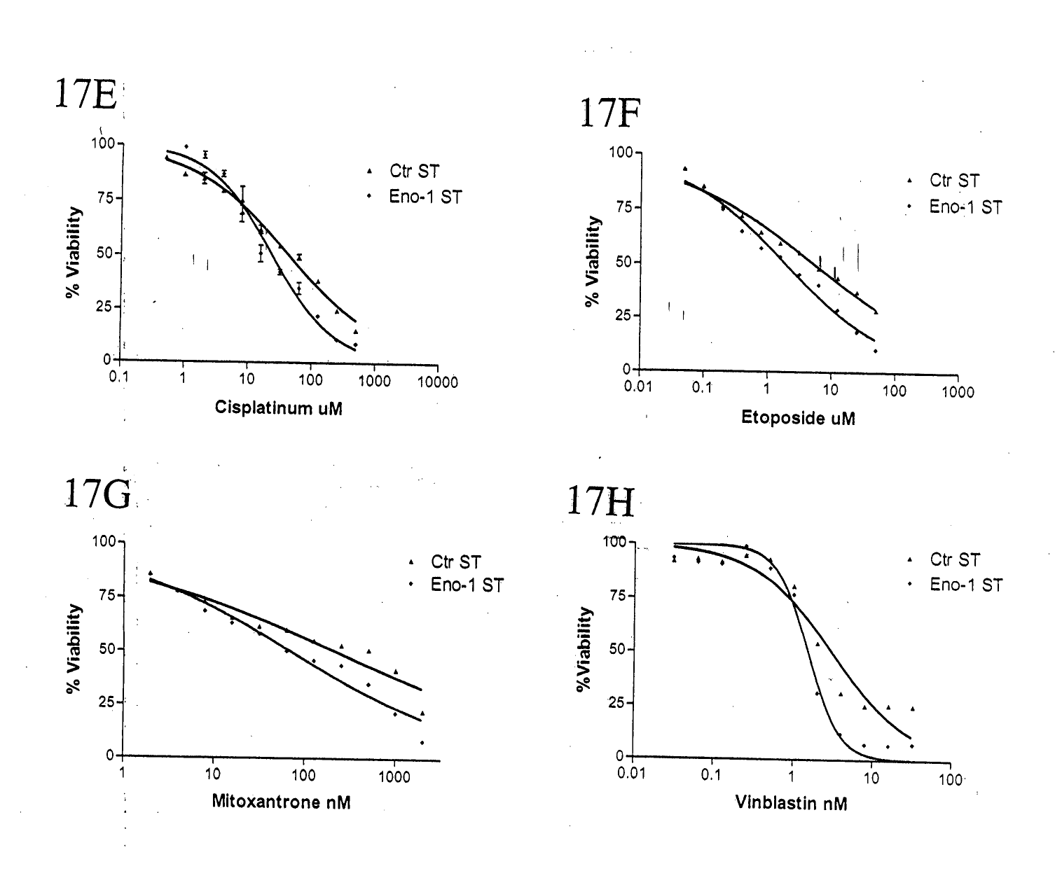

Further confirming the effects of α-enolase silencing on chemotherapeutic drug resistance, clonogenic assays established that cells transfected with vectors expressing α-enolase siRNA were more sensitive to taxol or vincristin treatment than cells transfected with control vectors (FIG. 14). In particular, cells expressing α-enolase siRNA were less viable than control cells when challenged with taxol in their EC₁₀ and EC₅₀ values. Furthermore, cells expressing α-enolase siRNA were less viable than controls in their EC₅₀ values when challenged with vincristin.

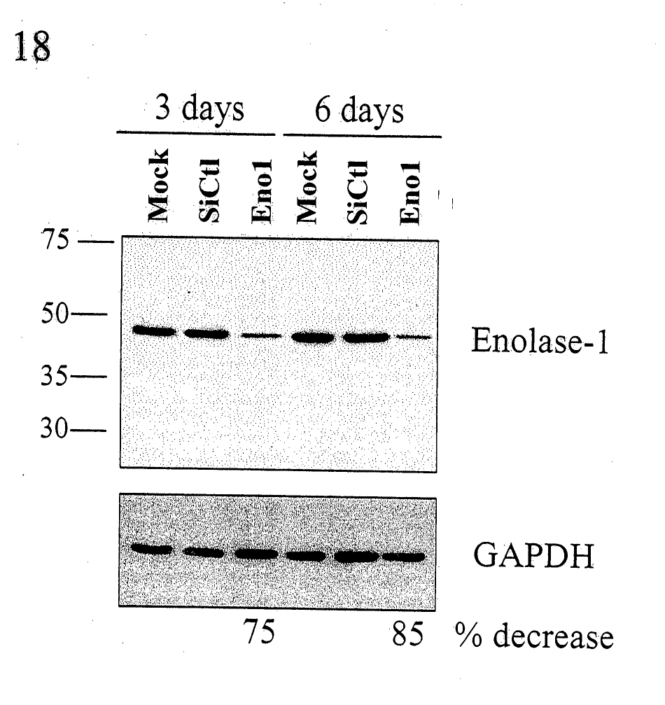

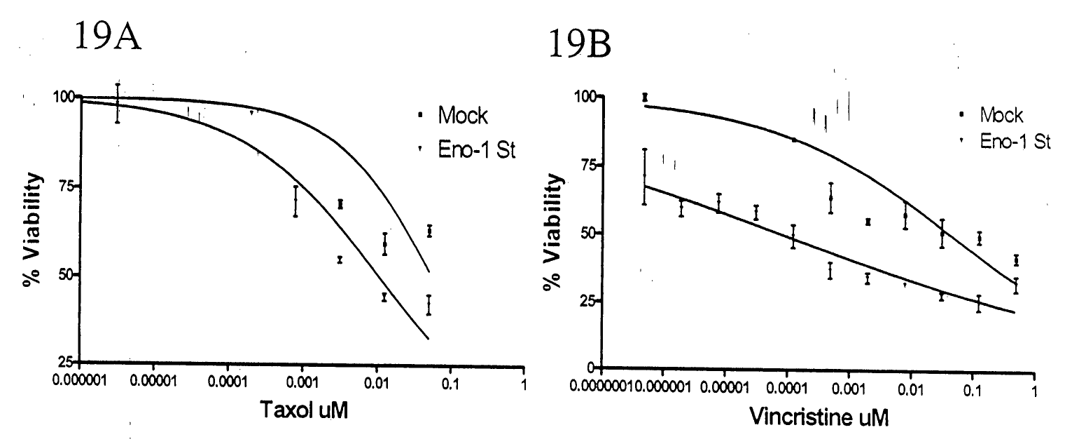

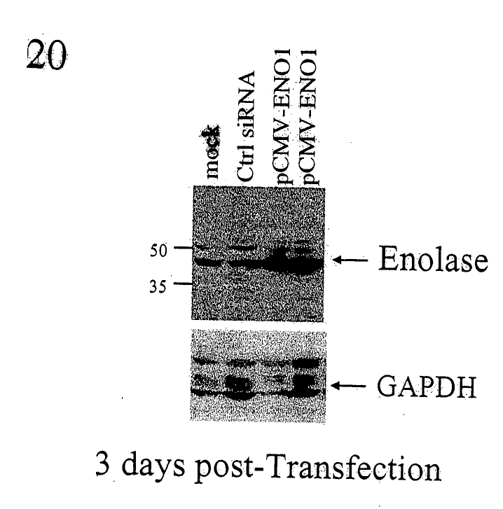

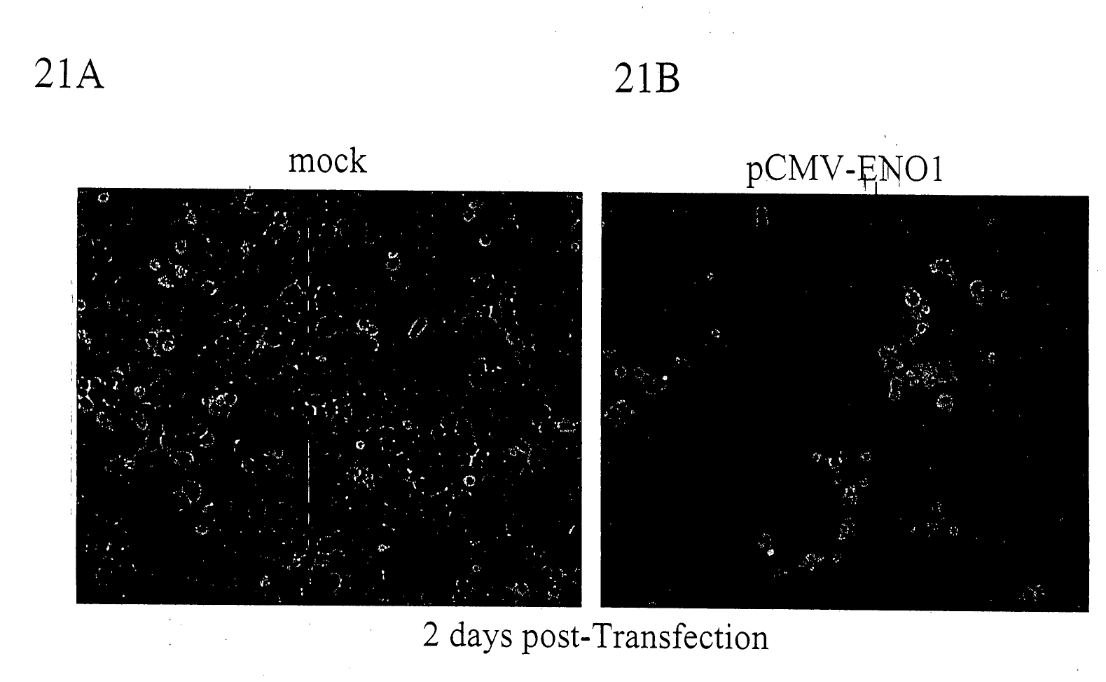

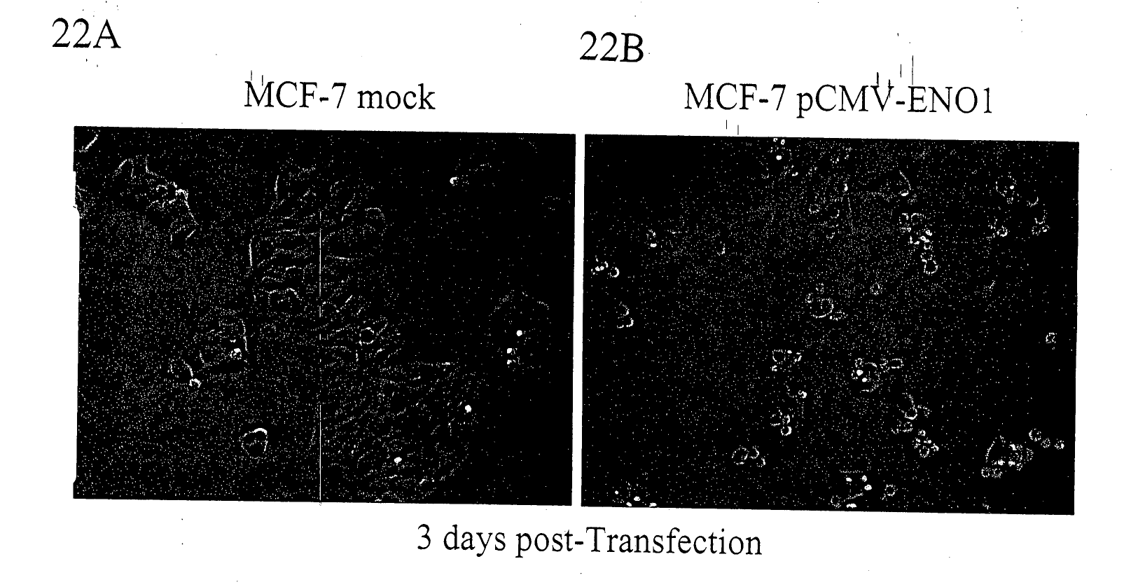

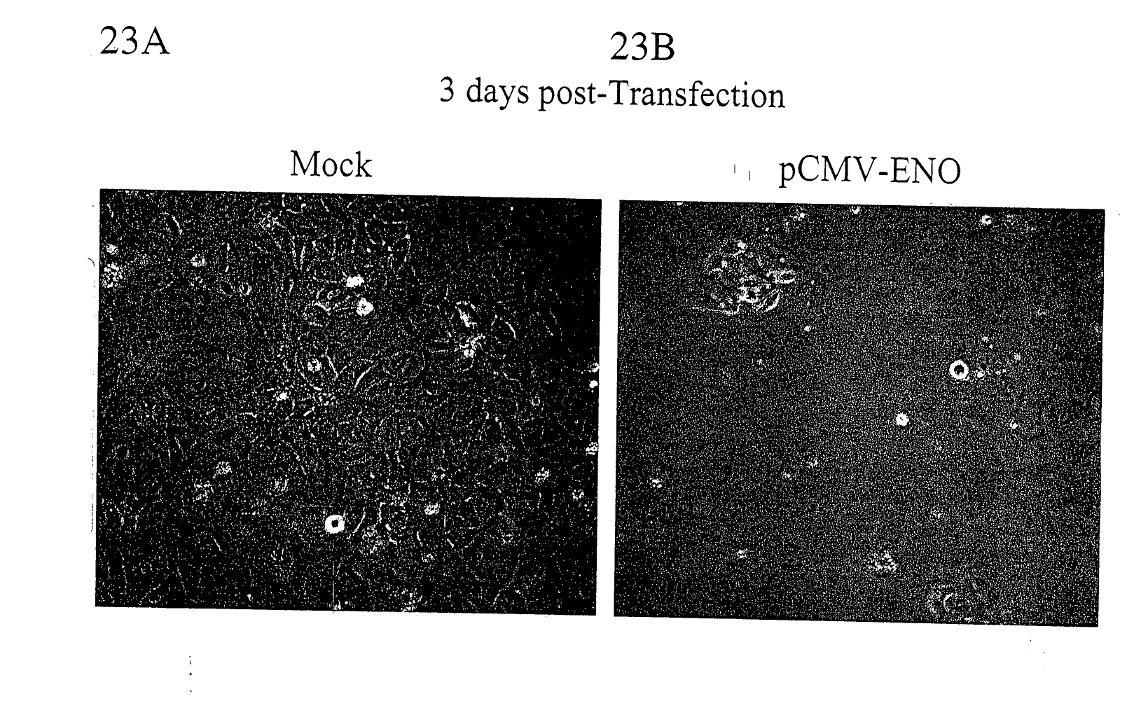

Alpha-enolase siRNA silencing studies were also performed on the non-small cell lung carcinoma cell line H460. Western blot experiments confirmed that α-enolase expression was decreased by α-enolase-targeted siRNA, while control siRNA did not affect the expression of α-enolase protein (FIG. 15). MTT assays established that the H460 α-enolase siRNA transfectants were less viable than mock-transfected cells or cells transfected with control siRNA (FIG. 16). In addition, α-enolase silencing decreased the viability of cells undergoing chemotherapeutic drug treatment with several different compounds including doxorubicin, taxol, vincristin, docetaxel, cisplatinum, etoposide, mitoxantrone and vinblasin, as compared to control cells undergoing the same treatment regimen (FIG. 17A-H, respectively). In particular, α-enolase-silenced cells treated with doxorubicin and taxol showed the greatest decreases in viability when compared to controls (FIGS. 17A and B). These results indicate that α-enolase silencing adversely affects the viability of H460 cells.

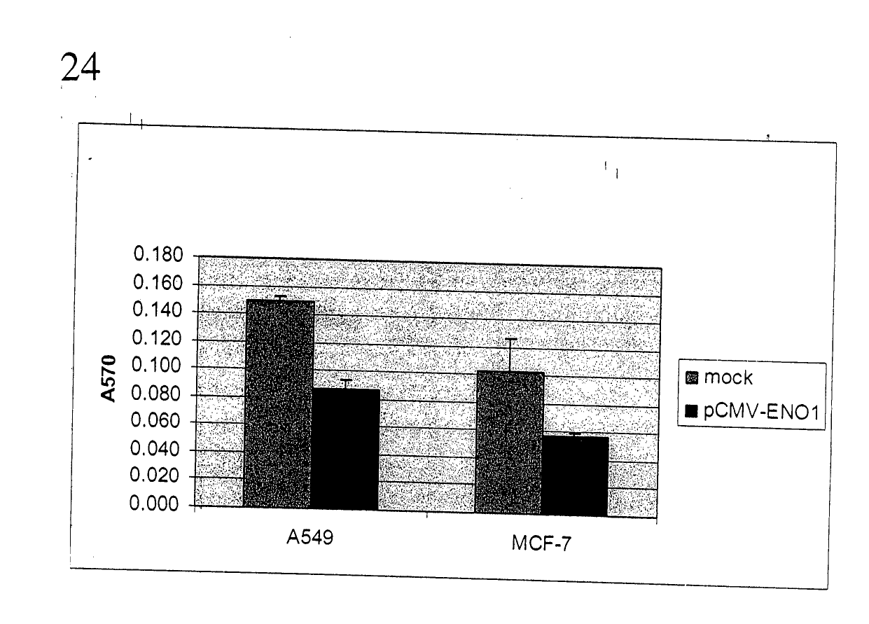

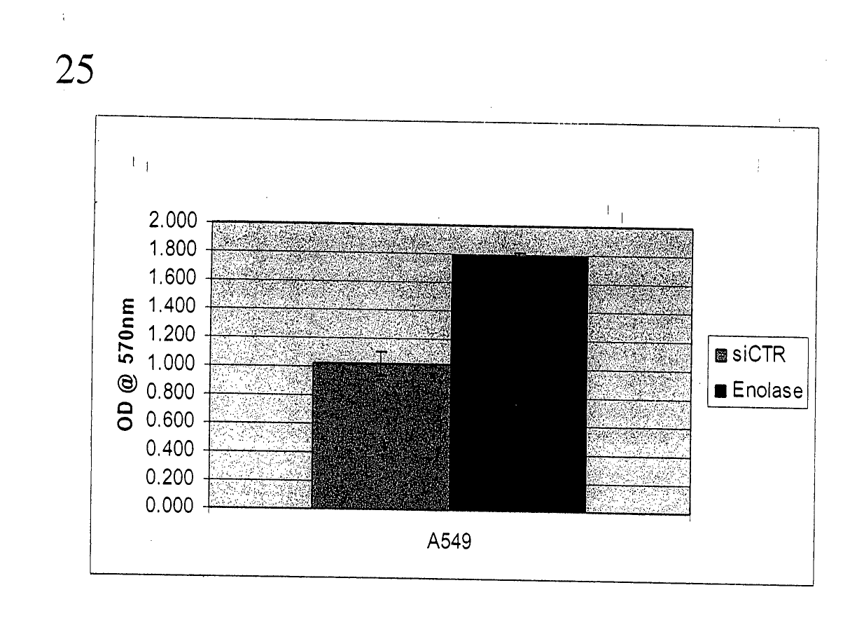

Moreover, α-enolase expression was successfully reduced in SW-480 cells treated with α-enolase siRNA (FIG. 18). When assayed by Western blot, protein levels of enolase remained decreased for at least 6 days post-transfection (FIG. 18). Alpha-enolase silencing resulted in decreased viability of SW-480 cells, while SW-480 cells expressing α-enolase showed no reduction in viability (FIGS. 19A and 19B). In addition, α-enolase-depleted cells showed greater chemosensitivity to taxol and vincristin than control cells. For example, enolase-depleted cells were approximately 210 times more sensitive to vincristin than mock transfected cells (FIG. 19B).

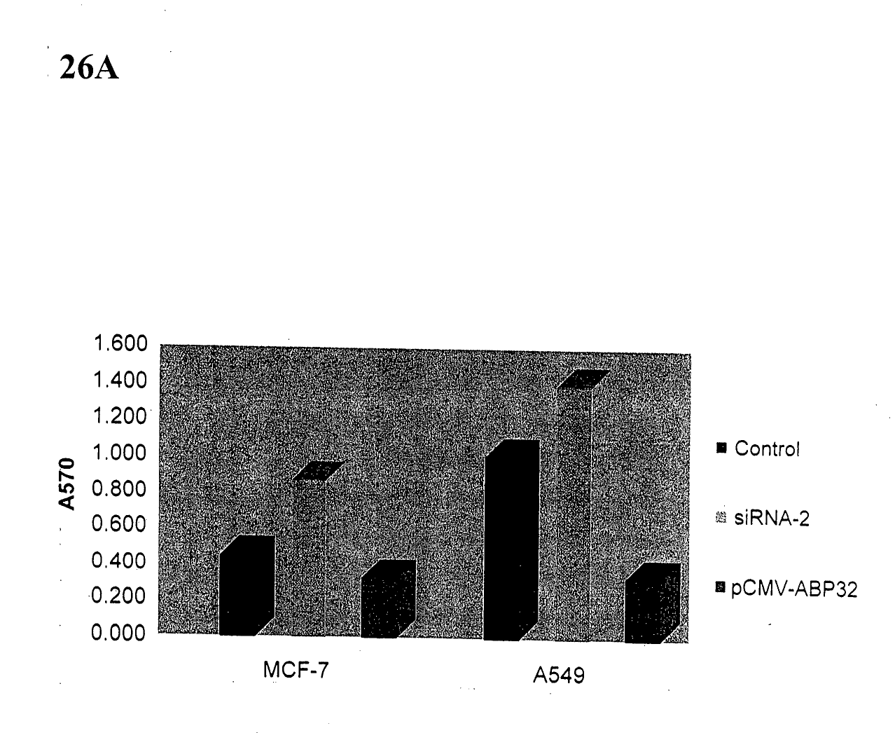

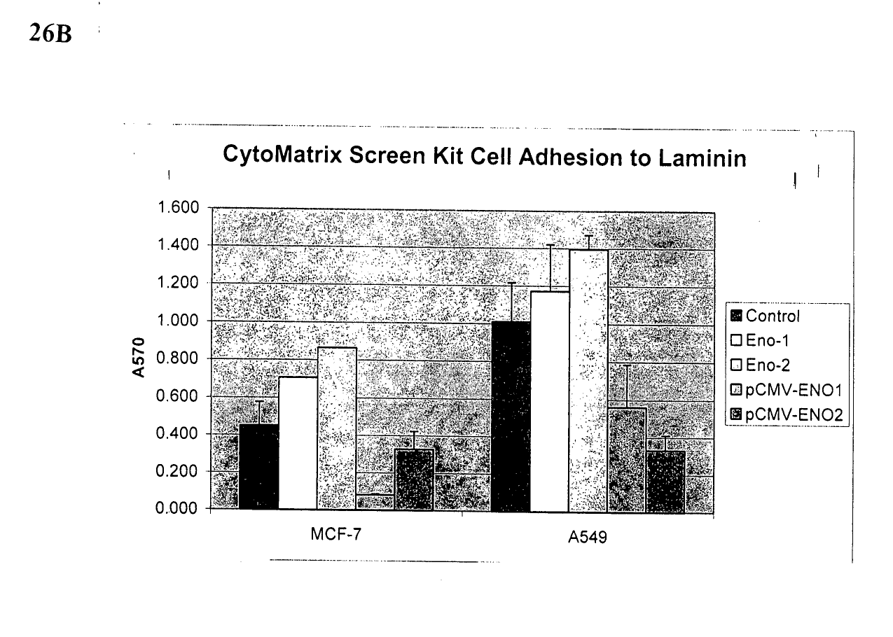

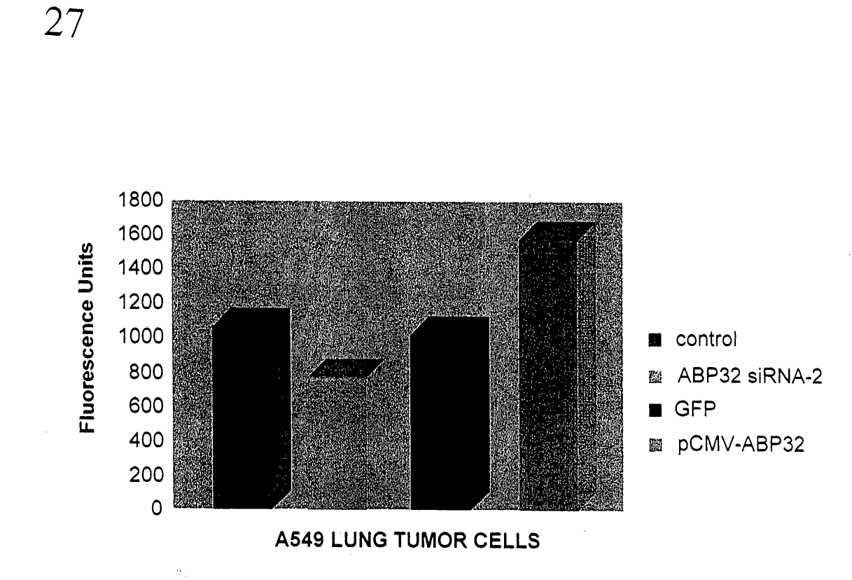

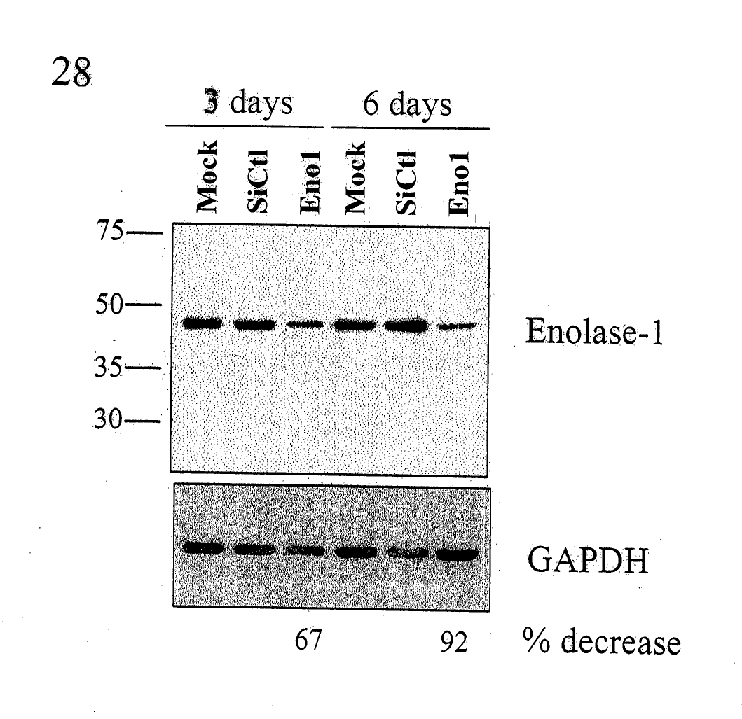

Cell adhesion studies were also performed on cells overexpressing α-enolase to determine the importance of α-enolase to cell adhesion. Cell adhesion is a normal characteristic of cells that is lost when cells become metastatic, thereby allowing cancer cells to loosen from a tumor and migrate to other sites in the organism (see, e.g., Furuta et al. (2005) Melanoma Res. 15(1):15-20). Overexpression of α-enolase in the lung cancer cell line H460 was accomplished by transfecting the pCMV vector containing the human α-enolase cDNA into the line. The resulting overexpression of α-enolase was confirmed by Western blot analysis in MCF-7 cells transfected with the pCMV vector containing the full-length human α-enolase coding sequence (FIG. 20). The results shown in FIG. 21B demonstrate that increased α-enolase expression allowed the H460 cells to become unattached to other cells within the medium. The control cells transfected with a mock vector showed unaltered cell-cell adhesion, forming a confluent monolayer on the surface of the dish (FIG. 21A). Similar results were also found for MCF-7 cells and A549 cells overexpressing α-enolase (FIGS. 22A-22B and FIGS. 23A-23B, respectively).

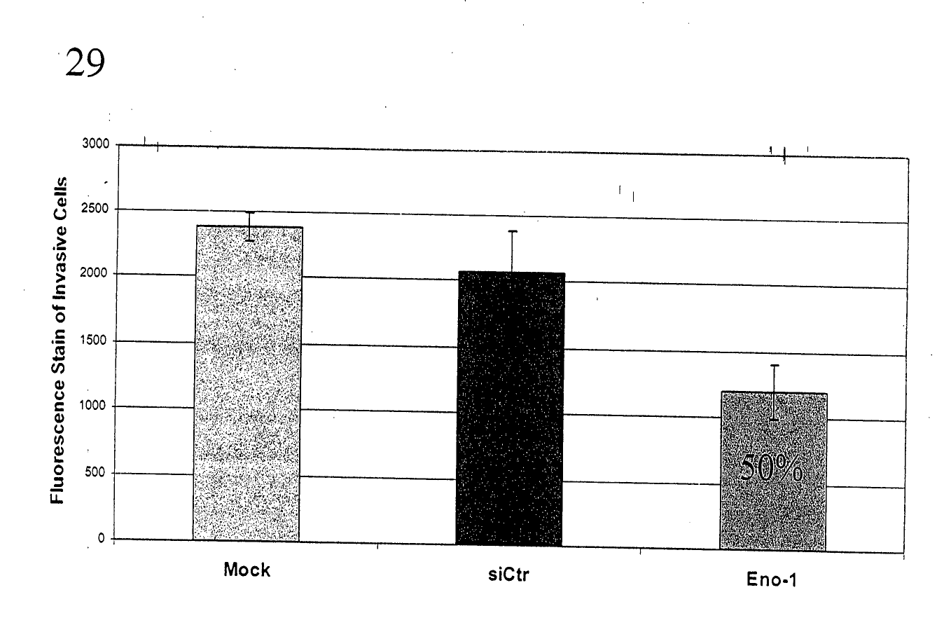

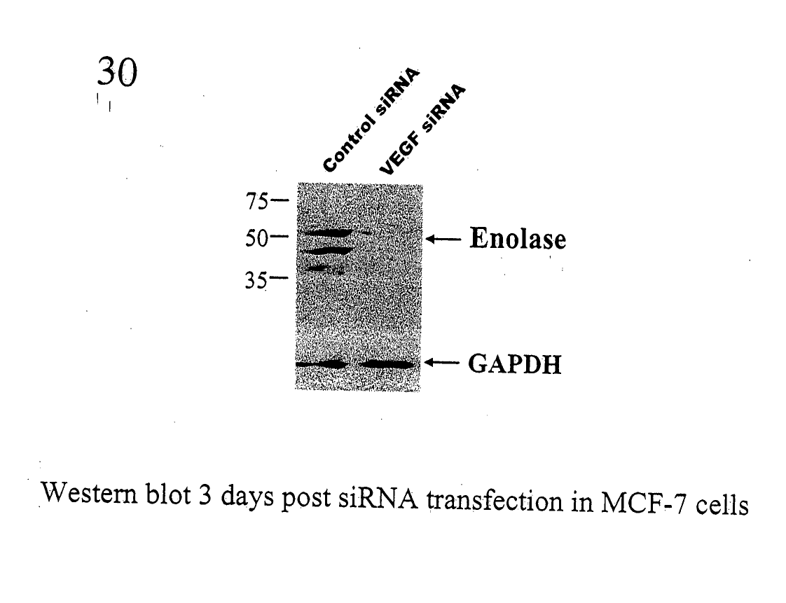

As shown in FIG. 24, non-small cell lung carcinoma cells A549 adhesion to laminin-coated multi-well plates was impaired by the overexpression of α-enolase. Cells overexpressing α-enolase adhered to the laminin-coated surface at half the rate of mock-transfected cells. These results were further confirmed by experiments showing that α-enolase silencing enhanced cell adhesion of A549 cells to collagen-coated plates (FIG. 25). A549 cell adhesion was increased by 40% compared to cells expressing normal levels of α-enolase (FIG. 25). FIGS. 26A and 26B is a summary of the effects of α-enolase overexpression and silencing on cell adhesion of both MCF-7 and A549 cells.

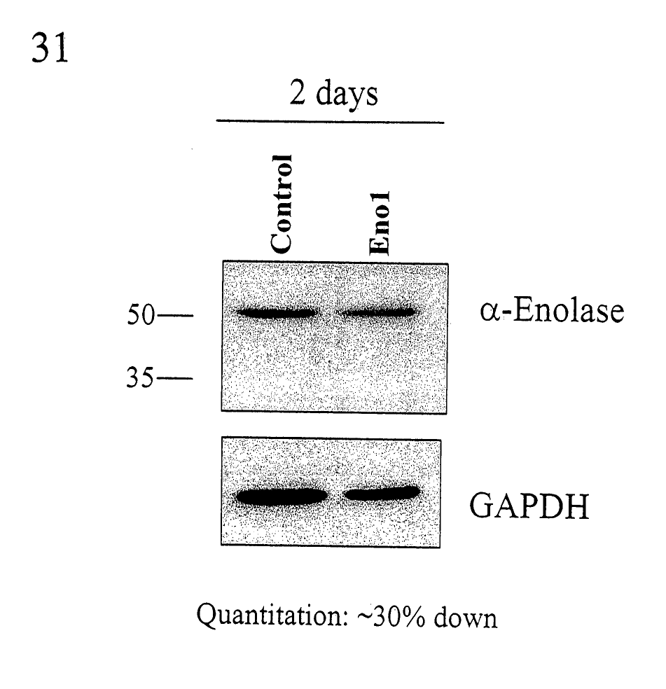

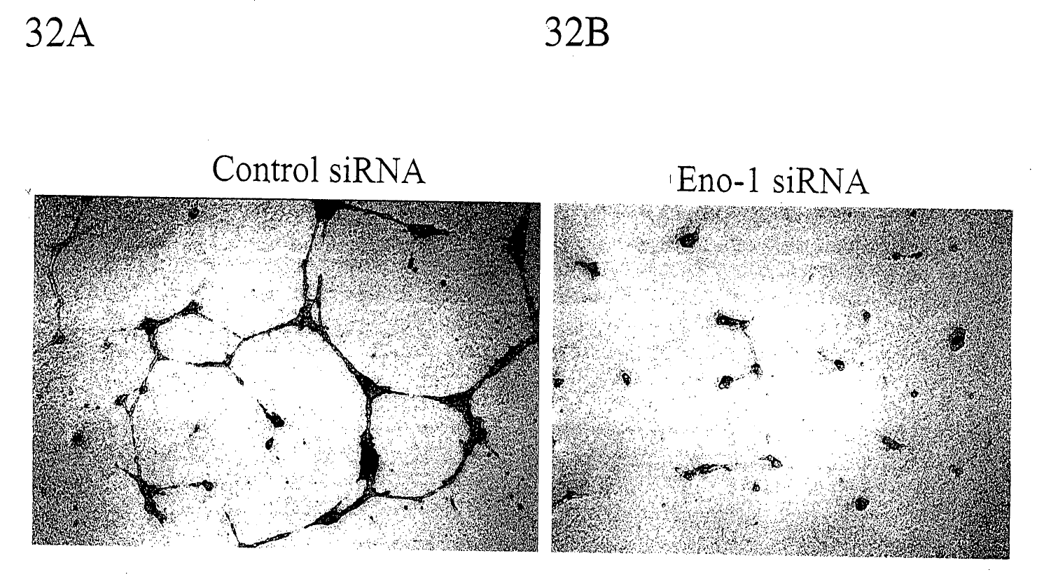

Moreover, the effect of α-enolase expression on metastatic potential was tested using the well-known transwell filter assay (see, e.g., Okada et al. (1996) Arterioscler. Thromb. Vasc. Biol. 16(10):1269-76). These assays test the ability of a cell to move across membranes, which is a hallmark of metastatic potential. The results of transwell filter assays established that down regulation of α-enolase reduced the invasion of A549 cells across membranes as compared to cells with normal levels of α-enolase expression (FIGS. 27A and 27B). Additionally, cells overexpressing α-enolase were more capable of crossing an extracellular membrane than control cells, exhibiting characteristics expected of metastatic cells (FIGS. 27A and 27B). Western blot analysis showed that the levels of expression of α-enolase decreased by 67% at three days, and decreased by 92% at six days after α-enolase silencing was initiated (FIG. 28). In the transwell filter assay, the mock transfected cells and the control siRNA cells continued to exhibit the highly invasive phenotype of the MDA-MB-435 cell line (FIG. 29). However, cells transfected with the α-enolase-targeted siRNA showed a 40% to 50% decrease invasion across a membrane. Modulation of α-enolase expression can affect the invasive nature of cancerous cells.

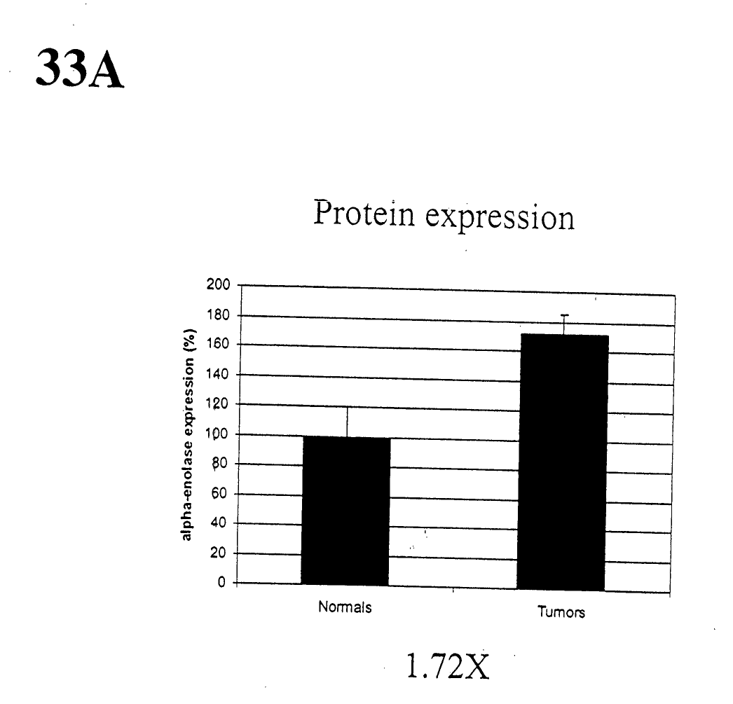

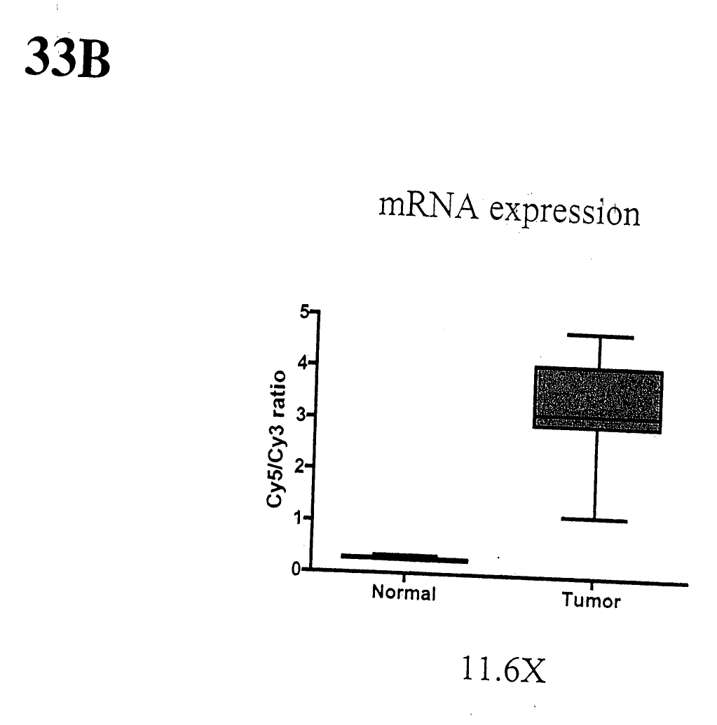

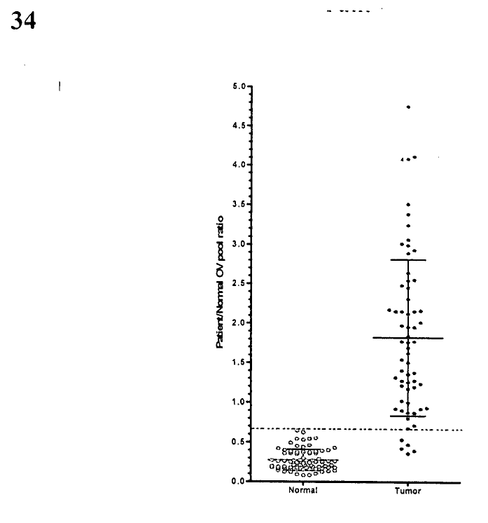

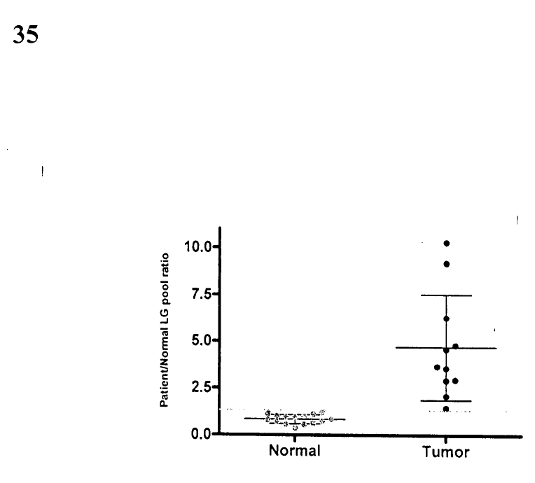

An important characteristic of tumors is their ability to induce vascular growth into the growing tumor to provide nutrients. Angiogenesis has also been linked to increased metastatic potential due to the remodeling of the tumor's vasculature and extracellular matrices (see, e.g., Le et al. (2004) Cancer Metastasis Rev. 23(3-4): 293-310). VEGF has been implicated in the angiogenic phenotype of tumors that have undergone tumor progression. Interestingly, VEGF down regulation by VEGF-directed siRNA decreased the expression α-enolase by nearly 100% in MCF-7 cells (FIG. 30). This result indicates that α-enolase has a role in VEGF-regulated angiogenesis. Because VEGF is a well-known vascularization factor, HUVEC capillary tube formation experiments were performed to determine whether α-enolase affected the angiogenic potential of cells. It is established in the art that formation of capillary tubes is a strong indicator of angiogenic potential in cells. To demonstrate the effect of α-enolase silencing on HUVEC capillary tube formation, siRNA silencing of α-enolase was performed using α-enolase-directed siRNA. The silencing of α-enolase was confirmed by Western Blot analysis (FIG. 31). To demonstrate the effect of α-enolase silencing on angiogenesis, HUVEC capillary tube formation on Matrigel (a basement membrane extract), was monitored in HUVEC cells (FIG. 32). HUVEC cells transfected with control siRNA were able to form capillary tubes on the Matrigel, indicating that the cells maintained the ability to stimulate vascularization (FIG. 32A). However, HUVEC cells transfected with α-enolase targeted siRNA were inhibited from forming capillary tubes on the Matrigel (FIG. 32B). Thus α-enolase silencing inhibits angiogenesis, thereby preventing cells from exhibiting one of the characteristics of tumor progression. FIGS. 33A and 33B showed that α-enolase mRNA and protein levels, respectively, were increased in tumor tissues as compared to normal tissues.

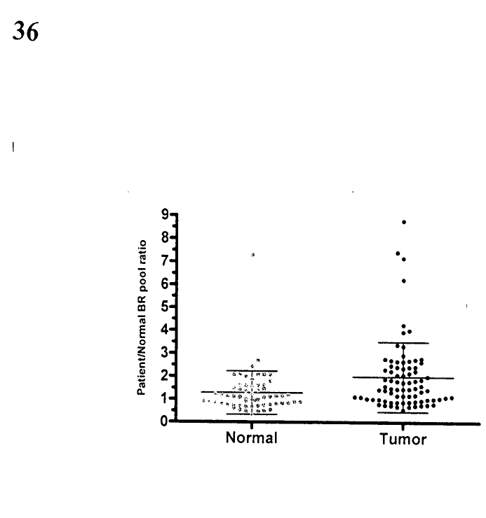

To determine if there was a differential expression of α-enolase in tumor tissue versus normal tissues in vivo, α-enolase mRNA was measured in ovarian, breast and lung tumors obtained from cancer patients at various stages of malignancies and compared to α-enolase mRNA expression in normal, non-cancerous, matched tissues (FIGS. 34-36). The levels of α-enolase mRNA were significantly higher in all three cancer tissues as compared to controls (FIGS. 34-36), suggesting a correlation between α-enolase and malignancy in vivo.

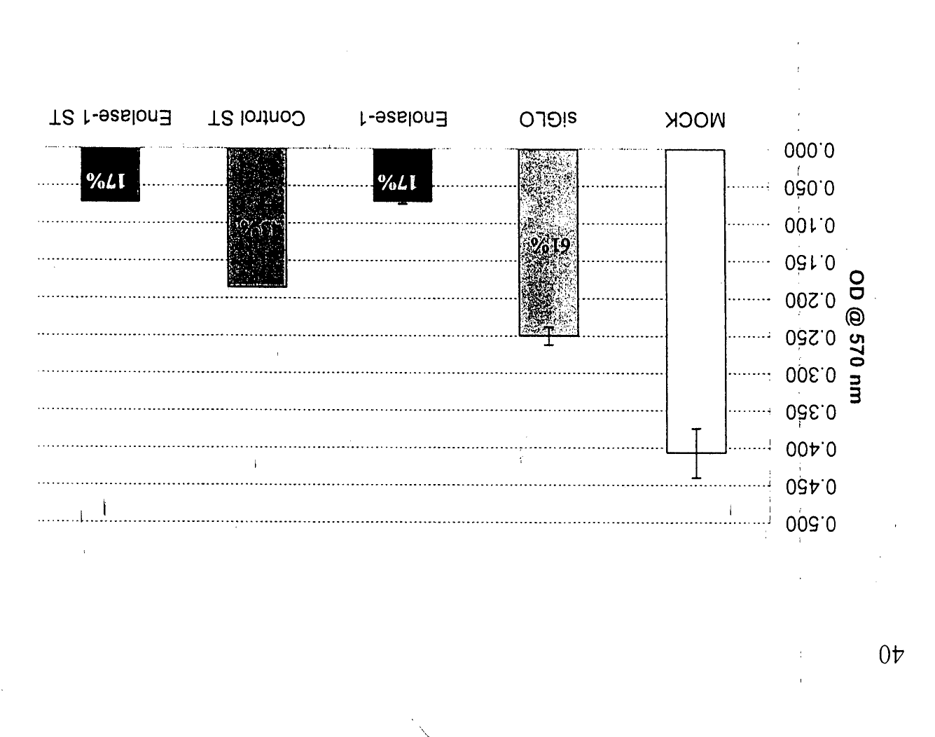

A determinant of cell division and viability, pre-requisites for angiogenesis, was assayed by BrdU incorporation in two breast cancer cell lines. BrdU assays demonstrated that the Eno-1-silenced MCF-7 and MDA-231 breast cancer cells showed a significant (40-50%) decrease in their cell proliferation rates (FIG. 39).

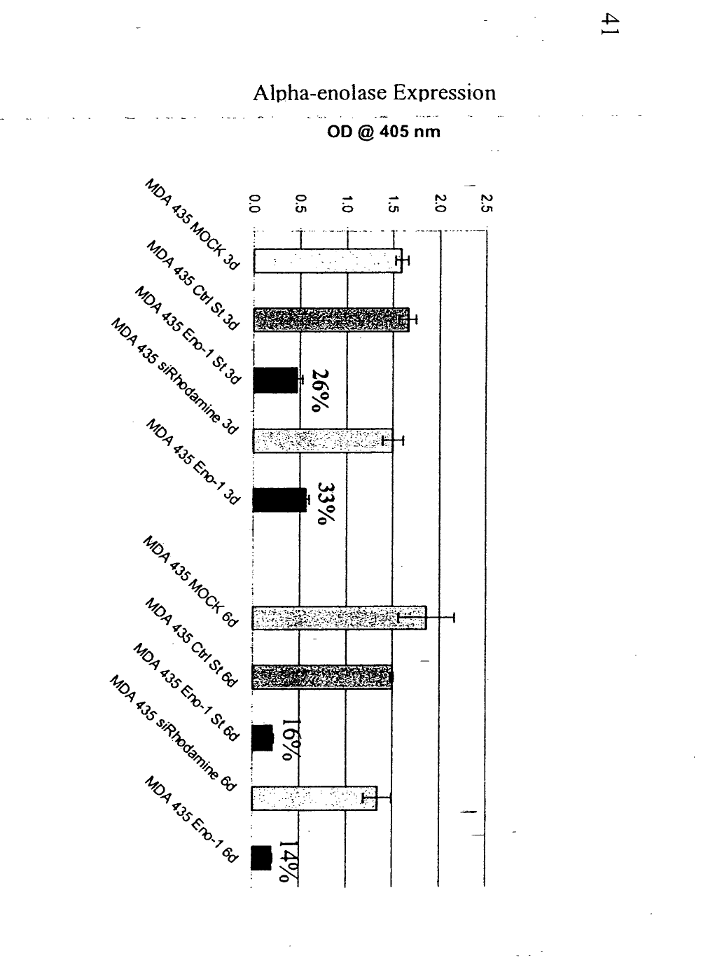

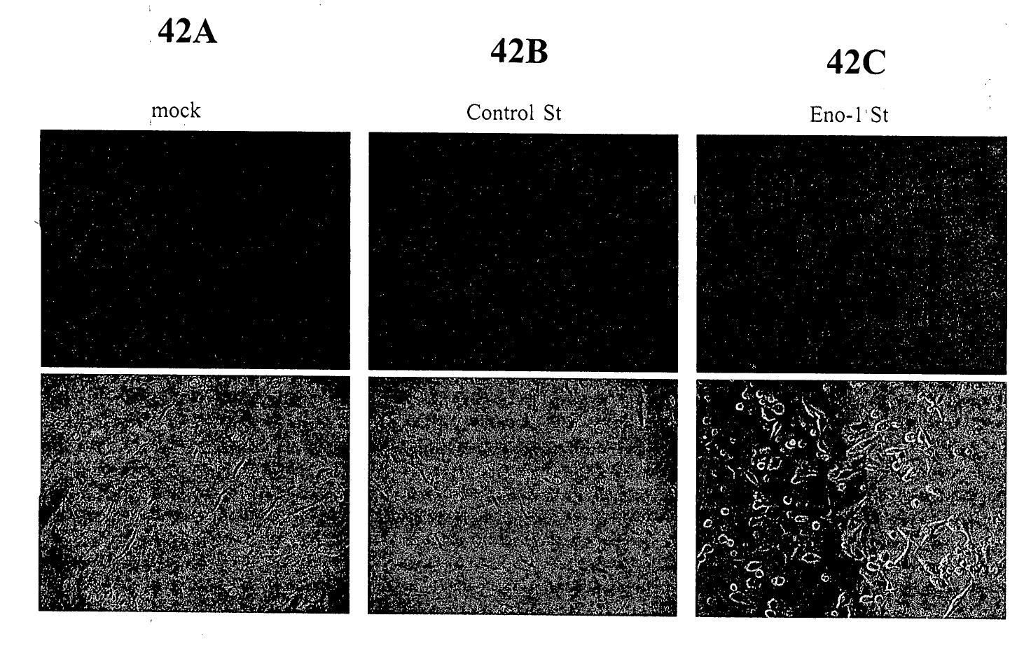

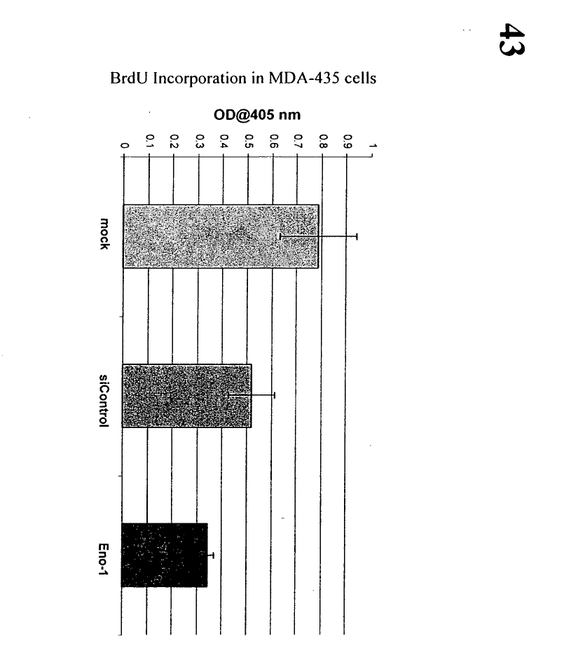

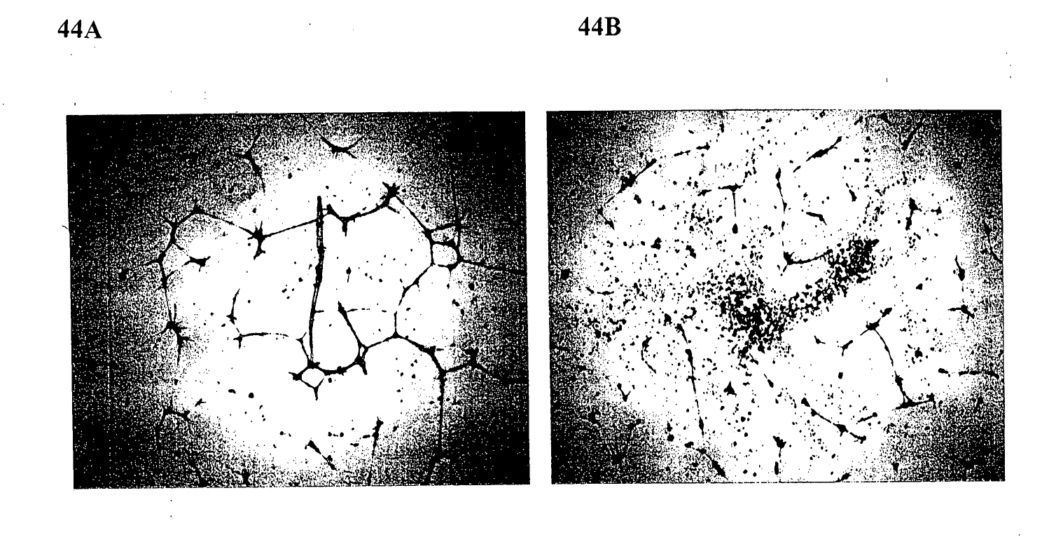

The effect of Eno-1 siRNA silencing on tumor cell growth was even more dramatic in the MDA-435 breast cancer cells, where the viability in cells that were transfected with Eno-1 siRNA dropped to only about 17% of the control level (FIG. 40). In these cells the levels of silencing of α-enolase were also quite high, reaching 14% of the α-enolase levels present in the control transfected cells 6 days post-transfection (FIG. 41). This decrease in cell viability may be due, at least in part, to an increase in apoptosis in the α-enolase-silenced MDA-435 cells as observed by annexin-V FITC staining (FIG. 42). Furthermore, a 50% decrease in DNA synthesis was evident in the α-enolase-silenced cells as shown in FIG. 43. Therefore targeting of the α-enolase protein by RNAi in the MDA-435 cells resulted in a two-punch hit: cell death induction concomitant with a decrease in cell proliferation. Increases in sensitivity to taxol and docetaxel was also observed in the α-enolase-silenced MDA-435 breast cancer cell line.

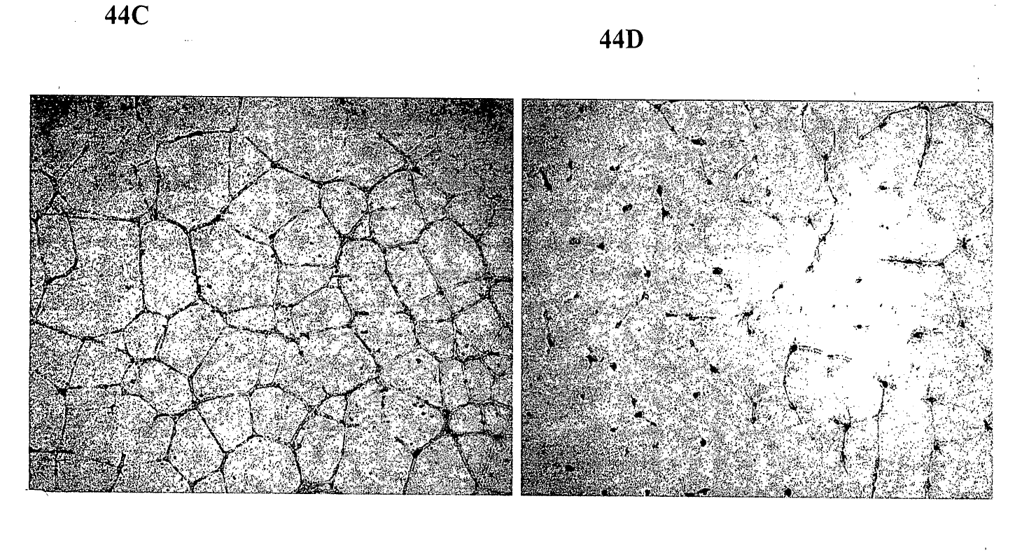

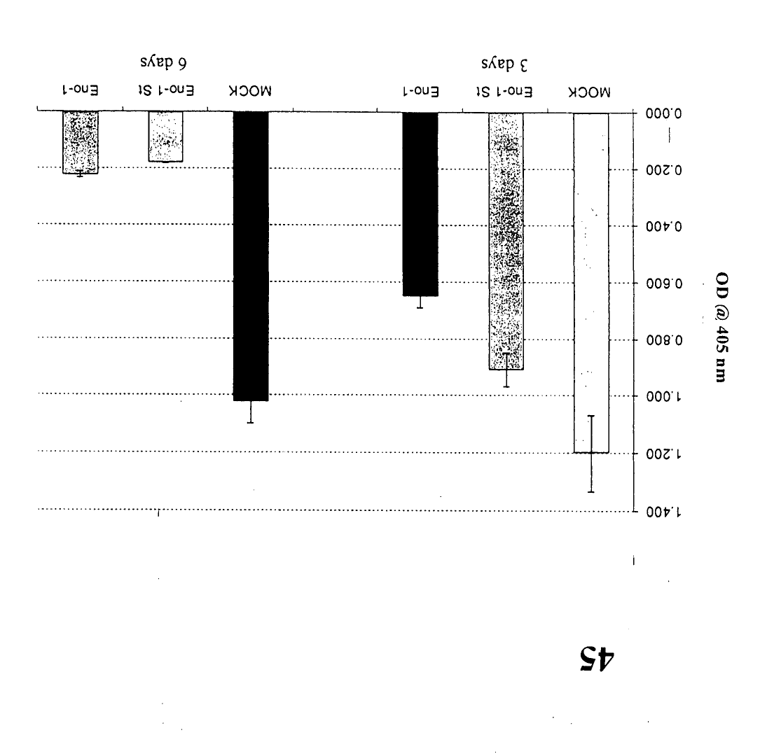

FIGS. 44A-44D show the results of decreasing the levels of expression of enolase in HUVEC cells. HUVEC cells having decreased levels of enolase expression showed far less ability to grow capillary tubes as compared to HUVEC cells transfected with a mock vector (FIGS. 44A-44D). This indicates that enolase expression is involved in the formation of capillary tubes, which is an indicator of angiogenic potential.

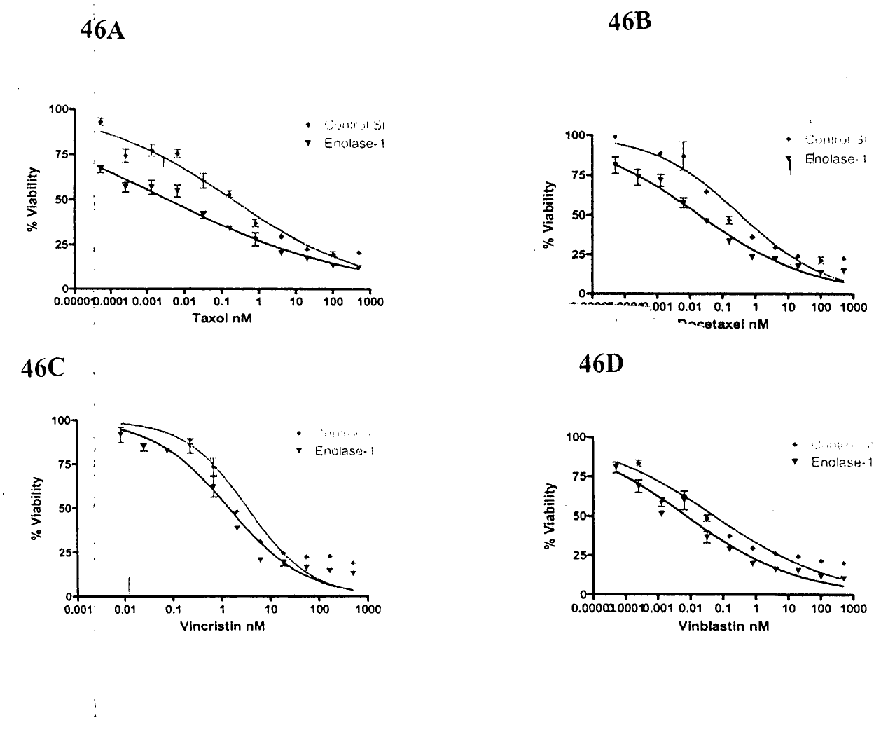

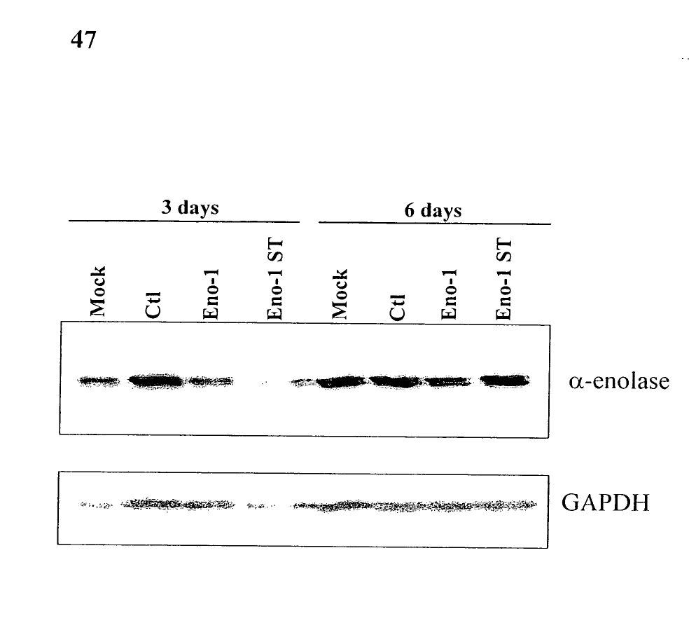

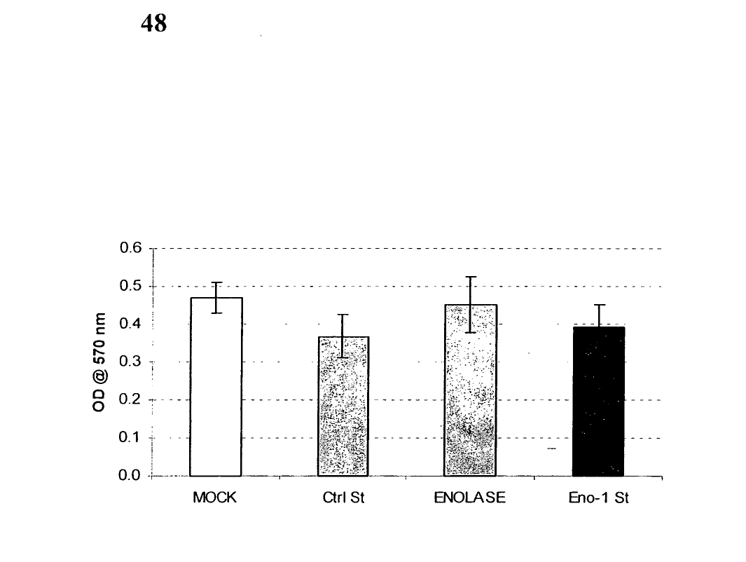

Enolase silencing by siRNA was also successful in the androgen-independent prostate cancer cell line PC-3 (FIG. 45). Targeting this protein in combination with taxol resulted in a 10-fold enhancement in sensitivity (FIG. 46). Enolase siRNA treatment was also synergistic with docetaxel and vinblastine in PC-3 cell killing (FIG. 46). Therefore, α-enolase is a useful target for the treatment of androgen-independent prostate carcinomas.

Overall, the enhanced cytotoxicity observed in α-enolase depleted cells was also observed in multiple tumor cell lines of diverse origins including breast, ovarian, lung, prostate and colon FIGS. 39, 40, 42, 43, and 46).

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

Overexpression of a 44 kD Protein in Cancer Cell Lines

Studies were performed to determine what proteins, if any, were differentially expressed in chemotherapeutic drug-resistant tumor cell lines as compared to their drug-sensitive counterparts. Drug-sensitive cell lines were obtained from were obtained from ATCC (Manassas, Va., USA). MCF7/AR, human lung carcinoma small cell H69, H69/AR, and HL60/AR cells were obtained from McGill University, Montreal, Qc, Canada. MDA-MB-231/AR, MOLT4/AR 250 nM and MOLT4/AR 500 nM cells were derived at Aurelium BioPharma Inc. (Montreal, QC, Canada). Chemotherapeutic drug-resistant cell lines were derived from a drug-sensitive clone of the “parent” cancer cell line representing a particular tissue.

The different cell lines used in the Examples below are listed in Table 1.

TABLE 1

| Drug-Sensitive Cell Lines | Drug-Resistant Cell Lines |
|---|---|
| MCF-7 | MCF-7/AR |
| MDA | MDA-MB231 |
| H69 | H69/AR |
| H460 | H460/AR |
| K562 | K562/AR |
| HSB-2 | HSB-2/AR |
| RPMI-8226 | RPMI-8226/AR |
| MOLT4 | MOLT4/AR |
| HL60 | HL60/AR |

All cell culture materials and reagents were obtained from Gibco Life Technologies (Burlington, Ont., Canada), with the exception of the drugs that were purchased from Sigma Chemical (St. Louis, Mo., USA). Cells were cultured in a MEM medium supplemented with 10% fetal bovine serum (MCF7 and derivatives) or in DMEM high glucose medium supplemented with 10% fetal bovine serum (MDA-MB-231 and derivatives), in RPMI 1640 medium supplemented with 4 mM L-glutamine and 10% fetal bovine serum (H69, H69/AR), in RPMI 1640 medium supplemented with 10 mM HEPES, 1 mM sodium pyruvate, 4.5 g/l glucose and 10% fetal bovine serum (MOLT4 and derivatives), in RPMI 1640 supplemented with 20 mM HEPES, 1 mM sodium pyruvate and 10% fetal bovine serum (K-562) and in RPMI 1640 medium supplemented with 10% fetal bovine serum (HL60 and HL60/AR). All culture media contained L-glutamine (final concentration of 2 mM, except for H69, H69/AR (4 mM)). The cells were grown in the absence of antibiotics at 37° C. in a humid atmosphere of 5% CO2 and 95% air. Chemotherapeutic drug-resistant cells (MCF-7/AR, MDA-MB-231/AR, HL60/AR, MOLT4/AR 250 nM and MOLT4/AR 500 nM) were grown continuously with appropriate concentrations of cytotoxic drugs. All cell lines were examined for and determined to be free of mycoplasma contamination using a PCR-based mycoplasma detection kit according to manufacturer's instructions (Stratagene Inc., San Diego, Calif., USA). All chemotherapeutic drug-resistant cell lines were routinely tested for chemotherapeutic drug resistance using a panel of different drugs representing different classes.

Cell extracts from drug-resistant and drug-sensitive cell lines were prepared to determine the expression levels of potential therapeutic targets in drug-resistant cells. Briefly, cultured cells were rinsed 2 times with 15 ml 1×phosphate buffered saline (“PBS”), and harvested by trypsinization. Cells were collected in a 15 ml tube by centrifugation at 1000 rpm for 5 min. The supernatant was discarded and cells were washed 3 times with 1×PBS. The cell pellet was transferred to an Eppendorf tube and 500 ml of 1×PBS were added. Cells were centrifuged 5 min. at 3000 rpm in an Eppendorf Microfuge. The supernatant was removed and cells were then lysed in 50 ml-150 ml of lysis buffer (50 mM Tris, pH 7.5, 150 mM NaCl, 1% NP-40, 0.5% sodium deoxycholate), containing protease inhibitors (1 mg/ml pepstatin, 1 mg/ml leupeptin, 1 mg/ml benzamidine, 0.2 mM PMSF) and incubated 5 min. on ice. The cell lysates were then centrifuged at 14,000×g for 10 min. at 4° C. The protein concentration of the supernatants was determined by the DC Protein assay (BioRad, Hercules, Calif.). Samples were subsequently stored at −80° C. until ready for analysis.

Total cell lysates were thawed and then incubated with 1 U/ml DNAse 1 (New England BioLabs, Inc., Beverly, Mass.), 5 mM MgCl₂ (final concentration) for 2 hours on ice. Their protein concentration was determined using the RC DC protein assay kit from BIORAD according to manufacturer's instructions (BioRad Laboratories, Hercules, Calif.) (see also Lowry et al., (1951) J. Biol. Chem. 193: 265-275). Lastly, urea was added to the cell lysates to obtain a final concentration of 8 M. Equivalent amounts of proteins (250 mg) from total cell extracts from sensitive (MCF7, MDA-MB-231) and chemotherapeutic drug-resistant cells (MCF7/AR and MDA-MB-231/AR) were analyzed by two-dimensional (2D) gel electrophoresis and visualized by silver staining. This allowed resolution of protein samples according to differences in their isoelectric points in the first dimension and molecular masses in the second dimension. For the first dimension, isoelectric focusing (IEF) was achieved using immobilized pH gradient gel (IPG) strips (pH 4-7, 24 cm, Amersham Pharmacia Biotech, Piscataway, N.J., USA). Briefly, 24 cm strips were rehydrated in a ceramic strip holder in 450 ml rehydration buffer (8 M urea, 2% (w/v) CHAPS, 0.5% (v/v) IPG buffer and 0.0125% bromophenol blue) containing the protein samples for 15 hours at 30 volts. Electrode pads were then placed over each electrode and the proteins separated on an IPGphor unit using the following program: 24 cm strips (pH 4-7) at −500 V for 500 Vh, −1000 V for 1000 Vh, and −8000 V for 32000 Vh. Upon completion of IEF, strips were then slightly rinsed with water and equilibrated in 1% DTT in equilibration buffer (50 mM Tris/HCl, pH 8.8, 6 M urea, 30% glycerol, 2% (w/v) SDS and 0.0125% bromophenol blue) for 15 min, followed by 4% iodoacetamide in equilibration buffer for 15 min.

For the second dimension, the above isoelectric strips were subject to sodium dodecyl sulfate-polyacrylamide gel electrophoresis (SDS-PAGE) using a 12.5% gel, according to the method of Laemmli (Laemmli (1970) Nature 227:680-685). Molecular weight markers were loaded onto a 2×3 mm filter paper and placed at one end of the strip. The strip and molecular weight marker filter were then sealed onto the polyacrylamide gel with a 0.5% agarose solution in running buffer. The gels were run at constant current (5 mA/gel) for 30 min., and then the current was increased to reach 10 mA/gel for 6 hours.

Two-dimensional gels were fixed in 40% (v/v) methanol, 10% (v/v) acid acetic solution for 24 h at room temperature and then silver stained. Briefly, gels were incubated in 750 ml of a sensitizing solution (30% EtOH, 10 mM potassium tetrathionate, 500 mM potassium acetate in nanopure H₂O) for 40 min., then washed 6 times with 750 ml of nanopure H₂O, incubated 30 min. in 750 ml of a staining solution (12.5 mM silver nitrate in nanopure H₂O), washed again for 15 seconds in 750 ml of nanopure H₂O, and developed in 750 ml of developing solution (250 mM potassium carbonate, 0.00125% (w/v) sodium thiosulfate, 0.01% formaldehyde in nanopure H₂O). The development of the gels was stopped when the desired intensity of staining was reached by transferring the gels in the stopping solution (300 mM Tris, 2% acetic acid in nanopure H₂O). The 2D maps of total cell extracts were compared by using ImageMaster 2D Elite software (Amercham Pharmacia Biotech) and checked manually (FIG. 1).

### Example 2

### Identification of a 44 kD Protein in MCF-7/AR Breast Cancer Cells as α-Enolase

To discover the identity of the 44 kD protein that was overexpressed in drug-resistant cell lines, the spot located on the 2D gel was subjected to mass spectrometry. FIGS. 1A and 1B show a 44 kD spot to be up-regulated by 3.4 times in the drug-resistant cell samples. The 44 kD spot was isolated and subjected to tryptic digestion in preparation for mass spectrometry. The spot of interest was excised with a clean (clean; acid washed) razor blade and cut into small pieces on a clean glass plate and transfer into a 200 μl PCR tube (MeOH treated). The gel pieces were mixed with 50 μl destainer A and 50 μl destainer B (provided with SilverQuest kit, Life Technologies) (or 100 μl of the destainers premix prepared fresh) and incubated for 15 min. at room temperature (RT) without agitation. The destaining solution was removed using a capillary tip. Water was added to the gel pieces, mix and incubate 10 min at RT. The latter step was repeated three times. The gel pieces were then dehydrated in 100 μl 100% methanol for 5 min. at RT, followed by rehydration in 30% methanol/water for 5 min. Gel pieces were then washed 2 times in water for 10 min. and 2 times in 25 mM Ambic (ammonium bicarbonate), 30% (v/v) acetonitrile for 10 min.

After complete drying in a speed vac for 20 min., tryptic digestion of the destained and washed gel pieces was performed by adding about 1 volume of trypsin solution (130 ng of trypsin (Roche Diagnostics, Laval, Qc, Canada) in 25 mM ammonium bicarbonate, 5 mM CaCl2) to 1 volume of gel pieces and samples left on ice for 45 min. Fresh digestion buffer was added and digestion allowed to proceed for 15-16 hrs at 37° C. Digested peptides were extracted with acetonitrile for 15 min. at RT with shaking. The gel pieces/solvent were sonicated 5 min. and re-extracted with 5% formic acid: 50% acetonitrile:45% water freshly prepared. The extraction step was repeated several times and the collected material combined and lyophilized to dryness. The extracted peptides were resuspended in 5% methanol with 0.2% trifluroacetic acid then loaded on an equilibrated C18 bed (Ziptip from Millipore, Bedford, Mass., USA). The loaded Ziptip was washed with 5% acetonitrile containing 0.2% TFA and then eluted in 10 ml of 60% acetonitrile. Eluted peptide solution was dried and analyzed using MALDI mass spectroscopy (Mann et al. (2001) Ann. Rev. Biochem. 70: 437-473). The resulting peptides list was further analyzed using the sequence database search shareware software program ProFound™ (http://www.proteomics.com/prowl-cgi/Profound.exe) to obtain protein identity. PROFOUND was used to search public databases for protein sequences (e.g., non-redundant collection of sequences at the US National Center for Biotechnology Information (NCBInr)). The NCBInr database contains translated protein sequences from the entire collection of DNA sequences kept at Genbank, and also the protein sequences in the PDB, SWISS-PROT and PIR databases. Eleven peptide fragments were analyzed, and showed 100% homology to the human α-enolase sequence (FIG. 2). According to these results, the 44 kD spot is the α-enolase protein.

### Example 3

### Identification of α-Enolase Overexpression in MOLT4/AR Cell Lines

Western blot analysis utilizing anti-α-enolase antibodies was performed on MOLT4 cell extracts fractionated on SDS-PAGE (FIG. 3). Cells extract were prepared according to the protocol detailed in Example 1. Total cell lysates were thawed, then 100 mg of protein (completed to 50 ml with lysis buffer) were mixed with 10 ml of 5× electrophoresis buffer (60 mM Tris-HCl, pH 6.8, 25% glycerol, 2% SDS, 14.4 mM β-mercaptoethanol, 0.1% bromophenol blue) and these samples were heated at 100° C. for 5 min. and loaded on 10% SDS-PAGE gels. Resolved proteins were electrophoretically transferred onto nitrocellulose membranes (Hybond, Amercham Pharmacia Biotech) for 1 hour. FIG. 3D shows the overexpression of α-enolase in Molt-4 cells selected with adriamycin.

After blocking the membranes with 5% non-fat milk in 1×PBS overnight at 4° C., all antibody-binding reactions were performed in 5% non-fat milk in 1×PBS for 2 hours at room temperature for primary antibodies and for 1 hour for secondary antibodies coupled to HRP. The signal was detected by the Supersignal west Pico chemiluminescent substrate (Pierce, Rockford, Ill., USA). Two polyclonal antibodies against α-enolase were used: a goat antibody against human α-enolase (Santa Cruz Biotechology, Inc., Santa Cruz, Calif.); rabbit polyclonal antibody against human α-enolase (NNE) (MorphoSys AG, Munich, Del.). Membranes were subsequently blotted with anti-GAPDH (Novus Biologicals, Inc., Littleton, Colo.) or anti-prohibitin mouse monoclonal antibody (Lab Vision Corp., Fremont, Calif.) as controls for protein loading.

### Example 4

### Identification of α-Enolase Overexpression in Other Drug-Resistant Cell Lines

To determine if α-enolase was overexpressed in MCF7/AR, MDA/AR, HL60/AR cell lines compared to cell lines isolated from the same tissue type, Western blot analysis was performed on the cell lines as described in Example 3. Alpha-enolase was strongly induced in adriamycin-resistant breast cancer cell lines MCF-7 and MDA-MB231 (FIG. 3A). FIGS. 3B-3D show the positive induction of the levels of expression of α-enolase in SKOV3, H69, K562, HSB-2, RPMI-8226, HL60, Molt-4, and H460 adriamycin-resistant cells. In addition, elevated levels of α-enolase mRNA and protein were found in ovarian tumor tissues as compared to normal tissues (FIGS. 33A and 33B). These results indicate that the resistant phenotype of several diverse cancer cells lines correlates with an overexpression or induced-expression of α-enolase both in mRNA translation and protein production.

### Example 5

### Targeted Silencing of α-Enolase in Cell Lines

To establish the importance of α-enolase to the expression of the drug-resistant phenotype in cell lines, α-enolase expression was silenced using RNAi. Briefly, the following siRNA duplexes targeting the human α-enolase mRNA were designed and purchased either from Ambion (Austin, Tex.) or Invitrogen (Carlsbad, Calif.). The siRNA duplex sequences were: sense strand 5′-GGCUGUUGAGCACAUCAAUtt-3′ (SEQ ID NO: 1); antisense strand: 5′-AUUGAUGUGCUCAACAGCCtt-3′ (SEQ ID NO: 2) targeting the mRNA sequence corresponding to nucleotides 343-352 from the start of the transcript (Ref Seq ID number: NM_{—}001428). This siRNA duplex was predesigned, synthesized with 3′TT overhangs, purified and annealed by Ambion (Austin, Tex.) (Table 2).

TABLE 2
Small Interfering RNA Duplexes Targeting α-Enolase

| | | |
|---|---|---|
| siRNA Duplex | Sequence | SEQ. ID NO: |

| | | |
|---|---|---|
| Enolase siRNA (Sense) | 5′-GGCUGUUGAGACAUCAAU-3′ | 1 |
| Enolase siRNA (Anti-sense) | 5′-AUUGAUGUGCUCAACAGCC-3′ | 2 |
| Eno-1 Stealth siRNA (Sense) | 5′-CUCAAAGGCUGUUGAGACAUCAAU-3′ | 3 |
| Eno-1 Stealth siRNA (Anti-sense) | 5′-AUUGAUGUGCUCAACAGCCUUUGAC-3′ | 4 |
| Eno-2 Stealth siRNA (Sense) | 5′-CCAGUGGUGCUUCAACUGGUAUCUA-3′ | 5 |
| Eno-2 Stealth siRNA (Anti-sense) | 5′-UUCUUUGGUCUGCAUUCACAUUUGU-3′ | 6 |
| Scrambled Mock Sequence | 5′-CCAGGGUUCCUAAUCGGAUUUGCUA-3′ | 7 |
| VEGF Stealth siRNA (Sense) | 5′-ACAAAUGUGAAUGCAGACCAAAGAA-3′ | 8 |
| VEGF Stealth siRNA (Anti-Sense) | 5′-UUCUUUGGUCUGCAUUCACAUUUGU-3′ | 9 |

Two chemically modified Stealth™ RNAi duplexes targeting α-enolase were designed using the Block-it™ RNAi Designer tool by Invitrogen (http://rnaidesigner.invitrogen.com/sima/design.do). The corresponding duplexes were: ENO-1 sense strand 5′-CUCAAAGGCUGUUGAGCACAUCAAU-3′ (SEQ ID NO: 3) and antisense strand 5′-AUUGAUGUGCUCAACAGCCUUUGAC-3′ (SEQ ID NO: 4) targeting nucleotides 337-352 of the α-enolase mRNA. ENO-2 siRNA duplex: sense strand 5′-CCAGUGGUGCUUCAACUGGUAUCUA-3′ (SEQ ID NO: 5) targeting the sequence corresponding to nt. 258-283 from RefSeq NM_{—}001428. As a negative control, the scrambled sequence 5′-CCAGGGUUCCUAAUCGGAUUUGCUA-3′ (SEQ ID NO: 7) without significant homology to any human gene was designed. The siRNA duplex targeting VEGF was the chemically modified Stealth version of the following duplexes: sense strand 5′-ACAAAUGUGAAUGCAGACCAAAGAA-3′ (SEQ ID NO: 8); antisense strand 5′-UUCUUUGGUCUGCAUUCACAUUUGU-3′ (SEQ ID NO: 9) (Filleur et al. (2003) Cancer Res. 63(14):3919-22). All above duplexes were synthesized, purified and annealed by the manufacturer (Invitrogen). To monitor transfection efficiency, a Cy3 labeled GL2 siRNA duplex against firefly luciferase was purchased from Dharmacon, Inc. (Chicago, Ill.). For the chemically modified Stealth siRNAs the non-targeting siGLO™ fluorescent siRNA duplex (Dharmacon, Chicago, Ill.) or the Block-it™ Fluorescent oligonulceotide (Invitrogen, Carlsbad, Calif.) were used. Transfection efficiencies were typically evaluated 24-48 hrs post transfection using a fluorescence microscope. The levels achieved were routinely greater than 95%.

For a typical siRNA transfection, 1 nmole of the annealed siRNA duplex was mixed with 1.4 ml of Opti-MEM reagent (Invitrogen). In another tube, 85 ml of Oligofectamine reagent (Invitrogen, Carlsbad, Calif.) is mixed with 600 ml of Opti-MEM. The two solutions are combined and mixed gently by inversion and incubated for 20 min. at RT. The resulting solution is added to the cultured cells drop by drop in a 10 cm dish (cells are approximately 40-50% confluent). The next day the transfected cells were trypsinized and seeded in 6 or 96-well plates and further incubated for the indicated amount of time (assay dependent) before further analysis.

To determine the ability of cells to proliferate after transfection with a vector containing the coding sequences for the α-enolase gene or control siRNAs as indicated above, the cells were seeded the next day in a 96-well plate at 5×10³ cells/well in quadruplicate. The plate was incubated at 37° C. incubator for another 72 hrs. The media was removed, and 100 ml of CyQUANT GR dye/cell lysis buffer (Molecular Probes, Inc., Eugene, Oreg.) was added per well. The plate was incubated for 5 min. at RT in the dark. The resulting fluorescence was measured in a Wallac microplate reader (PerkinElmer, Inc., Boston, Mass.) using a 535 nm filter. Results were the average of quadruplicates and were plotted in Excel. The number of cells was determined by extrapolation from a standard curve.

In order to further investigate the potential involvement of α-enolase in drug resistance, RNAi technology was employed to silence α-enolase expression in breast and ovarian cancer cells. Initially, a pre-designed siRNA duplex targeting human α-enolase (Ambion, Inc., Austin, Tex.) was employed (Table 1). Two additional chemically modified Stealth siRNA duplexes were designed using RNAi designer resources (Invitrogen Corp., Carlsbad, Calif.) (Table 1).

Alpha-enolase directed siRNA silencing was performed in the human non-small cell lung carcinoma cell line A549 (FIG. 12). Western blot experiments were performed to show the levels of expression of α-enolase in cells treated with an α-enolase-targeted siRNA and cells treated with a control siRNA (FIG. 15). MTT assays were performed to establish the effects of α-enolase-targeted siRNA on H460 cell viability (FIG. 16).

### Example 6

### Effects of α-Enolase Silencing on Cell Survival

Cell survival was determined using the MTT cytotoxicity assay (see, e.g., Tokuyama et al. (2005) Anticancer Res. 25(1A): 17-22). Small interfering RNA transfected cells were seeded in triplicate into 96-well plates at 5×10³ cells/well 48 hrs post-transfection. The cells were incubated for an additional 16-24 hrs before they were exposed to increasing concentrations of cytotoxic drugs. Doxorubicin (adriamycin), cisplatinum, etoposide, docetaxel, taxol, vinblastin, vincristin, melphalan, mitoxantrone, and thiotepa were all purchased from Sigma Corp. (St. Louis, Mo.). Stocks were made as follows: 6 mM for doxorubicin, 1.1 mM for vincristin and vinblastin, 500 mM for thiotepa (all in sterile H₂O); 1.1 mM for taxol, 50 mM for cisplatinum both in DMSO; and 137 mM for melphalan, 0.97 mM mitoxantrone in ethanol. Appropriate dilutions were made in the respective media for each cell line. Following addition of drugs, incubation was continued for an additional 72 hrs. Twenty-five ml of MTT dye (5 mg/ml) were added into each well and the plate was further incubated at 37° C. for 4 hrs. The dye was solubilized with 10% Triton X-100, 0.01 N HCl and further incubated at 37° C. in the dark for 30 min. Cell viability was determined by measure of absorption at 570 nm in a Wallac multiwell plate reader (PerkinElmer, Inc., Boston, Mass.). The averages of triplicate wells were plotted using the Prism software (GraphPad Software, Inc., San Diego, Calif.).

Utilizing a clonogenic assay generated additional information concerning cell viability after drug-resistant cells were exposed to siRNA and chemotherapeutics. Briefly, transfected MCF-7 cells were seeded in triplicate into 24-well plates at 5×10³ cells/well 48 hours post-transfection. The cells were incubated for an additional 16-24 hrs before they were exposed to increasing concentrations of cytotoxic drugs. Taxol or Vincristine was added at IC₁₀ or IC₅₀ concentrations determined from MTT experiments for MCF-7 cells. The IC₁₀ for taxol was 1 nM and IC₅₀=100 nM; for vincristin, IC₁₀ and IC₅₀ were determined to be 5 pM and 0.25 nM, respectively. The cells were further incubated for an additional 7 days. At the end of the incubation, the cells were stained with addition of a 0.5% Methylene Blue solution in 50% ethanol for 15 min. at RT. The staining solution was then removed and plates were dried overnight. The plates were scanned and the stained colonies were solubilized in 0.1% SDS. The absorbance of the resulting solution was determined by spectrophotometry at 660 nm. Results were plotted as bar graphs using Excel as shown in FIGS. 10A and 10B. Thus, the enhanced taxol cytotoxicity observed in α-enolase depleted cells was confirmed with two different assays in multiple cell lines, arguing for an α-enolase-mediated cell survival mechanism in response to taxanes and vinca alkaloids.

To evaluate the effect of α-enolase depletion on the chemosensitivity of cells to cytotoxic agents, MTT assays were performed in breast adenocarcinoma MCF-7 cells transfected with α-enolase specific siRNAs. The α-enolase silenced MCF-7 cells displayed a substantial increase in their sensitivity to taxol and vincristin as evidenced by the cytotoxicity graphs. The results are shown in FIGS. 8C and 8D. The predicted EC₅₀ values (the effective dose of a compound responsible for a positive effect half-way between baseline and maximum net impact) for the α-enolase-depleted cells are shown in Table 3. Similarly, results using other chemotherapeutic drugs are also shown in the table indicating the sensitivity of breast cancer tissue to α-enolase depletion.

TABLE 3
Effects of Alpha-Enolase Specific siRNA on the Sensitivity of
MCF-7 Tumor Cells to Drugs

| Chemotherapeutic Drug | Control (EC₅₀) | α-Enolase (EC₅₀) |
|---|---|---|
| Doxorubicin (μM) | 90.64 (R2 = 0.9665) | 62.65 (R2 = 0.9562) |
| | | 1.4 × IS |
| Taxol (nM) | 78.4 (R2 = 0.8825) | 7.157 (R2 = 0.9252) |
| | | 10.9 × IS |
| Cisplatinum (μM) | 117.7 (R2 = 0.9815) | 92.03 (R2 = 0.9859) |
| | | 1.3 × IS |
| Etoposide (μM) | 16.11 (R2 = 0.9443) | 27.05 (R2 = 0.941) |
| | | 1.6 × IR |
| Vincristine (μM) | 163.3 (R2 = 0.8647) | 12.64 (R2 = 0.8847) |
| | | 12.9 × IS |
| Mitoxantrone (μM) | 8.682 (R2 = 0.9616) | 7.817 (R2 = 0.946) |
| | | 1.1 × IS |
| Docetaxel (nM) | 73.49 (R2 = 0.9195) | 36.45 (R2 = 0.9259) |
| | | 2.0 × IS |
| Melphalan (μM) | 14.7 (R2 = 0.9705) | 11.5 (R2 = 0.9631) |
| | | 1.3 × IS |

IS: “increased sensitivity” to the particular drug.
IR: “increased resistance” to the particular drug.
R2: the fit of all experimental data on a curve, representing the statistical value of the data. As R2 approaches 1, the fit to the curve becomes increasingly improved.

These observations were expanded by targeting α-enolase in the ovarian cancer cell line, CaOV3. Notably, α-enolase-directed siRNA decreased chemotherapeutic drug resistance by 6-8 fold for vincristine and vinblastine respectively, and by 24.6 fold for taxol, when compared to mock siRNA CaOV3 transfected cells (see Table 4).

TABLE 4
Transfection of CaOV3 Cells with α-Enolase Specific RNAi

| Chemotherapeutic | | |
|---|---|---|
| Drug | Control (EC₅₀) | α-Enolase (EC₅₀) |
| Taxol (nM) | 2.69 (R2 = 0.97) | 0.1096 (R2 = 0.96) |
| | | 24.6 × IS |
| Vincristine (nM) | 0.08403 (R2 = 0.7853) | 0.01082 (R2 = 0.8354) |
| | | 7.8 × IS |
| Vinblastin (nM) | 0.02198 (R2 = 0.9201) | 0.003491 (R2 = 0.9276) |
| | | 6.3 × IS |

IS: “increased sensitivity” to the particular drug.
R2: the fit of all experimental data on a curve, representing the statistical value of the data. As R2 approaches 1, the fit to the curve becomes increasingly improved.

The EC₅₀ results in this experiment were obtained 72 hours post mock-transfection or transfection with either irrelevant, or α-enolase RNAi. These data expand the observations seen in Table 3 to include the ovarian cancer cell line, CaCOV3, and demonstrate the reduction of α-enolase expression in resistant cell lines renders them more sensitive to pharmacological effects of chemotherapeutic agents.

Cell survival was deduced in α-enolase silenced A549 cells treated with chemotherapeutic drugs including doxorubicin, cisplatinum, taxxol, etoposide, mitoxandrone, docetaxel, vincristin and vinblasin (FIGS. 13A-13H). The results are summarized in Table 5.

TABLE 5
Effects of Alpha-Enolase Specific siRNA on the
Sensitivity of A549 Tumor Cells to Drugs

| Chemotherapeutic Drug | Control (EC₅₀) | α-Enolase (EC₅₀) |
|---|---|---|
| Doxorubicin (μM) | 0.2891 (R2 = 0.9351) | 0.2969 (R2 = 0.9496) |
| | | 1.0 × IR |
| Cisplatinum (μM) | 326.8 (R2 = 0.9431) | 218.4 (R2 = 0.9641) |
| | | 1.5 × IS |
| Taxol (nM) | 5.944 (R2 = 0.9646) | 3.158 (R2 = 0.9659) |
| | | 1.9 × IS |
| Etoposide (μM) | 20.47 (R2 = 0.9252) | 10.11 (R2 = 0.9661) |
| | | 2.0 × IS |
| Mitoxandrone (nM) | 136.2 (R2 = 0.9483) | 88.84 (R2 = 9597) |
| | | 1.5 × IS |
| Docetaxel (nM) | 7.014 (R2 = 0.9419) | 0.3507 (R2 = 9664) |
| | | 20 × IS |
| Vincristin (nM) | 83.31 (R2 = 0.9077) | 19.99 (R2 = 0.9651) |
| | | 4.2 × IS |
| Vinblastin (nM) | 2.394 (R2 = 0.9224) | 0.07604 (R2 = 0.9152) |
| | | 31.5 × IS |

IS: “increased sensitivity” to the particular drug.
IR: “increased resistance” to the particular drug.
R2: the fit of all experimental data on a curve, representing the statistical value of the data. As R2 approaches 1, the fit to the curve becomes increasingly improved.

To investigate if α-enolase is broadly involved in mediating drug resistance to microtubule targeting agents, non-small cell lung cancer cell lines A549 and H460, respectively, were transfected with α-enolase siRNA and enolase expression and viability were assessed. Enolase-1 siRNA's were effective in down-regulating α-enolase expression in A549 cells (FIG. 23) and caused a decrease in cell viability. Alpha-enolase-silenced A549 cells showed significant shifts in their chemosensitivities to docetaxel, taxol, vinblastine and vincristin as (FIGS. 24A and 24B). The biggest effect was obtained with docetaxel-treated A549 cells where a 12-15 fold shift in the EC₅₀ values (obtained 72 hours post transfection) was observed in the α-enolase silenced cells as compared to the control siRNA treated cells (Table 5).

Apoptotic cells were measured following incubation with siRNAs. This assay was performed to determine the number of cells that were now susceptible to chemotherapeutic drugs. Cells transfected with siRNA were seeded in Lab-Tek 16-well chamber slides (Electron Microscopy Sciences, Hatfield, Pa.) at 10⁴ cells/well 48 hrs post-transfection. Apoptosis was determined 16 hours later by annexin-V staining using the Annexin-V FLUOS kit (Roche, Ltd., Basel, CH) following the manufacturer's instructions. Slides were observed under a fluorescence microscope and images were taken using an Olympus digital camera and the Q-Capture software (QImaging, Burnaby, BC, CA).

There was a substantial amount of Annexin-V-positive cells in the α-enolase siRNA transfected MCF-7 cells as compared to cells treated with a control siRNA (FIGS. 7C and 7D). Similar effects on cell morphology and viability were observed with all three enolase-specific siRNA duplexes but not with control siRNAs (FIG. 6).

To establish the effectiveness of α-enolase silencing on other cancer cell types, H460 non-small cell lung cancer cells were challenged with a chemotherapeutic drug, MTT cytotoxicity assays were carried out and survival curves plotted as shown in FIG. 17. Similar effects were observed; α-enolase silencing resulted in a 2-4 fold enhancement of the efficacy observed with all the drugs used (Table 6).

TABLE 6
Effects of Alpha-Enolase Specific siRNA on the
Sensitivity of H460 Tumor Cells to Drugs

| Chemotherapeutic Drug | Control (EC₅₀) | α-Enolase (EC₅₀) |
|---|---|---|
| Doxorubicin (nM) | 118.9 (R2 = 0.9584) | 29.96 (R2 = 0.9597) |
| | | 4.0 × IS |
| Cisplatinum (nM) | 44.42 (R2 = 0.9731) | 24.04 (R2 = 0.9756) |
| | | 1.8 × IS |
| Taxol (nM) | 2.236 (R2 = 0.966) | 0.6019 (R2 = 0.9941) |
| | | 3.7 × IS |
| Etoposide (μM) | 5.367 (R2 = 9658) | 1.912 (R2 = 0.9745) |
| | | 2.8 × IS |
| Mitoxantrone (nM) | 223.6 (R2 = 0.9067) | 70.79 (R2 = 0.9445) |
| | | 3.2 × IS |
| Docetaxel (nM) | 1.365 (R2 = 0.9311) | 0.663 (R2 = 0.9967) |
| | | 2.2 × IS |
| Vincristin (nM) | 4.964 (R2 = 0.9127) | 2.026 (R2 = 0.9848) |
| | | 2.5 × IS |
| Vinblastin (nM) | 3.09 (R2 = 0.9284) | 1.539 (R2 = 0.9766) |
| | | 2.0 × IS |

IS: “increased sensitivity” to the particular drug.
R2: the fit of all experimental data on a curve, representing the statistical value of the data. As R2 approaches 1, the fit to the curve becomes increasingly improved.

The colorectal adenocarcinoma cell line SW-480 was treated with α-enolase-directed siRNA using the procedures described above. Eno-1 siRNA was quite effective in silencing α-enolase expression in these cells as shown in FIG. 18. siRNA treatment of SW-480 cells resulted in a 50% decrease in cell viability. The α-enolase-depleted cells also showed greatly enhanced chemosensitivity to taxol and vincristine (FIGS. 19A and 19B and Table 7).

TABLE 7
Effects of Alpha-Enolase Specific siRNA on the
Sensitivity of SW-480 Tumor Cells to Drugs

| Chemotherapeutic Drug | Control (EC₅₀) | α-Enolase (EC₅₀) |
|---|---|---|
| Taxol nM | 0.276 | 0.03295 |
| | R2 = 0.4605 | 8.4 × IS |
| | | R2 = 0.6759 |
| Vincristin uM | 0.04583 | 0.000221 |
| | R2 = 0.7564 | 207.4 × IS |
| | | R2 = 0.6517 |

IS: “increased sensitivity” to the particular drug.
R2: the fit of all experimental data on a curve, representing the statistical value of the data. As R2 approaches 1, the fit to the curve becomes increasingly improved.

The above results indicate that cells treated with α-enolase targeted siRNA show decreased expression levels of α-enolase. It is apparent that α-enolase silencing decreases the viability and chemotherapeutic drug resistance of cells. In some cases, chemosensitivity increased by 1.5 to approximately 70 times that of the untreated cells of the same type.

Similar tests utilizing α-enolase siRNA were performed on MDA-435 breast adenocarcinoma cells. Chemosensitivity to various drugs was determined for the MDA-435 cells as described above. The impact on viability of MDA-435 cells following Eno-1 siRNA transfection is indicated in FIGS. 40-42 and summarized in Table 8.

TABLE 8
Effects of Alpha-Enolase Specific siRNA on the
Sensitivity of MDA-435 Tumor Cells to Drugs

| | Control | | Eno-1 | Eno-1 |
|---|---|---|---|---|
| Drug | (EC₅₀) | Mock (EC₅₀) | ST (EC₅₀) | (EC₅₀) |
| Taxol μM | 0.0011 | 0.000445 | 0.000149 | 0.000334 |
| | R2 = 0.9336 | 2.5 × IS | 7.3 × IS | 3.3 × IS |
| | | R2 = 0.9803 | R2 = 0.7789 | R2 = 0.884 |
| Vincristin μM | 0.000246 | 0.000667 | 0.000324 | 0.000375 |
| | R2 = 0.9868 | 2.7 × IR | 1.3 × IR | 1.5 × IR |
| | | R2 = 0.9776 | R2 = 0.9776 | R2 = 0.9374 |
| Docetaxel μM | 0.000645 | 0.000226 | 0.000124 | 0.000182 |
| | R2 = 0.9191 | 3.0 × IS | 5.22 × IS | 3.5 × IS |
| | | R2 = 0.990 | R2 = 0.8703 | R2 = 0.92 |
| Vinblastin μM | 0.000709 | 0.00104 | 0.000917 | 0.000872 |
| | R2 = 0.9737 | 1.5 × IS | 1.3 × IR | 1.2 × IR |
| | | R2 = 0.9876 | R2 = 0.5007 | R2 = 0.8541 |

As discussed earlier, α-enolase silencing was also effective in prostate cancer cells (FIG. 46). Using androgen-independent prostate cancer PC-3 cells, a combined effect of α-enolase silencing with taxol treatment enhanced sensitivity by 10-fold (FIG. 46). Similarly, α-enolase siRNA treatment also combined with docetaxel and vinblastine to augment PC-3 cell killing (FIG. 46, Table 9).

TABLE 9
Effects of Alpha-Enolase Specific siRNA on the
Sensitivity of PC-3 Tumor Cells to Drugs

| Chemotherapeutic Drug | Control (EC₅₀) | α-Enolase (EC₅₀) |
|---|---|---|
| Taxol nM | 0.199 | 0.0206 |
| | R2 = 0.9497 | 9.7 × IS |
| | | R2 = 0.9499 |
| Vincristin uM | 3.40 | 1.69 |
| | R2 = 0.8782 | 2.0 × IS |
| | | R2 = 0.9497 |
| Docetaxel nM | 0.348 | 0.0547 |
| | R2 = 0.9122 | 6.1 × IS |
| | | R2 = 0.9373 |
| Vinblastin nM | 0.05289 | 0.005527 |
| | R2 = 0.8790 | 9.6 × IS |
| | | R2 = 0.9333 |

Overall, silencing of α-enolase promotes the cytotoxicity of microtubule targeting drugs in human breast, ovarian, lung and colon carcinomas. The enhanced cytotoxicity observed in α-enolase depleted cells was observed in multiple tumor cell lines of diverse origins including breast, ovarian, lung, prostate and colon (for a summary see Table 10), arguing for a universal, enolase-mediated cell survival mechanism in response to microtubule targeting agents.

TABLE 10
Effects of siRNA α-enolase Transfection on the Growth of Cancer Cells

| Cell line | Doxorubicin | Taxol | Vincristin | Docetaxel |
|---|---|---|---|---|
| MCF-7 | 1.5 × IS | 10 × IS | 12 × IS | 3.5 × IS |
| MDA-435 | NC | 3.5 × IS | NC | 3.5 × IS |
| SKOV3 | NC | 2.1 × IS | 1.4 × IS | — |
| OVCAR3 | 2 × IS | 2.3 × IS | NC | — |
| CaOV3 | NC | 26 × IS | 2.5 × IS | — |
| A549 | NC | 3 × IS | 4 × IS | 20 × IS |
| H460 | NC | 2.5 × IS | 1.5 × IS | 2.5 × IS |
| SW-480 | NC | 8.4 × IS | 207 × IS | 25 × IS |
| SW-620 | NC | 7.2 × IS | 6.5 × IS | 70 × IS |
| PC-3 | NC | 10 × IS | 2 × IS | 6 × IS |

IS: “increased sensitivity” to the particular drug.
NC: indicates that no change occurred.

### Example 7

### Effects of α-Enolase Overexpression on Cell Adhesion in MCF7 Cells

To determine the effects of α-enolase on metastasis, cell adhesion studies were performed on MCF7 and H460 cell lines. MCF7 cells were transfected with the pCMV vector (Stratagene, Inc., Cedar Creek, Tex.) containing a cDNA encoding the full-length human α-enolase gene (pCMV-ENO1) obtained from McGill University, Montreal, QC, CA. Transfected cells were grown and maintained detailed in Example 1. The pCMV-ENO1 vector provided increased enolase expression as determined by Western blotting using a monoclonal antibody specific to α-enolase. Briefly, mock and α-enolase siRNA vectors were transfected into MCF7 cells and allowed to grow. Proteins were resolved on SDS-PAGE gels and transferred to nitrocellulose membranes. Membranes were then probed with α-enolase-specific monoclonal antibody, followed by a secondary antibody specific for the primary antibody.

Cell adhesion assays were performed using an MTT-based cell assay, as described in FIGS. 8C and 8D. 25 ml of MTT dye (5 mg/ml) were added into each well containing MCF7/pCMV-ENO1 cells and MCF7 mock transfectants. The plate was further incubated at 37° C. for 4 hrs. The dye was solubilized with 10% Triton X-100, 0.01 N HCl and further incubated at 37° C. in the dark for 30 min. Cell adhesion was monitored by inspecting the cells for yellow-reduced tetrazolium using a Wallac multiwell plate reader (PerkinElmer, Inc., Boston, Mass.) (FIGS. 13A and 13B). The averages of triplicate wells were plotted using the Prism software (GraphPad Software, Inc., San Diego, Calif.). The attachment of cells to the surface of the well was counted and a proportion of cells that attached to the surface during the experimental period was determined.

The results of cell-cell adhesion assays are shown in FIGS. 21A and 21B. The results shown in FIG. 21B demonstrate that increased α-enolase expression allowed the H460 cells to become unattached to other cells within the medium. The control cells transfected with a mock vector showed unaltered cell-cell adhesion, forming a confluent monolayer on the surface of the dish (FIG. 21A). Similar results were also found for MCF-7 cells and A549 cells overexpressing α-enolase (FIGS. 22A-22B and FIGS. 23A-23B, respectively).

As shown in FIG. 24, cell adhesion to laminin-coated multi-well plates was tested for A549 cell overexpressing α-enolase. These results were further confirmed by experiments showing that α-enolase silencing enhanced cell adhesion of A549 cells to collagen-coated plates (FIG. 25). The effects of α-enolase overexpression and silencing on cell adhesion in MCF-7 and A549 cells are summarized in FIGS. 26A and 26B.

### Example 8

### Effects of α-Enolase Silencing on Angiogenesis

HUVEC cells were transiently transfected with the pCMV-XL6 plasmid (OriGene Technologies, Rockville, Md.) containing the cDNA coding for the full length human α-enolase-1. Transfections were performed using Lipofectamine 2000 (Invitrogen, Inc., Carlsbad, Calif.) according to manufacturer's protocols.

Transfected cells were then subjected to an angiogenesis assay to determine the effect of α-enolase on capillary tube formation. Briefly, Matrigel-coated 24-well culture plates (BD Biosciences, Rockville, Md.) were thawed at 4° C. overnight. The Matrigel was allowed to solidify for 1 hr at 37° C. α-enolase-1-directed siRNA or control siRNA silenced HUVEC cells were subsequently seeded onto the Matrigel-coated wells at a density of 40,000 cells per well in the absence of endothelial cell growth factors. Taxol was added to selected wells at 2 nM final concentration. The cells were allowed to differentiate overnight at 37° C., and were photographed after 5-6 and 16 hours using Q-Capture software under an Olympus fluorescence microscope.

HUVEC cells transfected with control siRNA were able to form capillary tubes on the Matrigel, indicating that the cells maintained the ability to stimulate vascularization (FIGS. 32A, 45A, and 45C). However, HUVEC cells transfected with α-enolase-targeted siRNA were inhibited from forming capillary tubes on the Matrigel (FIGS. 32B, 45B, and 45D). Thus, α-enolase silencing inhibits angiogenesis, thereby preventing cells from exhibiting one of the characteristics of tumor progression.

### Example 9

### Effect of α-Enolase Silencing on Cell Invasion and Migration

Transwell filter chamber assays were performed using a Chemicon QCM 96-well invasion kit according to the manufacturer's instructions (Chemicon International, Inc., Temecula, Calif.). Eno-1 and control siRNA transfected cells were harvested 2 days post-transfection, resuspended in media without serum and seeded in 96-well Matrigel-coated transwell filter plates at 50,000 cells per well. Medium with 10% serum was used as a chemo-attractant in a feeder tray. The inserts were placed into the feeder tray, and the cells were subsequently incubated for 16 hours at 37° C. in an incubator containing a 5% CO₂ atmosphere. Cells that invaded through the filter to the bottom tray were quantitated using the CyQUANT-GR dye according to the manufacturer's instructions (Molecular Probes, Eugene, Oreg.).

These assays test the ability of a cell to move across membranes, which is a hallmark of metastatic potential. The results of transwell filter assays on A549 cells with decreased α-enolase expression are shown in FIGS. 27A and 27B. To confirm the results shown in FIGS. 27A and 27B, the highly metastatic cell line MDA-MB-435 was transfected with a vector expressing the α-enolase siRNA and subjected to the transwell filter assay (FIGS. 28 and 29, respectively).

### Example 10

### α-Enolase Targeted Therapy Against Non-Hematological Cancer Cells

In order to determine whether targeting α-enolase is useful in treating a preexisting chemotherapeutic drug-resistant cancerous condition, non-Hematological tumor cells are administered to MHC-matched mice, and tumors are allowed to form. Next, the mice are administered taxol (or another chemotherapeutic drug) at a dosage predicted to kill most, but not all of the tumor cells in the mice. Those mice that are identified as having developed chemotherapeutic drug-resistant tumor cells are administered a composition comprising taxol and a targeting agent that specifically binds to murine α-enolase messenger RNA.

The mice that receive the composition show a better prognosis (i.e., smaller tumor or fewer tumor cells) as compared to mice that receive only the targeting agent or only the vincristin. A determination of decreased tumor size or cancer cell number is made by sacrificing the mice and excising the tumor. The size of the tumor in mice treated with the α-enolase targeting agent and chemotherapy is measured and compared to measurements obtained from mice treated with chemotherapy alone. In addition, tumors are trypsinized in DMEM medium supplemented with 10% fetal bovine serum until cells are in free suspension. Cells are then transferred to six well plates for counting. Cell counts are compared. All experiments are performed in triplicate.

In further studies, the efficacy of a α-enolase-targeted therapeutic in treating an MDR mammary adenocarcinoma cells (MCF/AR) is assessed. Briefly, male thymic nude mice 5-7 weeks old, weighing 18 g-22 g are used for the MCF-7/ADR xenografts. Mice receive a subcutaneous (s.c.) injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, α-enolase siRNA alone (3 μg daily for 16 days), or both taxol and α-enolase siRNA (3 μg daily for 16 days for each treatment). Control siRNA sequences are utilized that do not represent binding sequences to murine α-enolase (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the α-enolase siRNA and chemotherapeutic drugs are compared to mice treated with control siRNA and chemotherapeutic drugs. Mice treated with the α-enolase siRNA have smaller tumors by weight than mice treated with control siRNA. In addition, total cell number in tumors isolated from mice treated with α-enolase siRNA are lower than mice treated with control siRNA.

### Example 11

### α-Enolase Targeted Therapy Against Non-Hematological Cancer Cells

In order to determine whether targeting α-enolase is useful in treating a preexisting cancerous condition, non-Hematological tumor cells are administered to MHC-matched mice, and tumors are allowed to form. Tumors are allowed to grow to a sufficient size for appropriate analysis of the effects of α-enolase treatment on tumor sensitivity to chemotherapeutic drugs. Mice are then treated with an α-enolase formulation designed to decrease the level of expression of α-enolase. The cancer cells show an increase in sensitivity to chemotherapeutic treatment regimes. As a result, the mice that receive the composition show a better prognosis (i.e., smaller tumor or fewer tumor cells) as compared to mice that receive only the targeting agent or only the vincristin. A determination of decreased tumor size or cancer cell number is made by sacrificing the mice and excising the tumor. The size of the tumor in mice treated with the α-enolase targeting agent and chemotherapy is measured and compared to measurements obtained from mice treated with chemotherapy alone. In addition, tumors are trypsinized in DMEM medium supplemented with 10% fetal bovine serum until cells are in free suspension. Cells are then transferred to six well plates for counting. Cell counts are compared. All experiments are performed in triplicate.

In further studies, the efficacy of a α-enolase-targeted therapeutic in treating an mammary adenocarcinoma cells (MCF-7) is assessed. Briefly, male thymic nude mice 5-7 weeks old, weighing 18 g-22 g are used for the MCF-7/ADR xenografts. Mice receive a subcutaneous (s.c.) injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, α-enolase siRNA alone (3 μg daily for 16 days), or both taxol and α-enolase siRNA (3 μg daily for 16 days for each treatment). Control siRNA sequences are utilized that do not represent binding sequences to murine α-enolase (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the α-enolase siRNA and chemotherapeutic drugs are compared to mice treated with control siRNA and chemotherapeutic drugs. Mice treated with the α-enolase siRNA have smaller tumors by weight than mice treated with control siRNA. In addition, total cell number in tumors isolated from mice treated with α-enolase siRNA are lower than mice treated with control siRNA.

### Example 12

### α-Enolase Liposome Formulation for Targeted Therapy Against Non-Hematological Cancer Cells

In order to determine whether α-enolase liposome formulations are useful in treating a preexisting cancerous condition, non-Hematological tumor cells are administered to MHC-matched mice, and tumors are allowed to form. Tumors are allowed to grow to a sufficient size for appropriate analysis of the effects of α-enolase treatment on tumor sensitivity to chemotherapeutic drugs. Mice are then treated with an α-enolase liposome formulation designed to decrease the level of expression of α-enolase. The cancer cells show an increase in sensitivity to chemotherapeutic treatment regimes. As a result, the mice that receive the composition show a better prognosis (i.e., smaller tumor or fewer tumor cells) as compared to mice that receive only the targeting agent or only the vincristin. A determination of decreased tumor size or cancer cell number is made by sacrificing the mice and excising the tumor. The size of the tumor in mice treated with the α-enolase targeting agent and chemotherapy is measured and compared to measurements obtained from mice treated with chemotherapy alone. In addition, tumors are trypsinized in DMEM medium supplemented with 10% fetal bovine serum until cells are in free suspension. Cells are then transferred to six well plates for counting. Cell counts are compared. All experiments are performed in triplicate.

In further studies, the efficacy of a α-enolase-targeted therapeutic in treating an mammary adenocarcinoma cells (MCF-7) is assessed. Briefly, male thymic nude mice 5-7 weeks old, weighing 18 g-22 g are used for the MCF-7/ADR xenografts. Mice receive a subcutaneous (s.c.) injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, α-enolase siRNA/liposome formulation alone (3 μg daily for 16 days), or both taxol and α-enolase siRNA/liposome formulation (3 μg daily for 16 days for each treatment).

Liposome formulations are produced as described previously (Shi and Pardridge (2000) Proc. Natl. Acad. Sci. USA. 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer (pH=8.0) and sonicated for 10 min. α-enolase siRNA is added to the lipids. The liposome/siRNA dispersion is evaporated to a final concentration of 200 mM at a volume of 100 μl. The dispersion is frozen in ethanol/dry ice for 4-5 min. The dispersion is then thawed at 40° C. for 1-2 min, and this freeze-thaw cycle is repeated 10 times. The liposome dispersion is diluted to a lipid concentration of 40 mM, is followed by extrusion 10 times each through two stacks each of 400-nm, 200-nm, 100-nm, and 50-nm pore size polycarbonate membranes, by using a hand held extruder (Avestin, Ottawa). The mean vesicle diameters are determined by quasielastic light scattering by using a Microtrac Ultrafine Particle Analyzer (Leeds-Northrup, St. Petersburg, Fla.).

Control siRNA sequences are utilized that do not represent binding sequences to murine α-enolase (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the α-enolase siRNA and chemotherapeutic drugs are compared to mice treated with control siRNA and chemotherapeutic drugs. Mice treated with the α-enolase siRNA have smaller tumors by weight than mice treated with control siRNA. In addition, total cell number in tumors isolated from mice treated with α-enolase siRNA are lower than mice treated with control siRNA.

### Example 13

### α-Enolase Formulations for Targeted Therapy Against Non-Hematological Cancer Cells

In order to determine whether α-enolase formulations are useful in treating a preexisting cancerous condition, non-Hematological tumor cells are administered to MHC-matched mice, and tumors are allowed to form. Tumors are allowed to grow to a sufficient size for appropriate analysis of the effects of α-enolase treatment on tumor sensitivity to chemotherapeutic drugs. Mice are then treated with an α-enolase formulation designed to decrease the level of expression of α-enolase. The present formulations allow for improved targeting of the α-enolase siRNA treatment to the cancer cells, thereby increasing the efficacy of the siRNA treatment.

Treatment with the α-enolase siRNA formulations improves chemotherapeutic treatment. The cancer cells show an increase in sensitivity to chemotherapeutic treatment regimes. As a result, the mice that receive the composition show a better prognosis (i.e., smaller tumor or fewer tumor cells) as compared to mice that receive only the targeting agent or only the vincristin. A determination of decreased tumor size or cancer cell number is made by sacrificing the mice and excising the tumor. The size of the tumor in mice treated with the α-enolase targeting agent and chemotherapy is measured and compared to measurements obtained from mice treated with chemotherapy alone. In addition, tumors are trypsinized in DMEM medium supplemented with 10% fetal bovine serum until cells are in free suspension. Cells are then transferred to six well plates for counting. Cell counts are compared. All experiments are performed in triplicate.

In further studies, the efficacy of a α-enolase-targeted therapeutic in treating an mammary adenocarcinoma cells (MCF-7) is assessed. Briefly, male thymic nude mice 5-7 weeks old, weighing 18 g-22 g are used for the MCF-7/ADR xenografts. Mice receive a subcutaneous (s.c.) injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, α-enolase siRNA formulation alone (3 μg daily for 16 days), or both taxol and α-enolase siRNA formulation (3 μg daily for 16 days for each treatment).

α-enolase formulations are produced as described previously (Shi and Pardridge (2000) Proc. Natl. Acad. Sci. USA. 97(13): 7567-7572). Briefly, POPC (19.2 μmol), DDAB (0.2 μmol), phosphatidylethanolamine (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer (pH=8.0) and sonicated for 10 min. α-enolase siRNA is added to the lipids. The liposome/siRNA dispersion is evaporated to a final concentration of 200 mM at a volume of 100 μl. The dispersion is frozen in ethanol/dry ice for 4-5 min. The dispersion is then thawed at 40° C. for 1-2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering by using a Microtrac Ultrafine Particle Analyzer (Leeds-Northrup, St. Petersburg, Fla.).

Plasminogen proteins are attached to the surface of the liposome/siRNA formulations using methods similar to those described previously (see U.S. Pat. No. 4,762,915). Briefly, liposomal/siRNA suspensions (1.5 μM) are mixed with EDCI (4 mg) in 1.5 ml of 10 mM NaPO₄, 0.15M NaCl, pH 5.0. The reaction is carried out at room temperature for one hr. The liposome/siRNA mixture (1.5 ml) is mixed with 75 μl of plasminogen (10 mg/ml) and 75 μl of 1 M NaCl, and the coupling-reaction mixture is adjusted to pH 8.0. Each reaction is carried out overnight at 4° C. Unreacted protein is separated from liposome-conjugated protein by metrizamide density gradient centrifugation. Control coupling reactions are performed by substituting buffer for liposomal/siRNA complexes. The amount of protein bound to the liposomes is determined by the Lowry protein assay. The concentration of liposomal lipid was determined from I¹²⁵ radioactivity levels, based on a known amount of PE-I¹²⁵ included in the liposome preparations. Based on the measured protein and lipid concentrations, the protein to lipid coupling ratios, expressed in μg protein/μg mole, lipid are determined. This preparation is then administered to the animals.

Control siRNA sequences are utilized that do not represent binding sequences to murine α-enolase (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the α-enolase siRNA and chemotherapeutic drugs are compared to mice treated with control siRNA and chemotherapeutic drugs. Mice treated with the α-enolase siRNA have smaller tumors by weight than mice treated with control siRNA. In addition, total cell number in tumors isolated from mice treated with α-enolase siRNA are lower than mice treated with control siRNA.

### Example 14

### α-Enolase Immunoliposome Formulation for Targeted Therapy Against Non-Hematological Cancer Cells

In order to determine whether α-enolase immunoliposome formulations are useful in treating a preexisting cancerous condition, non-Hematological tumor cells are administered to MHC-matched mice, and tumors are allowed to form. Tumors are allowed to grow to a sufficient size for appropriate analysis of the effects of α-enolase treatment on tumor sensitivity to chemotherapeutic drugs. Mice are then treated with an α-enolase immunoliposome formulation designed to decrease the level of expression of α-enolase. The immunoliposome formulation allows for increased efficacy of treatment by targeting cancer cells. The cancer cells show an increase in sensitivity to chemotherapeutic treatment regimes. As a result, the mice that receive the composition show a better prognosis (i.e., smaller tumor or fewer tumor cells) as compared to mice that receive only the targeting agent or only the vincristin. A determination of decreased tumor size or cancer cell number is made by sacrificing the mice and excising the tumor. The size of the tumor in mice treated with the α-enolase targeting agent and chemotherapy is measured and compared to measurements obtained from mice treated with chemotherapy alone. In addition, tumors are trypsinized in DMEM medium supplemented with 10% fetal bovine serum until cells are in free suspension. Cells are then transferred to six well plates for counting. Cell counts are compared. All experiments are performed in triplicate.

In further studies, the efficacy of a α-enolase-targeted therapeutic in treating an mammary adenocarcinoma cells (MCF-7) is assessed. Briefly, male thymic nude mice 5-7 weeks old, weighing 18 g-22 g are used for the MCF-7/ADR xenografts. Mice receive a subcutaneous (s.c.) injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, α-enolase siRNA/immunoliposome formulation alone (3 μg daily for 16 days), or both taxol and α-enolase siRNA/immunoliposome formulation (3 μg daily for 16 days for each treatment).

Immunoliposome formulations are produced as described previously (Shi and Pardridge (2000) Proc. Natl. Acad. Sci. USA. 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer (pH=8.0) and sonicated for 10 min. α-enolase siRNA is added to the lipids. The liposome/siRNA dispersion is evaporated to a final concentration of 200 mM at a volume of 100 μl. The dispersion is frozen in ethanol/dry ice for 4-5 min. The dispersion is then thawed at 40° C. for 1-2 min, and this freeze-thaw cycle is repeated 10 times. The liposome dispersion is diluted to a lipid concentration of 40 mM, is followed by extrusion 10 times each through two stacks each of 400-nm, 200-nm, 100-nm, and 50-nm pore size polycarbonate membranes, by using a hand held extruder (Avestin, Ottawa). The mean vesicle diameters are determined by quasielastic light scattering by using a Microtrac Ultrafine Particle Analyzer (Leeds-Northrup, St. Petersburg, Fla.).

An anti-nucleophosmin mAb is harvested from serum-free nucleophosmin hybridoma-conditioned media. The anti-nucleophosmin mAb, as well as the isotype control, mouse IgG2a, are purified by protein G Sepharose affinity chromatography. The anti-nucleophosmin mAb or mouse IgG2a (1.5 mg, 10 nmol) is thiolated by using a 40:1 molar excess of 2-iminothiolane (Traut's reagent), as described previously (Huwyler et al. (1996) Proc. Natl. Acad. Sci. USA. 93:14164-14169). Thiolated mAB is conjugated to pegylated liposomes using standard procedures described previously (Huwyler et al. (1996) Proc. Natl. Acad. Sci. USA. 93:14164-14169). This preparation is then administered to the animals.

Control siRNA sequences are utilized that do not represent binding sequences to murine α-enolase (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the α-enolase siRNA and chemotherapeutic drugs are compared to mice treated with control siRNA and chemotherapeutic drugs. Mice treated with the α-enolase siRNA have smaller tumors by weight than mice treated with control siRNA. In addition, total cell number in tumors isolated from mice treated with α-enolase siRNA are lower than mice treated with control siRNA.

### Example 15

### α-Enolase Expression in Select Tumors

Although initially identified as a differentially expressed protein in drug sensitive and resistant tumor cells, it was of interest to determine if alpha-enolase expression was also altered between normal and tumor human cells. To address this question, approximately 330 tissue samples from well defined normal and malignant tumor samples were obtained and the levels of alpha-enolase were compared.

Tissue samples were obtained from two commercial tissue providers ( ). Ovarian samples were obtained from 62 tumor patients and 77 normal individuals. In addition, tissue samples were obtained from 11 lung cancer patients and 15 normal lung samples. Breast tissue samples from 61 normal samples and 79 tumors were analyzed. All tissues were processed identically as described below.

Total RNA Quality Controls

Total RNA was quantified with the Nanodrop® ND-1000 spectrophotometer and the A260/A280 ratio calculated. Only total RNA samples (tumor model systems or patient samples) with a A260/A280 ratio between 1.9-2.3 in 10 mM Tris pH 7.5 were included in the study. The integrity of the ribosomal RNAs was visualized on standard 1% agarose electrophoresis in TBE buffer (Tris 9 mM; 9 mM Borate Acid; 0.2 mM EDTA) containing 0.04% EtBr. Only RNA samples without RNA degradation and with good rRNA 28S/18S ratio were used for first strand cDNA labeling reaction.

First Strand cDNA Labeling Reaction

Microarray reference model was used in that study. Moreover, a dye swap reaction is also performed for each patient sample on the same day to account for the potential differential incorporation of the Cye-dCTP dyes used in the first strand cDNA labeling reaction. Although, a highly recommended step by all careful users of Microarray technology, the reverse labeling is one of the steps that is skipped by many. The labeling reaction was done with 10 μg total RNA from tumor samples and the corresponding normal pool of normal or individual normal samples. In brief, total RNA was incubated with 2 ng RNA of Arabidopsis thaliana (positive control), 3 μg Oligo (dT)₁₂₋₁₈ primer (Invitrogen, USA), 1 μg PdN₆ random primer (Amersham, USA) for 10 min at 65° C. and on ice for 2 min. The sample was then diluted in the labeling reaction buffer (5× First strand buffer [50 mM Tris-HCl pH 8.3; 75 mM KCl; 3 mM MgCl₂]; 20 mM DTT; 0.5 mM dATP; 0.5 mM dTTP; 0.5 mM dGTP; 0.05 mM dCTP; 26 μM Cy5-dCTP or 52 μM Cy3-dCTP) (NEN Life Science, Perkin Elmer, USA) and 400 U SuperScript III RNAse H—RT (Invitrogen, USA). Samples are incubated for 5 min at 25° C. followed by a reaction step of 90 min at 42° C. 400 U of SuperScript II RNAse H⁻ RT is added and the reaction was continued for another 90 min.

Digestion of the labeled cDNA with 5 U RNAse H (NEB, USA) and 40 U RNAse A (Amersham, USA) was done at 37° C. for 30 min. The labeled probe was purified with the QIAquick PCR purification kit (QIAgen, USA) protocol with some modifications. In brief, the reaction volume was completed to 50 μL with the addition of DEPC H₂O and 2.7 μL 2 M NaOAc pH 5.2. Samples were diluted with 200 μL PB buffer and purified on spin columns. Samples were spun for 20 sec at 10 000 g, followed by 3 washes of 500 μL PE buffer (20 sec; 10 000 g) and eluted twice with 25 μL DEPC H₂O (50 μL total) (1 min; 10 000 g). The frequency of incorporation and amount of cDNA labeled produced were evaluated for both labeled dCTPs by spectrophotometer (Nanodrop® ND-1000, USA) at 260 nm, 550 nm and 650 nm. The labeled material was dried by speed vacuum (Savant SC110A, USA) and resuspended in 3.75 μL H₂O for both Cy5-dCTP and Cy3-dCTP labeled samples.

Hybridization Conditions

BioChip slide was pre-washed before the pre-hybridization step as followed: 20 min at 42° C. in preheated 2×SSC (300 mM NaCl; 30 mM Sodium citrate)/0.2% SDS under agitation, 5 min at room temperature in 0.2×SSC (30 mM NaCl, 3 mM Sodium citrate) under agitation and 5 min at room temperature in DEPC H₂O with agitation. The slide was spin dry at 1000 g for 5 min and pre-hybridized in Dig Easy Hyb Buffer (Roche, USA) containing 0.02% Bovine Serum Albumin (Roche, USA) at 42° C. in humid chamber for 3 hrs then washed 2 times in DEPC H₂O, 1 time in Isopropanol (Sigma, USA) and spin dry at 1000 g for 5 min. Baker tRNA (15 μg; Roche, USA) and 1 μg Cot-1 DNA (Roche, USA) were added to the labeled probe and the mixture was incubated 5 min at 95° C., put on ice for 1 min and diluted with 14 μL Dig Easy Hyb buffer (Roche, USA). The sample was spun for 2 min at 100 g and final samples were incubated at 42° C. for at least 5 min.

In an effort to screen more than one patient sample at a time, several methods were tested to simultaneously test multiple samples on a single microscope slide. Following considerable testing and optimization, 3 super-grids were chosen to spot on a single slide, each super-grid separated by a Jet-Set Quick Dry TOP Coat 101 line (L'OREAL, Paris #FX268). Each probe was added to its respective super-grid and covered by a preheated (42° C.) coverslip (Mandel, USA). The slide was incubated at 42° C. in humid chamber for at least 15 hrs. The coverslips were removed by dipping in 1×SSC (150 mM NaCl; 15 mM Sodium citrate) containing 0.2% SDS preheated at 50° C., then washed 3 times 5 min in 1×SSC (150 mM NaCl; 15 mM Sodium citrate) containing 0.2% SDS solution preheated at 50° C. with agitation, 3 times in 0.1×SSC (15 mM NaCl; 1.5 mM Sodium citrate)/0.2% SDS solution preheated at 37° C. with agitation and 1 time in 0.1×SSC (15 mM NaCl; 1.5 mM Sodium citrate) with agitation for 5 min. The slide was dipped several times in DEPC H₂O and spun-dry at 1000 g for 5 min.

Statistical Analysis

For data analysis, slides were scanned with the ScanArray™ Lite MicroArray Scanner (Packard BioSciences, Perkin Elmer) and results analyzed with QuantArray® Microarray Analysis software version 3.0 (Packard BioSciences, Perkin Elmer), using the adaptive method. The QuantArray® results were analyzed as follows: a) analysis of the results was done with the spot background subtracted values for Cy5 and Cy3 channels; b) Spots with lower signal ratio to noise lower than 1.5 were discarded; c) Intense signals are adjusted to a minimum of 100 and spots with signal value lower than 100 in both channel were discarded. Normalization of ratio values were achieved with spiked positive control (Arabidopsis thaliana) to have a ratio equal to one for that control on each slide. Slides with negative and/or positive controls that did not fall in the latter control values were discarded. Average of the ratios for each target was done between the direct and the reciprocal labeling reaction. Statistical analysis was done with the ArrayStat 1.0 (Imaging Research Inc.). A log transformation of the ratio data was followed by a Student T test for two independent conditions using a proportional model without offsets at a p<0.05 threshold. Significant increase (ratio Cy5/Cy3 higher than 2.0) or decrease (ratio Cy5/Cy3 lower than 0.5) were considered to be significant if the p-value is lower than 0.05.

Supervised Hierarchical Analysis

Class prediction analyses were performed using the BRB ArrayTools developed by Dr. Richard Simon (NIH/NCI) and Amy Peng. In brief, class prediction analyses were done on the results obtained for each patient in the study. Patients were divided into two classes following their malignancy: normal class and tumor class. Class determination was done based on the clinical data associated to each patient. BRB ArrayTools software is offering 6 different classification algorithms: Compound covariate predictor, Diagonal linear discriminant analysis, Nearest neighbor predictor (1-NN and 3-NN), Nearest centroid predictor and Support vector machine predictor. These analyses allowed the development of a multi-gene classifier to predict the class for a new sample and estimate the mis-classification rates. Cross-validation of the class prediction classifiers were done by the leave one-out study and permutation tests (n=2000) were conducted to address significance of the cross-validation test error rate.

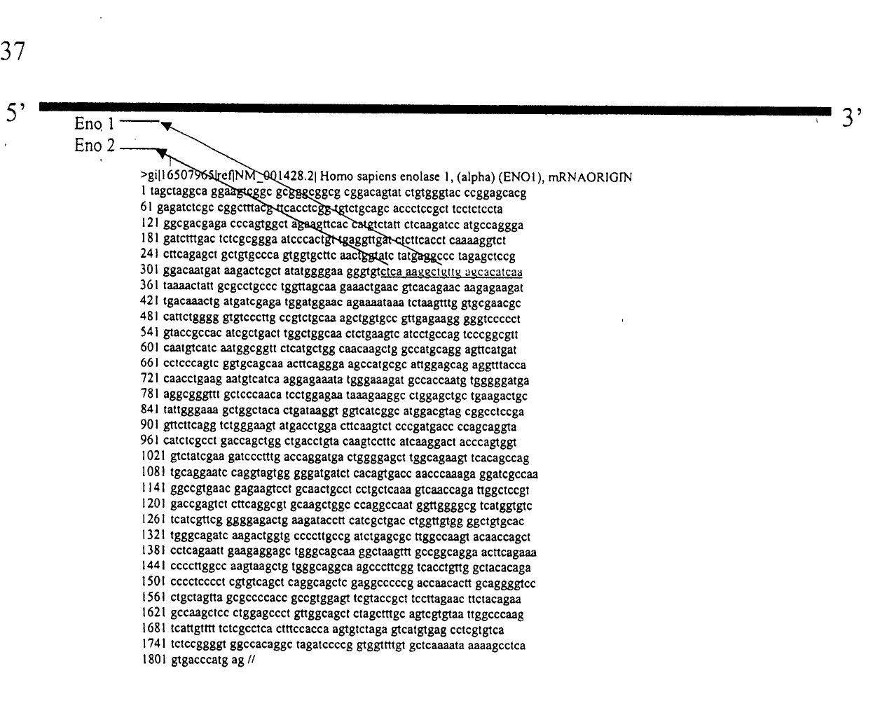

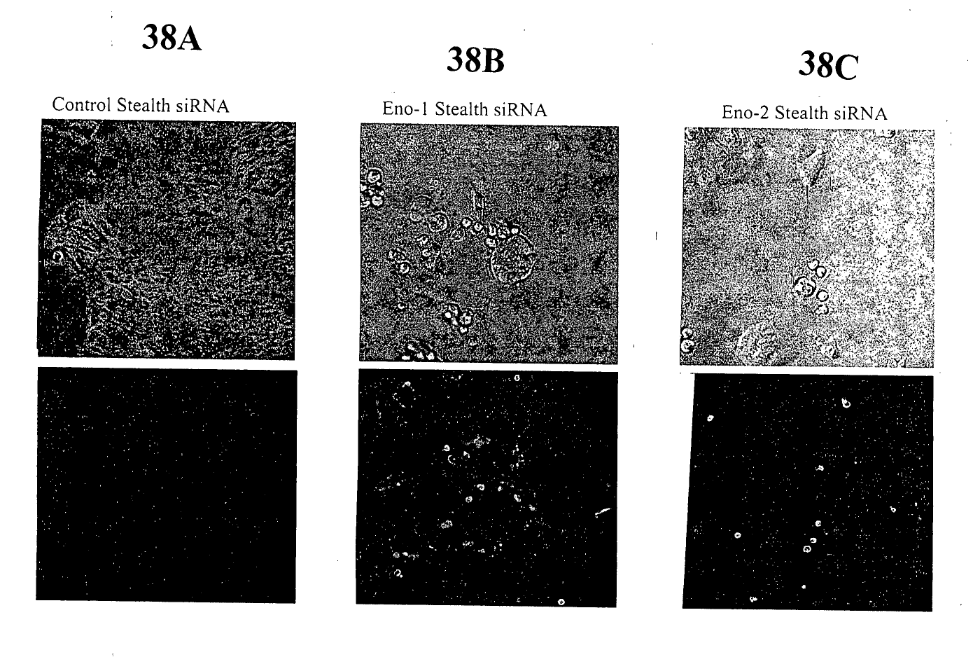

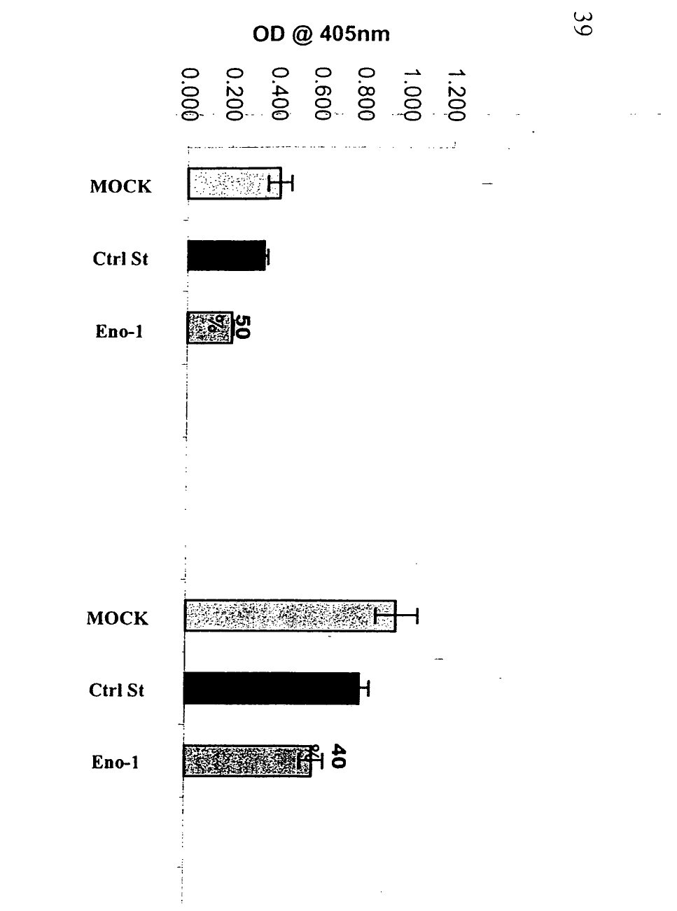

Alpha-enolase mRNA levels were significantly higher in all ovarian tumors obtained from cancer patients at various stages of malignancies (62 tumors) by comparison to normal ovarian tissues from patients (77 patients) (FIG. 34, (p<0.001)). The mean of normal to normal pool ratio was 0.3 while that of malignant ovarian tumors to normal pool ratio was 1.6 (FIG. 34). Similarly, alpha-enolase expression was measured to be dramatically higher in tumors from patients with lung cancer (NSCLC) (11 patients) by comparison to normal lung samples (15 patients) (FIG. 35, (p<0.001)). The mean expression of alpha-enolase in normal to normal-pool had a ratio of 1.0 while that of malignant lung to normal-pool had a ratio of 5.0 (FIG. 35). Analysis of alpha enolase mRNA expression in tumors from breast cancer patients versus normal breast tissue also showed higher mRNA expression in tumors (61 patients) with a mean ratio of 2 to 1.2 in normal (FIG. 36, (p<0.001)).

### Example 16

### Effects of α-Enolase Silencing on Cell Survival of Normal, Non-Malignant Cells

Detroit skin, Hfl fetal lung, and MRC-5 fetal lung fibroblast cell lines were treated with α-enolase-targeted siRNA as described in Example 5. Cell survival was determined using the MTT cytotoxicity assays described in Example 6.

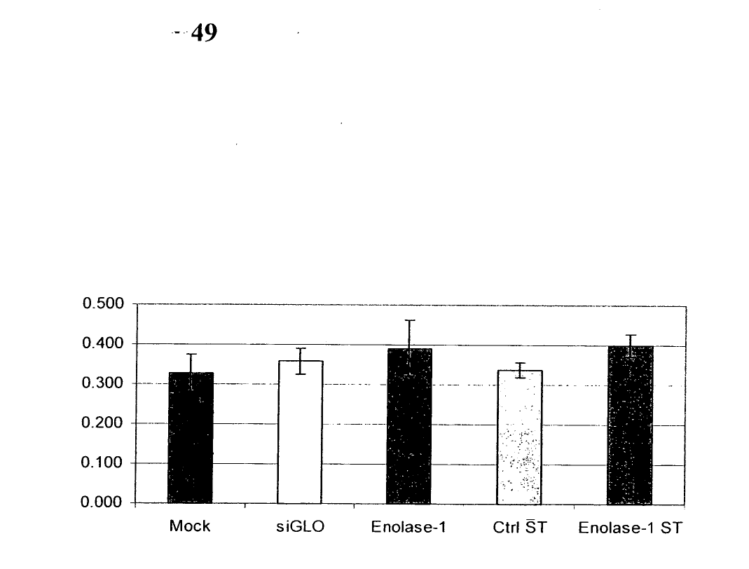

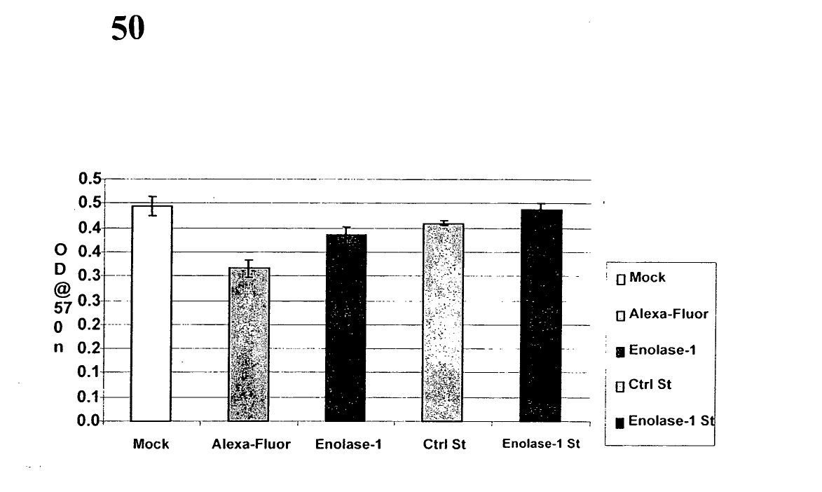

Silencing with α-enolase in Detroit fibroblast cells led to a significant down-regulation of α-enolase after three days of treatment (FIG. 47). After six days, the levels of expression of α-enolase were increased to near normal levels (FIG. 47). Silencing of α-enolase in normal fibroblast cells had no effect on survival, relative to mock or control siRNA treated normal fibroblasts nor did such down regulation lead to increased sensitivity of normal fibroblast to chemotherapeutic drugs (FIGS. 48-50).

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method of diagnosing a neoplastic cell, comprising:
a) detecting a level of α-enolase expressed in a test cell sample, the test cell sample potentially containing a neoplastic cell selected from the group consisting of breast adenocarcinoma, small cell lung carcinoma, large cell lung carcinoma, lymphoblastic leukemia cells, chronic myelogeneous leukemia cells, acute promyelocytic leukemia cells, ovarian carcinoma, ovarian adenocarcinoma, and prostate adenocarcinoma;
b) detecting a level of α-enolase expressed in a normal cell sample of the same tissue type as the test cell sample; and
c) comparing the level of expressed α-enolase in the test cell sample to the level of expressed α-enolase in the normal cell sample,
wherein the test cell sample is neoplastic if the level of α-enolase expressed therein is greater than the level of α-enolase expressed in the normal cell sample.

2. The method of claim 1, wherein detecting the levels of expressed α-enolase in the test cell sample comprises isolating a cytoplasmic sample from the test cell sample.

3. The method of claim 1, wherein detecting the level of expressed α-enolase in the test cell sample comprises contacting the test cell sample with an α-enolase targeting agent selected from the group consisting of a ligand, a synthetic small molecule, a nucleic acid, a peptidomimetic compound, an inhibitor, a peptide, a protein, and an antibody.

4. The method of claim 3, wherein the α-enolase targeting agent comprises an anti-α-enolase antibody or an α-enolase binding fragment thereof.

5. The method of claim 4, wherein the level of antibody bound to α-enolase is detected by immunofluorescence, radiolabel, or chemiluminescence.

6. The method of claim 1, wherein detecting the level of expressed α-enolase in the neoplastic cell comprises hybridizing a nucleic acid probe to a complementary α-enolase mRNA expressed in the test cell sample.

7. The method of claim 6, wherein the nucleic acid probe is selected from the group consisting of RNA, DNA, RNA-DNA hybrids, and siRNA.

8. The method of claim 3, wherein the level of α-enolase targeting agent is detected by labeling the targeting agent with a label selected from the group consisting of fluorophores, chemical dyes, radiolabels, chemiluminescent compounds, colorimetric enzymatic reactions, chemiluminescent enzymatic reactions, magnetic compounds, and paramagnetic compounds.

9. The method of claim 1, wherein the test cell sample is isolated from a mammal.

10. The method of claim 9, wherein the test cell sample is isolated from a human.

11. The method of claim 6, wherein the neoplastic cell is a breast adenocarcinoma.

12. The method of claim 6, wherein the neoplastic cell is a lung carcinoma.

13. The method of claim 6, wherein the neoplastic cell is a lymphoblastic leukemia cell.

14. The method of claim 1, wherein the test cell sample is isolated from a tissue selected from the group consisting of breast, skin, lymphatic, prostate, bone, blood, brain, liver, thymus, kidney, lung, and ovary.

15. The method of claim 1, wherein the detection steps comprise detecting the level of a cell surface-expressed α-enolase in the test cell sample and in the normal cell sample.

16. The method of claim 15, wherein the cell surface-expressed α-enolase is detected with an α-enolase targeting agent.

17. The method of claim 16, wherein the cell-surface-expressed α-enolase is detected with an anti-α-enolase antibody or an α-enolase binding fragment thereof.

18. The method of claim 16, wherein the α-enolase targeting agent comprises plasminogen.

19. The method of claim 16, wherein the α-enolase targeting agent comprises an inhibitor of α-enolase selected from the group consisting of phosphonoacetohydroxamate, (3-hydroxy-2-nitropropyl)phosphonate, (nitroethyl)phosphonate, and (phosphonoethyl)nitrolate.

20. The method of claim 16, wherein the α-enolase targeting agent is detected using a label selected from the group consisting of fluorophores, chemical dyes, radiolabels, chemiluminescent compounds, colorimetric enzymatic reactions, chemiluminescent enzymatic reactions, magnetic compounds, and paramagnetic compounds.

21. A method of diagnosing chemotherapeutic drug resistance in a neoplastic cell, comprising:
a) detecting a level of α-enolase expressed in a potentially chemotherapeutic drug-resistant neoplastic cell sample selected from the group consisting of breast adenocarcinoma, small cell lung carcinoma, large cell lung carcinoma, lymphoblastic leukemia cells, chronic myelogeneous leukemia cells, acute promyelocytic leukemia cells, ovarian carcinoma, ovarian adenocarcinoma, and prostate adenocarcinoma;
b) detecting a level of α-enolase expressed in a non-chemotherapeutic drug-resistant neoplastic cell of the same tissue type as the potentially drug-resistant neoplastic cell sample; and
c) comparing the level of expressed α-enolase in the potentially drug-resistant neoplastic cell sample to the level of expressed α-enolase in the non-drug-resistant neoplastic cell of the same tissue type,
wherein the potentially drug-resistant neoplastic cell sample is chemotherapeutic drug-resistant if the level of α-enolase expressed therein is greater than the level of α-enolase expressed in the non-chemotherapeutic-drug-resistant neoplastic cell.

22. A method of diagnosing or detecting metastatic potential and/or angiogenic phenotype of a neoplastic cell sample, comprising:
a) detecting a level of expressed α-enolase in the potentially metastatic and/or angiogenic neoplastic cell sample;
b) detecting a level of expressed α-enolase in a nonmetastatic, nonangiogenic neoplastic cell sample of the same tissue type; and
c) comparing the level of expressed α-enolase detected in the potentially metastatic and/or angiogenic neoplastic cell sample to the level of expressed α-enolase in the nonmetastatic, nonangiogenic neoplastic cell sample,
wherein metastatic potential and/or an angiogenic phenotype is indicated if the level of expressed α-enolase in the potentially metastatic and/or angiogenic neoplastic cell sample is greater than the level of expressed α-enolase in the nonmetastatic, nonangiogenic neoplastic cell sample.

23. A method of treating a neoplasm in a patient, comprising:
a) administering an effective amount of an α-enolase targeting agent to the patient, the targeting agent binding to α-enolase expressed by the neoplasm; and
b) administering to the patient an effective amount of a chemotherapeutic drug,
wherein the α-enolase targeting agent, when bound to the neoplasm, increases the sensitivity of the neoplasm to the chemotherapeutic drug.

24. A kit for detecting a level of expression of α-enolase in a neoplastic cell sample, comprising:
a) a first probe specific for α-enolase;
b) a second probe for the detection of chemotherapeutic drug resistance, the second probe being specific for a marker selected from the group consisting of vimentin, HSC70, and nucleophosmin; and
c) a detection means for identifying probe binding to a target.

25. A pharmaceutical formulation for treating a neoplasm, comprising:
a) an α-enolase-specific targeting component;
b) a chemotherapeutic drug; and
c) a pharmaceutically acceptable carrier.

26. The pharmaceutical formulation of claim 25, wherein the α-enolase-specific targeting component is selected from the group consisting of ligands, nucleic acids, synthetic small molecules, peptidomimetic compounds, inhibitors, peptides, proteins, and antibodies.

27. The pharmaceutical formulation of claim 26, wherein the α-enolase-specific targeting component is a nucleic acid.

28. The pharmaceutical formulation of claim 27, wherein the nucleic acid is selected from the group consisting of RNA, DNA, RNA-DNA hybrids, and siRNA.

29. The pharmaceutical formulation of claim 28, wherein the siRNA comprises 18 contiguous nucleotides of SEQ ID No: 2.

30. The pharmaceutical formulation of claim 28, wherein the siRNA comprises 25 contiguous nucleotides selected from the group consisting of SEQ ID No: 4 and SEQ ID No: 6.

31. The pharmaceutical formulation of claim 26, wherein the α-enolase targeting component comprises an antibody or α-enolase-binding fragment thereof.

32. The pharmaceutical formulation of claim 26, wherein the α-enolase-specific targeting component comprises an inhibitor of α-enolase selected from the group consisting of phosphonoacetohydroxamate, (3-hydroxy-2-nitropropyl)phosphonate, (nitroethyl)phosphonate, and (phosphonoethyl)nitrolate.

33. The pharmaceutical formulation of claim 25, wherein the α-enolase-specific targeting component comprises a liposome.

34. The pharmaceutical formulation of claim 33, wherein the liposome comprises a neoplastic cell-targeting component on its surface.

35. The pharmaceutical formulation of claim 34, wherein the neoplastic cell-targeting component is an antibody, or α-enolase-binding fragment thereof, that binds to a neoplastic cell marker selected from the group consisting of multidrug resistance protein 1, BRCP, p53, vimentin, α-enolase, nucleophosmin, and HSC70.

36. The pharmaceutical formulation of claim 34, wherein the neoplastic cell-targeting component comprises plasminogen.

37. The pharmaceutical formulation of claim 25, wherein the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine, and combinations thereof.


SEQUENCE LISTING (20 sequences)

SEQ ID NO: 1 - RNA, 19 nt, Artificial Sequence
  Description of Artificial Sequence Synthetic oligonucleotide
ggcuguugagcacaucaau

SEQ ID NO: 2 - RNA, 19 nt, Artificial Sequence
  Description of Artificial Sequence Synthetic oligonucleotide
auugaugugcucaacagcc

SEQ ID NO: 3 - RNA, 24 nt, Artificial Sequence
  Description of Artificial Sequence Synthetic oligonucleotide
cucaaaggcuguugagacaucaau

SEQ ID NO: 4 - RNA, 25 nt, Artificial Sequence
  Description of Artificial Sequence Synthetic oligonucleotide
auugaugugcucaacagccuuugac

SEQ ID NO: 5 - RNA, 25 nt, Artificial Sequence
  Description of Artificial Sequence Synthetic oligonucleotide
ccaguggugcuucaacugguaucua

SEQ ID NO: 6 - RNA, 25 nt, Artificial Sequence
  Description of Artificial Sequence Synthetic oligonucleotide
uucuuuggucugcauucacauuugu

SEQ ID NO: 7 - RNA, 25 nt, Artificial Sequence
  Description of Artificial Sequence Synthetic oligonucleotide
ccaggguuccuaaucggauuugcua

SEQ ID NO: 8 - RNA, 25 nt, Artificial Sequence
  Description of Artificial Sequence Synthetic oligonucleotide
acaaaugugaaugcagaccaaagaa

SEQ ID NO: 9 - RNA, 25 nt, Artificial Sequence
  Description of Artificial Sequence Synthetic oligonucleotide
uucuuuggucugcauucacauuugu

SEQ ID NO: 10 - DNA, 1812 bp, Homo sapiens
tagctaggcaggaagtcggcgcgggcggcgcggacagtatctgtgggtacccggagcacggagatctcgccggctttacgttcacctcggtgtctgcagcaccctccgcttcctctcctaggcgacgagacccagtggctagaagttcaccatgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagcccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagctttgcagtcgtgtaattggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgag

SEQ ID NO: 11 - DNA, 21 bp, Artificial Sequence
  Description of Combined DNA/RNA Molecule Synthetic oligonucleotide
  Description of Artificial Sequence Synthetic oligonucleotide
ggcuguugagcacaucaautt

SEQ ID NO: 12 - DNA, 21 bp, Artificial Sequence
  Description of Combined DNA/RNA Molecule Synthetic oligonucleotide
  Description of Artificial Sequence Synthetic oligonucleotide
auugaugugcucaacagcctt

SEQ ID NO: 13 - protein, 9 aa, Artificial Sequence
  Description of Artificial Sequence Synthetic peptide
SGKYDLDFK

SEQ ID NO: 14 - protein, 10 aa, Artificial Sequence
  Description of Artificial Sequence Synthetic peptide
IGAEVYHNLK

SEQ ID NO: 15 - protein, 12 aa, Artificial Sequence
  Description of Artificial Sequence Synthetic peptide
YISPDQLADLYK

SEQ ID NO: 16 - protein, 15 aa, Artificial Sequence
  Description of Artificial Sequence Synthetic peptide
FGANAILGVSLAVCK

SEQ ID NO: 17 - protein, 14 aa, Artificial Sequence
  Description of Artificial Sequence Synthetic peptide
VVIGMDVAASEFFR

SEQ ID NO: 18 - protein, 17 aa, Artificial Sequence
  Description of Artificial Sequence Synthetic peptide
LAMQEFMILPVGAANFR

SEQ ID NO: 19 - protein, 20 aa, Artificial Sequence
  Description of Artificial Sequence Synthetic peptide
AGYTDKVVIGMDVAASEFFR

SEQ ID NO: 20 - protein, 22 aa, Artificial Sequence
  Description of Artificial Sequence Synthetic peptide
YGKDATNVGDEGGFAPNILENK